(19) United States
(12) Patent Application Publication
(10) Pub. No.: US 2024/0139104 A1
(43) Pub. Date: May 2, 2024

(54) MICELLE RELEASING THERMOSENSITIVE HYDROGELS AS A THERAPEUTIC DELIVERY SYSTEM

(71) Applicants: Susan Napier THOMAS, Atlanta, GA (US); GEORGIA TECH RESEARCH CORPORATION, Atlanta, GA (US)

(72) Inventors: Susan Napier THOMAS, Atlanta, GA (US); Jihoon KIM, Atlanta, GA (US)

(21) Appl. No.: 18/280,397

(22) PCT Filed: Mar. 4, 2022
(86) PCT No.: PCT/US2022/018923

§ 371 Date: Sep. 5, 2023

Related U.S. Application Data
(60) Provisional application No. 63/157,350, filed on Mar. 5, 2021.
(60) Provisional application No. 63/300,733, filed on Jan. 19, 2022.

(51) Int. Cl. A61K 9/06; A61K 9/19; A61K 45/06; A61K 47/10; A61K 47/60; A61P 35/00

(52) U.S. Cl. CPC A61K 9/06; A61K 9/19; A61K 45/06; A61K 47/10; A61K 47/60; A61P 35/00

(57) ABSTRACT

Disclosed herein are thermosensitive hydrogels including at least one thermosensitive polymer and at least one polypeptide. The hydrogels may further include one or more therapeutic and/diagnostic agents and can be used to selectively and controllably deliver such agents to specified tissues and organ systems.

### CROSS-REFERENCE TO RELATED APPLICATION

This application claims the benefit of U.S. Provisional Application 63/300,733, filed Jan. 19, 2022, and 63/157,350, filed Mar. 5, 2021, the contents of each are hereby incorporated in their entireties.

### STATEMENT OF GOVERNMENT SUPPORT

This invention was made with government support under Grant No. W81XWH-16-1-0518 awarded by the Department of Defense and Grant No. R01CA207619 awarded by the National Institutes of Health. The government has certain rights in the invention.

### FIELD OF THE INVENTION

The invention is directed to thermosensitive hydrogels, methods of making thermosensitive hydrogels, and methods of using thermosensitive hydrogels. In certain embodiments, the thermosensitive hydrogels exist in the sol state at room temperature, and gel at higher temperatures. In certain embodiments, the thermosensitive hydrogels may be used as drug delivery systems for a variety of different therapeutic and/or diagnostic agents. In some embodiments, the thermosensitive hydrogel may be implanted in a subject while in the sol state, and subsequent to injection transitions to the gel state. In some embodiments, the thermosensitive hydrogel may be used for the controlled release of therapeutic and/or diagnostic agents in a subject over time. In certain embodiments, the thermosensitive hydrogels degrade in vivo while releasing micelles, which may include one or more therapeutic and/or diagnostic agent. In some embodiments, the thermosensitive hydrogels may be used to selectively deliver therapeutic and/or diagnostic agents to the tumor and lymphatic system, or to any other system which selectively absorbs the micelles.

### BACKGROUND

Various drug carriers have been developed to deliver therapeutic agents. In general, a drug carrier is loaded with drugs, through mechanical or chemical means, and is then injected or implanted into the subject for controlled release of the drugs. Depending on applications, the drug carrier may be loaded with one or more therapeutic and diagnostic agents. Hydrogels have been used as drug carriers due in part to their high water content and biocompatibility.

Cancer immunotherapy operates by encouraging the intrinsic immune system to fight the cancer. As highlighted in Nobel Prize in Medicine in 2018, several immune checkpoint blockade monoclonal antibodies (mAB) has been recently approved by Food and Drug Administration (FDA), such as Ipilimumab (Yervoy®), Pembrolizumab (Keytrude®), Nivolumab (Opdivo®), Atezolizumab (Tecentriq®), Avelumab (Bevancio®), and Durvalumab (Imfinzi®). These immune checkpoint blockade monoclonal antibodies (mAB) antagonistically bind to the cytotoxic T-lymphocyte antigen-4 (CTLA-4), programmed death-1 (PD-1), or programmed death-1 ligands (PD-L1) that prevent the antitumoral immune response in the process of DC to T cell prime or in the action phase of T cells on tumors. In addition, several other immune checkpoint blockade antibodies also have been under development, such as lymphocyte-activation gene 3 (LAG-3), T cell immunoglobulin and mucin-domain containing-3 (TIM-3), T cell immunoglobulin and ITIM domain (TIGIT), and so on.

Despite the efficient therapeutic effects of immune checkpoint blockade antibodies and their successful clinical translations over traditional radiation and chemotherapy, the therapeutic efficacy, response rates, and safety of these antibodies could be improved. Indeed, 40-85% of patients do not respond to immune checkpoint blockade antibody therapy, and there have been continuous reports regarding severe or life-threatening immune-related adverse events associated with them. As a result, there have been continuous efforts to address these issues not only by developing more safe and efficient drug delivery systems (DDSs), but also by combining immune checkpoint blockade antibodies with other therapy including radiation, chemotherapeutic drugs, chimeric antigen receptor T cells (CAR-T), cytokines, antigens, adjuvants, and so on.

Nitric oxide (NO) is an endogenous gas molecule that is synthesized via enzymatic reactions with Nitric Oxide Synthases (NOSs) and L-arginine substrates in our body. This nitric oxide has a variety of biological and pathophysiological functions in vivo such as cardiovascular homeostasis, neurotransmission, cell proliferation, apoptosis, angiogenesis, and immune response. These various functions of NO can be modulated by the concentrations and release duration. For example, low NO concentration with long NO releasing duration functions as vasodilation, anti-thrombosis, angiogenesis, wound healing and cardiovascular homeostasis, whereas the opposite condition allows apoptotic activity and antibacterial activity. That is, exogenously delivered NO is considered as a promising ideal drug that not only exerts therapeutic effects, but also minimizes side effects because of its endogenous presence in vivo and rapid transformation into innocuous ions within six seconds or less after its action. In addition, there have been continuous efforts in exploiting NO in combination therapy based on its ability to inhibit intracellular drug efflux, to improve apoptosis of cancer cells, and to enhance drug accumulations in tumor via tumor vascular vasodilation. However, most NO-delivering drugs have failed due to the enormous effects of NO on various physiological and biological functions, as well as the difficulty in selectively delivering the optimized dose of drug to its in vivo target.

BRAF is an important molecule in cell growth, differentiation, and apoptosis, which is associated with EGF, KRAS, BRAF, MEK, ERK, PI3K, and mTOR pathways. Although BRAF monomer leads to low activity on its downstream MEK, it is dimerized to exert elevated MEK activity when its upstream RAS is activated. However, the mutation of BRAF-kinase monomer in ATP-binding pocket leads to consecutive unchecked activation of RAS/RAF/MEK/ERK pathway, which changes growth, differentiation, and survival of cancer cells. BRAF-mutated tumors account for ˜5.5% of all new US cancer cases, half of melanoma cases are BRAF-mutated, and in particular, most common mutation is BRA^{V600E} (˜80%). Therefore, there have been continuous development of strategy to treat BRAF-mutated tumor in clinic, such as EGFR inhibitors (Cetuximab, Panitumumab), IGF1R inhibitors (Ganitumab), KRAS inhibitors (Dacomitinib), BRAF inhibitors (Vemurafenib, Debrafenib), and MEK inhibitors (Trametinib, Binimetinib) that intend to block upstream and downstream of BRAF pathways. Vemurafenib and Debrafenib have been approved for the treatment of BRAF-mutated tumor. Although transdermal administrations of BRAF inhibitors led to the suppression of tumor growth in preclinical xenograft tumor models, the acquired resistance has been arisen as an inevitable problem of BRAF inhibitors-mediated therapy. In immune-competent preclinical models and in clinics, therapeutic effects of BRAF inhibitors are demonstrated to be mainly associated with elevated CD8⁺T cells and NK cells, and loss of MDSCs and regulatory T cells (L_{regS}). However, PD-L1expression is increased on cancer cells and NKG2D ligands that is a ligand to NKG2D to mediate cytotoxicity of NK and T cells are downregulated on tumor (Int. J. Cancer 2020, 146, 1409-1420.) as a counter part of expansion and improved tumor infiltration of CD8⁺T and NK cells, which reduce the immune-mediated therapeutic efficacy of BRAF inhibitors. Accordingly, immune checkpoint blockades (ICBs) have been highlighted to improve the therapeutic efficacy of BRAF inhibitors by invigorating the antitumor immune responses.

Surgical removal of primary and metastatic tumor tissues is primary treatment options if possible, followed by cancer immunotherapy. Since tumor draining lymph nodes (TdLNs) are the primary route in cancer metastasis, detection and surgical removal of TdLNs as well as primary tumors govern the prognosis of patients in anticancer therapy. However, it is difficult to find TdLNs among numerous lymph nodes (LNs) that are small and are distributed throughout the body. Unfortunately, X-ray computed tomography (CT), magnetic resonance imaging (MRI), and positron emission tomography (PET) requires large facilities and long acquisition time, which cannot be utilized to detect and complete surgical dissections of TdLNs during surgery in real-time. On the other hands, near infrared dyes (NIR) dyes including indocyanin green and methylene blue have been approved by Food and Drug Administration (FDA) of USA and European Medicines Agency (EMA) in image-guided surgery supported by potable intraoperative fluorescence imaging devices. However, low photostability and aqueous stability, and short blood circulations of NIR dyes have limited their versatile and wide usage in clinic.

There remains a need for improved drug delivery systems providing controllable release of therapeutics. There remains a need for improved drug delivery systems for delivering therapeutic agents to targeted tissues/cells in a subject. There remains a need for effectively delivering near infrared (NIR) imaging agents to the lymph for the early detection of cancers and other diseases. There remains a need for improved regimens of cancer immunotherapy. Especially in cancer immunotherapy, there remains a need for improved methods of combining BRAF inhibitors, NO-delivering drugs, and immune checkpoint inhibitors, amongst other agents, including a need for improved compositions containing such therapeutic agents.

In accordance with the purposes of the disclosed materials and methods, as embodied and broadly described herein, the disclosed subject matter, in one aspect, relates to compounds, compositions and methods of making and using compounds and compositions.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

The details of one or more embodiments are set forth in the descriptions below. Other features, objects, and advantages will be apparent from the description and from the claims.

### BRIEF DESCRIPTION OF THE FIGURES

FIG. 1 depicts a polypeptide backbone having multiple COOH (for example from glutamic acid and aspartic acid residues) and NH₂ groups (from lysine or arginine residues, also include asparagine, glutamine, and histidine (each of which include an NH instead of NH₂ group), wherein the thermosensitive polymer PEG-PPG-PEG is first activated with a molar excess of carboxyl donor (e.g., 4-nitrophenyl chloroformate), and then reacted with the amine groups of gelatin to give a hydrogel having a mixture of singly linked and crosslinked linked thermosensitive polymer to polypeptide.

FIG. 2 depicts ¹H NMR of F127-g-gelatin in D₂O.

FIG. 3 depicts quantitative ¹H NMR analysis of F127-g-gelatin. (A) ¹H NMR of physical mixture of gelatin and F127 at a different weight ratio. F127 and gelatin were marked with red and blue colors, respectively. Arg and Lys peaks of gelatin are marked as a blue box I. Glu and Hyp peaks of gelatin are marked as a blue box II. Val, Leu, and Ile peaks of gelatin are marked as a blue box III. Methyl groups of polypropylene oxide in F127 are marked as a red box. (B-D) Integration ratio of red box to each blue box were plotted according to the Gelatin to F127 weight ratio. The composition of F127-g-gelatin was calculated and averaged by using each standard curve (B-D).

FIG. 4 depicts the temperature sensitivity of the sol/gel transition of a F127-g-gelatin hydrogel.

FIG. 5 depicts the sol-gel transition properties of (A) F127, (B) gelatin, and (C) admixture of gelatin and F127.

FIG. 6 depicts the XRD of lyophilized (A) gelatin, (B) F127, (C) mixture of gelatin and F127 (Gelatin:F127=35.1 wt. %:64.9 wt. %), and (D) F127-g-gelatin at the concentration of 4 wt. %.

FIG. 7 depicts powder DSC thermograms of lyophilized (A) gelatin (T_{c}=N.D., and Tₘ=N.D.), (B) F127 (T_{c}=27.2° C., and Tₘ=53.3° C.), (C) mixture of gelatin and F127 (gelatin:F127=35.1 wt. %:64.9 wt. %) (T_{c}=24.7° C., and Tₘ=48.6° C.), and (D) F127-g-Gelatin (T_{c}=25.5° C., and Tₘ=48.7° C.) at the concentration of 4 wt. %. T_{c} and Tₘ represents the crystalline temperature and melting temperature, respectively. N.D. means “not determined.”

FIG. 8 depicts solution DSC thermograms of solutions containing (A) deionized water, (B) gelatin, (C) F127, (D) mixture of gelatin and F127 (Gelatin:F127=35.1 wt. %:64.9 wt. %), and (E) F127-g-gelatin at the concentration of 4 wt. %.

FIG. 9 depicts a pyrene-assisted ratiometric CMC (critical micelle concentration) determination of (A) F127 at room temperature (RT), (B) mixture of gelatin and F127 (gelatin:F127=35.1 wt. %:64.9 wt. %) at RT, (C) F127-g-gelatin at RT, (D) F127 at 37° C., (E) mixture of gelatin and F127 (gelatin:F127=35.1 wt. %:64.9 wt. %) at 37° C., and (F) F127-g-gelatin at 37° C. (excitation at 336 nm and emission at 373 nm and 383 nm). The intersection of two distinctive linear lines represents CMC₁. Gelatin does not show any distinctive intersections for CMC₁. Data are presented as mean±SD (n=3-5).

FIG. 10 depicts images of F127-g-gelatin hydrogel. (A) Photo images of F127-g-gelatin thermosensitive hydrogel at 4.5 wt. % and 37° C. (B) SEM images of lyophilized 4.5 wt. % F127-g-gelatin.

FIG. 11 depicts the concentration-dependent storage (G′) and loss (G″) modulus of F127-g-gelatin at 37° C. Data are presented as mean±SD (n=3−5).

FIG. 12 depicts (A) storage (G′), (B) loss (G″), and (C) loss tangent (G″/G′) of F127-g-gelatin of F127-g-Gelatin at different concentrations at 37° C. Data are presented as mean±SD (n=3−5).

FIG. 13 depicts the preparation of Alexa Fluor™ 647 labeled aCTLA-4. (A) Mild stirring of aCTLA-4 in PBS and Alexa Fluor™ 647 NHS Ester (AF647-NHS) in DMSO allowed the synthesis of aCTLA-4-AF647. (B) Pure aCTLA-4-AF647 was yielded with CL-6B Sepharose® column and Amicon® Ultra centrifugal filter (Millipore, MWCO 10 kDa).

FIG. 14. (A, B) Cumulative release of (A) GSNO (0.45 mg mL⁻¹) (n=4) and (B) Alexa Fluor™ 647 labeled aCTLA-4 (0.542 mg mL⁻¹) (n=3) from 4.5 wt % F127-g-Gelatin (300 μL) in 300 μL PBS with or without MMP-9 (n=4). (C, D) In vitro stability test of F127-g-Gelatin (4.5 wt %, 300 μL) containing (C) GSNO (0.45 mg mL⁻¹) (n=4) or (D) Alexa Fluor™ 647 labeled aCTLA-4 (0.542 mg mL⁻¹) (n=3) in 300 μL PBS with or without MMP-9. Data are presented as mean±SD. ****p<0.0001, ***p<0.001, **p<0.01, and *p<0.05 with two-way ANOVA using Tukey post-hoc statistical hypothesis.

FIG. 15 depicts DLS and zeta potential of aCTLA-4, supernatants released from bare F127-g-gelatin hydrogel (F127-g-gelatin), and supernatants released from F127-g-gelatin hydrogel containing aCTLA-4 (aCTLA-4/F127-g-Gelatin) (n=12), which were obtained after hydrogels were totally disrupted (F127-g-gelatin and aCTLA-4 concentrations equivalent to 0.9 wt. % and 0.542 mg mL⁻¹, respectively). The left and right insets represent the average size and zeta potentials of materials, respectively. Data are presented as mean±SD. ****p<0.0001, ***p<0.001, **p<0.01, and *p<0.05 with one-way ANOVA using Tukey post-hoc statistical hypothesis.

FIG. 16 depicts pyrene-assisted ratiometric CMC determination of F127-g-gelatin containing aCTLA-4 and aCTLA-AF647 (excitation at 336 nm and emission at 373 nm and 383 nm). The intersections of two distinctive linear lines represent CMC₁ and CMC₂. CMC₁ and CMC₂ of (A) F127-g-gelatin containing aCTLA at 37° C., and (B) F127-g-gelatin containing aCTLA-AF647 at 37° C. (C, D) Quantitative and statistical analysis of CMC₁ and CMC₂ obtained from (A,B), respectively. Data are presented as mean±SD (n=3).

FIG. 17 depicts the preparation of TRITC labeled aCTLA-4 and FITC labeled F127-g-gelatin. (A) Mild stirring of aCTLA-4 in PBS and TRITC in DMSO at room temperature allowed the synthesis of TRITC labeled aCTLA-4. Pure TRITC labeled aCTLA-4 was yielded with CL-6B Sepharose® column and Amicon® Ultra centrifugal filter (Millipore, MWCO 10 kDa). (B) Mild stirring of F127-g-gelatin in PBS and FITC in DMSO at room temperature allowed the synthesis of FITC labeled F127-g-gelatin. Pure FITC labeled F127-g-gelatin was yielded with CL-6B Sepharose® column and Amicon® Ultra centrifugal filter (Millipore, MWCO 10 kDa).

FIG. 18 depicts a FRET analysis to investigate the interactions between aCTLA-4-TRITC and F127-g-gelatin-FITC at FITC excitation and TRITC emission (n=4). Data are presented as mean±SD.

FIG. 19 depicts the size distribution of F127 micelles with and without aCTLA-4 (n=12). Final concentrations of F127 and aCTLA-4 concentrations are 0.9 wt. % and 0.542 mg mL⁻¹, respectively, which are equivalent to the concentrations of F127-g-gelatin and aCTLA-4 in FIG. 41.

FIG. 20 depicts a competitive assay to verify the activity of aCTLA-4 released from F127-g-gelatin hydrogel (n=4). B16F10 intrinsically expresses CTLA-4. After pre-treating B16F10 with free aCTLA-4 or supernatant released from F127-g-gelatin hydrogels (aCTLA-4 concentrations equivalent to 0.88 mg mL⁻¹), the cells were stained with CTLA-4-BV605. The decrease of fluorescence represents the binding of the pretreated aCTLA-4. Data are presented as mean±SD. ****p<0.0001, ***p<0.001, **p<0.01, and *p<0.05 with one-way ANOVA using Tukey post-hoc statistical hypothesis.

FIG. 21 depicts a cell viability test of NIH3T3 and B16F10-OVA in the treatment with F127-g-gelatin. Each 10⁴ (A) NIH3T3 or (B) B16F10-OVA treated with F127-g-gelatin in 96 well cell culture plate was incubated for 2 days, followed by 1 h incubation with alamarBlue™ cell viability reagent. Fluorescence (560 nm excitation, 590 nm emission) was recorded with Synergy H4 microplate reader. ****p<0.0001, ***p<0.001, **p<0.01, and *p<0.05 with one-way ANOVA using Tukey post-hoc statistical hypothesis by comparing it with 0 mg mL⁻¹. Data are presented as mean±SD (n=8 for A, and n=4-5 for B).

FIG. 22 depicts the relative body weight changes after one-time subcutaneous administration of bare F127-g-gelatin hydrogel on tumor-free mice. Data are presented as mean±SEM (n=5). ****p<0.0001, ***p<0.001, **p<0.01, and *p<0.05 with one-way ANOVA using Tukey post-hoc statistical hypothesis by comparing it with day 0.

FIG. 23 depicts ALT/AST activities of plasma taken from mice 2 d after administrations of 4.5 wt. % F127-g-gelatin hydrogel (n=5). ****p<0.0001, ***p<0.001, **p<0.01, and *p<0.05 with two-tailed Student t-test.

FIG. 24 depicts representative time-resolved IVIS® images of mice treated with free Alexa Fluor™ 647 labeled aCTLA-4 (aCTLA-4-AF647) or F127-g-gelatin containing aCTLA-4-AF647. The inversed values of aCTLA-4 not diffused from the injection sites represent the amounts of aCTLA-4 released from the injection sites. Data are presented as mean±SEM (n=4).

FIG. 25 depicts (A) In vivo stability of 4.5 wt. % F127-g-gelatin hydrogel by quantifying the remained at the injection sites (n=4). (B) In vivo quantification of aCTLA-4-AF647 remained at the injection sites with or without 4.5 wt. % F127-g-gelatin hydrogel by utilizing IVIS® (n=4). Data are presented as mean±SEM. ****p<0.0001, ***p<0.001, **p<0.01, and *p<0.05 with two-way ANOVA using Tukey post-hoc statistical hypothesis.

FIG. 26 depicts the biodistributions of free aCTLA-4-AF647, aCTLA-4-AF647 with 0.45 wt. % F127-g-gelatin micelles (aCTLA-4 micelle), and aCTLA-4-AF647 with 4.5 wt. % F127-g-gelatin hydrogel (aCTLA-4 dose equivalent to 162 μg mouse⁻¹) (n=4) in (A) 1º tumor, (B) 1º dLN, (C) 2º tumor, (D) 2º dLN, (E) Blood, (F) Spleen, (G) Liver, (H) Kidney, and (I) Lung. Data are presented as mean±SEM. ****p<0.0001, ***p<0.001, **p<0.01, and *p<0.05 with one-way ANOVA using Tukey post-hoc statistical hypothesis.

FIG. 27 depicts in vivo therapeutic effects of aCTLA-4 with F127-g-gelatin hydrogel in intradermal (i.d.) injection to the tissue ipsilateral (i.l.) to the tumor. (A) Outline of tumor model and treatment schedule. 1º and 2º tumor was inoculated with B16F10-OVA 100,000 cells in 30 μL saline on day 0 and day 4, respectively. 300 μg aCTLA-4 in 30 μL saline was administered on day 7. (B) 1º tumor size during treatment (n=5). (C) 2º tumor size during treatment (n=5). (D) Kaplan-Meier survival curves during treatment (n=5). Data are presented as mean±SEM. ****p<0.0001, ***p<0.001, **p<0.01, and *p<0.05 with two-way ANOVA using Tukey post-hoc statistical hypothesis.

FIG. 28 depicts in vivo systemic therapeutic effects of combinational use of GSNO and aCTLA-4. (A) Outline of tumor model and treatment schedule. 1º and 2º tumor was inoculated with B16F10-OVA10⁵ cells in 30 μL saline on day 0 and day 4, respectively. GSNO (480 μg kg⁻¹) in 30 μL saline was intratumorally treated on day 7, and aCTLA-4 (100 μg mouse⁻¹) in 30 μL saline was intraperitoneally administered on day 8, 11, and 14. Blood was harvested from facial vein on day 13 for blood immune cell profiles. (B) 1º tumor size during treatment (n=5-6). (C) 2º tumor size during treatment (n=5-6). (D) Relative body weight during treatment (n=5-6). (E) Kaplan-Meier survival curves during treatment (n=5-6). Data are presented as mean±SEM. ****p<0.0001, ***p<0.001, **p<0.01, and *p<0.05 with two-way ANOVA using Tukey post-hoc statistical hypothesis.

FIG. 29 depicts populations of (A) CD45⁺ (n=4-5), (B) CD4⁺T (CD45⁺CD3⁺CD4⁺, n=4-5), (C) LAG-3⁺CD4⁺T (n=4-5), (D) PD-1⁺CD4⁺T (n=4-5), (E) CD8⁺T (CD45⁺CD3⁺CD8⁺, n=4-5), (F) CD25⁺CD8⁺T (n=4-5), (G) LAG-3⁺CD8⁺T (n=4-5), (H) PD-1⁺CD8⁺T (n=4-5), (I) Tetramer CD8⁺T (n=4-5), (J) CD45⁺CD3⁻NK1.1⁺(NK) (n=4-5), (K) CD45⁺CD3⁺NK1.1⁺(NKT) (n=4-5), and (L) CD45⁺CD3⁺CD4⁺CD25⁺Foxp3⁺(T_{reg}) (n=4-5). Data are presented as mean±SEM (n=4-5). Data are presented as mean±SEM. ****p<0.0001, ***p<0.001, **p<0.01, and *p<0.05 with one-way ANOVA using Tukey post-hoc statistical hypothesis.

FIG. 30 depicts in vivo systemic therapeutic effects of GSNO and aCTLA-4 loaded F127-g-gelatin hydrogel. (A) Outline of tumor model and treatment schedule. 1º and 2º tumor were inoculated with B16F10-0VA 10⁵ cells in 30 μL saline on day 0 and day 4, respectively. GSNO (570 μg kg⁻¹) and aCTLA-4 (100 μg mouse⁻¹) in 30 μL 4.5 wt. % F127-g-gelatin hydrogel were intratumorally treated on day 7. Blood was harvested from facial vein on day 9 for ALT/AST assay. (B) Relative body weight during treatment (n=5). (C) ALT/AST activity of blood on day 9 (n=5). (D) Kaplan-Meier survival curves during treatment (n=5). (E) 1º tumor size during treatment (n=5). (F) 2º tumor size during treatment (n=5). ****p<0.0001, ***p<0.001, **p<0.01, and *p<0.05 with two-way ANOVA using Tukey post-hoc statistical hypothesis for B,E,F. ****p<0.0001, ***p<0.001, **p<0.01, and *p<0.05 with one-way ANOVA using Tukey post-hoc statistical hypothesis for C.

FIG. 31 depicts the conditions of HPLC to quantify Vem released from hydrogels. (A) time dependent gradient changes of acetonitrile and deionized water in HPLC systems. (B) Overall HPLC peaks of each sample including PBS, acetonitrile, F127-g-Gelatin, MMP, and Vem. Vem was detected on 12.3 min. (C) Concentration-dependent measurement of Vem in the HPLC. (D) Concentration-dependent standard curves of Vem, which was quantified from the area of each curve in (C). Data are presented as mean±SD.

FIG. 32 depicts (A-C) In vitro residence stability test of (A) bare (n=3-4), (B) Vem (0.67 mg mL⁻¹) (n=3), and (C) TRITC labeled aPD-1 (0.672 mg mL⁻¹) (n=3-4) from 4.5 wt % F127-g-Gelatin (300 μL) in 300 μL PBS with or without MMP-9. (D, E) Cumulative release of (D) Vem (0.67 mg mL⁻¹) (n=3), and (E) TRITC labeled aPD-1 (0.672 mg mL⁻¹) (n=3-4) from 4.5 wt. % F127-g-gelatin (300 μL) into 300 μL PBS with or without MMP-9. Data are presented as mean±SD. ****p<0.0001, ***p<0.001, **p<0.01, and *p<0.05 with two-way ANOVA using Tukey post-hoc statistical hypothesis.

FIG. 33 depicts an investigation of interaction between F127-g-gelatin and Vem or F127-g-gelatin and aPD-1. (A, B) Polymer concentrations-dependent ratiometric emitted fluorescence (373 nm and 383 nm) of pyrenes at excitation wavelength of 336 nm was recorded. The intersections of two distinctive linear lines represent CMC. CMC of (A) F127-g-Gelatin containing pyrene and pyrene+Vem at RT, and (B) F127-g-gelatin containing pyrene and pyrene+Vem at 37° C. (C) Polymer concentrations-dependent TRITC fluorescence of aPD-1-TRITC (547 nm excitation, 579 nm emission). Data are presented as mean±SD (n=3).

FIG. 34 depicts biodistributions of free aPD-1-AF647, free Vem+aPD-1-AF647, aPD-1-AF647 with 4.5 wt. % F127-g-gelatin hydrogel, and Vem+aPD-1-AF647 with 4.5 wt. % F127-g-gelatin hydrogel (aPD-1 and Vem dose equivalent to 100 μg mouse⁻¹, and 10 mg kg⁻1, respectively) (n=4) in (A) tumor, (B) tumor draining lymph node (TdLN), (C) non draining lymph node (ndLN), (D) Spleen, (E) Liver, (F) Kidney, (G) Lung, and (H) Blood. Data are presented as mean±SEM. ****p<0.0001, ***p<0.001, **p<0.01, and *p<0.05 with one-way ANOVA using Tukey post-hoc statistical hypothesis.

FIG. 35 depicts in vivo therapeutic effects of Vem and aPD-1 loaded F127-g-gelatin hydrogel. (A) Outline of tumor model and treatment schedule. D4M tumor was inoculated with 5×10⁵ D4M cells in 30 μL saline on day 0. Vem (20 mg kg⁻¹) and aPD-1 (300 μg mouse⁻¹) in 30 μL 4.5 wt. % F127-g-gelatin hydrogel was intratumorally treated on day 7. Blood was harvested from facial vein on day 14 for ALT/AST assay. (B) Tumor size during treatment (n=5). (C) ALT/AST assay (n=5). (D) Relative body weight during treatment (n=5). (E) Kaplan-Meier survival curves during treatment (n=5). ****p<0.0001, ***p<0.001, **p<0.01, and *p<0.05 with two-way ANOVA using Tukey post-hoc statistical hypothesis for B,D. ****p<0.0001, ***p<0.001, **p<0.01, and *p<0.05 with one-way ANOVA using Tukey post-hoc statistical hypothesis for C.

FIG. 36 depicts populations of (A) CD4⁺T (CD45⁺CD3⁺CD4⁺) from CD45⁺ in dLN (n=4-5), (B) CD4⁺T from CD45⁺ in spleen (n=4-5), (C) T_{reg} (CD45⁺CD3⁺CD4⁺Foxp3⁺) from CD45⁺ in dLN (n=4-5), (D) T_{reg} from CD45⁺ in spleen (n=4-5), (E) T_{reg} from CD4⁺T in dLN (n=4-5), and (F) (E) T_{reg} from CD4⁺T in spleen (n=4-5). Data are presented as mean±SEM (n=4-5). ****p<0.0001, ***p<0.001, **p<0.01, and *p<0.05 with one-way ANOVA using Tukey post-hoc statistical hypothesis.

FIG. 37 depicts populations of (A) CD8⁺T (CD45⁺CD3⁺CD8⁺) from CD45⁺ in dLN (n=4-5), and (B) CD8⁺T from CD45⁺ in spleen (n=4-5). Data are presented as mean±SEM (n=4-5). ****p<0.0001, ***p<0.001, **p<0.01, and *p<0.05 with one-way ANOVA using Tukey post-hoc statistical hypothesis.

FIG. 38 depicts populations of (A) CD4⁺T from CD45⁺ in tumor (n=4-5), (B) T_{reg} from CD45⁺ in tumor (n=4-5), (C) T_{reg} from CD4⁺T in tumor (n=4-5), (D) CD8⁺T from CD45⁺ in tumor (n=4-5), (E) DC from CD45⁺ in tumor (n=4-5), (F) T_{CM} (CD45⁺CD3⁺CD8⁺CD62L⁺CD44⁺) from CD45⁺ in tumor (n=4-5), and (G) T_{EM} (CD45⁺CD3⁺CD8⁺CD62L^{-CD}44⁺) from CD45⁺ in tumor (n=4-5). Data are presented as mean±SEM (n=4-5). ****p<0.0001, ***p<0.001, **p<0.01, and *p<0.05 with one-way ANOVA using Tukey post-hoc statistical hypothesis.

FIG. 39 depicts populations of (A) DC from CD45⁺ in dLN (n=4-5), and (B) DC from CD45⁺ in spleen (n=4-5). Data are presented as mean±SEM (n=4-5). ****p<0.0001, ***p<0.001, **p<0.01, and *p<0.05 with one-way ANOVA using Tukey post-hoc statistical hypothesis.

FIG. 40 depicts populations of (A) T_{CM} from CD45⁺ in dLN (n=4-5), (B) T_{CM} from CD45⁺ in spleen (n=4-5), (C) T_{EM} from CD45⁺ in dLN (n=4-5), and (D) T_{EM} from CD45⁺ in spleen (n=4-5). Data are presented as mean±SEM (n=4-5). ****p<0.0001, ***p<0.001, **p<0.01, and *p<0.05 with one-way ANOVA using Tukey post-hoc statistical hypothesis.

FIG. 41 depicts populations of (A) Tₛₜₑₘ₋ₗᵢₖₑ (CD45⁺CD3⁺CD8⁺Tcf1⁺PD-1⁺Tim3⁻) from CD45⁺ in dLN (n=4-5), (B) Tₛₜₑₘ₋ₗᵢₖₑ from CD45⁺ in spleen (n=4-5), (C) Tₛₜₑₘ₋ₗᵢₖₑ from CD45⁺ in tumor (n=4-5), (D) T_{eff-like} (CD45⁺CD3⁺CD8⁺Tcf1⁻PD-1⁺Tim3⁺) from CD45⁺ in dLN (n=4-5), (E) T_{eff-like} from CD45⁺ in spleen (n=4-5), and (F) T_{eff-like} from CD45⁺ in tumor (n=4-5). Data are presented as mean±SEM (n=4-5). ****p<0.0001, ***p<0.001, **p<0.01, and *p<0.05 with one-way ANOVA using Tukey post-hoc statistical hypothesis.

FIG. 42 depicts the photostability of IR780 loaded in F127-g-Gelatin thermosensitive hydrogel. (A) UV-vis spectra and fluorescence spectra of IR780 loaded in F127-g-Gelatin thermosensitive hydrogel. (B) UV-vis spectra of free IR780 exposed to daylight. (C) UV-vis spectra of IR780 loaded in F127-g-Gelatin thermosensitive hydrogel. (D) Fluorescence spectra of free IR780 exposed to daylight. (E) Fluorescence spectra of IR780 loaded in F127-g-Gelatin thermosensitive hydrogel exposed to daylight. (B-E) Intensity decreased overtime. That is, each graph for 0 h, 2 h, 4 h, and 24 h is presented from top to bottom

FIG. 43 depicts IR780 loading micelles released from F127-g-Gelatin thermosensitive hydrogel. (A) Residence stability of F127-g-Gelatin hydrogel loading IR780. (B) IR780 release profiles from F127-g-Gelatin hydrogel loading IR780. (C) Correlation graph between hydrogel degradation and IR780 release. (D) MMP-9 responsive degradation of F127-g-Gelatin hydrogel loading IR780. (E) MMP-9 responsive IR780 release from F127-g-Gelatin hydrogel loading IR780. (F) TEM image of micelles in situ released from F127-g-Gelatin. (G) TEM image of micelles in situ released from F127-g-Gelatin loading IR780. Data are presented as a mean±SD with statistical analysis using two-way ANOVA supported Tukey post-hoc hypothesis; ****p<0.0001, ***p<0.001, **p<0.01, and *p<0.05.

FIG. 44 depicts surgery simulation of tumor draining lymph nodes using F127-g-Gelatin hydrogel loading IR780 in a single tumor model. Single tumor models were established by inoculating 105 B16F10-OVA cells in 30 μL saline to right dorsal of C57B1/6 mice on day 0. (A-C) IVIS® images of sacrificed mouse 24 h after 15 μL (A) Saline, (B) Free IR780 (0.016 mg mL⁻¹), and (C) F127-g-Gelatin thermosensitive hydrogel (4.5 wt. %) loading IR780 (0.016 mg mL⁻¹) were administered intratumorally on day 7. White arrows and white dot circles represent injection sites and draining lymph nodes, respectively. Excised axial draining lymph nodes, brachial draining lymph nodes, axial non-draining lymph nodes, and brachial draining lymph nodes were placed in order in the below each mouse image.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

As used herein, “control” is an alternative subject or sample used in an experiment for comparison purposes and included to minimize or distinguish the effect of variables other than an independent variable. A “control” can be positive or negative.

As used herein, “therapeutic” generally refers to treating, healing, and/or ameliorating a disease, disorder, condition, or side effect, or to decreasing in the rate of advancement of a disease, disorder, condition, or side effect. The term also includes within its scope enhancing normal physiological function, palliative treatment, and partial remediation of a disease, disorder, condition, side effect, or symptom thereof.

The terms “treating” and “treatment” as used herein refer generally to obtaining a desired pharmacological and/or physiological effect. The effect may be prophylactic in terms of preventing or partially preventing a disease, symptom, or condition thereof.

As used interchangeably herein, “subject,” “individual,” or “patient,” refers to a vertebrate, preferably a mammal, more preferably a human. Mammals include, but are not limited to, murine, simians, humans, farm animals, sport animals, and pets. The term “pet” includes a dog, cat, guinea pig, mouse, rat, rabbit, ferret, and the like. The term farm animal includes a horse, sheep, goat, chicken, pig, cow, donkey, llama, alpaca, turkey, and the like.

As used herein, “administration” refers to the injection of active agent on the subject. Exemplary methods of administration include: intravenously (i.v.), intraperitoneally (i.p.), intratumorally (i.t.), or subcutaneously (s.c.) such as tissue ipsilateral (i.l.) to the tumor and tissue contralateral (c.l.) to the tumor.

As used herein, an “admixture” is a simple combination of two or more components, wherein the components are not covalently or otherwise irreversibly linked to one another.

As used herein, “antibody” refers to a glycoprotein immunoglobulin which specifically binds to an antigen and comprises at least two light and two heavy chains interconnected by disulfide bonds. The antibody is composed of a variable region and a constant region where the variable region recognizes distinct antigens, and the constant region is recognized by other cells of the immune system and components of the complement system.

As used herein, “monoclonal antibody” (mAb) refers to a non-naturally occurring antibody where the primary sequences are identical leading to a single binding specificity and affinity to a particular epitope. mAbs may be produced by hybridoma, recombinant, transgenic or other techniques.

As used herein, “cytotoxic T-lymphocyte antigen 4” (CTLA-4) refers to a transmembrane immunoinhibitory receptor found on a variety of T cells, B cells, dendritic cells, (DCs), macrophages, myeloid-derived suppressor cells (MDSCs), and cancer cells. CTLA-4 binds to two ligands, CD80 and CD86, and has opposing function to CD28. It prevents T cells activation.

As used herein, “programmed death-1” (PD-1) refers to a transmembrane receptor found primarily on activated T cells with two ligands, PD-L1 and PD-L2. PD-1 restricts the function of activated T cells including the cytotoxic function.

As used herein, the term “programmed death ligand-1” (PD-L1) and “Programmed death ligand-1” (PD-L1)” refers to each one of the two surface ligands to PD-1 and is found on a variety of hematopoietic and nonhematopoietic cells such as antigen-presenting cells, MDSCs, and cancer cells. PD-L1 and PD-L2 on antigen-presenting cells lead to suppress T cell activation and function following binding to PD-1 on T cells. PD-L1 and PD-L2 on cancer cells facilitate the bypass of immune surveillance following binding to PD-1 on T cells.

As used herein, “lymphocyte-activation gene 3” (LAG-3) refers to a cell surface immune checkpoint receptor molecule that is expressed on activated T cells, NK cells, B cells and dendritic cells. The interaction of LAG-3 with MHCII on antigen-presenting cells suppresses the activations of T cells.

As used herein, “T cell immunoglobulin and mucin-domain containing-3” (TIM-3) refers to a cell surface immune checkpoint receptor molecule expressed on T cells, dendritic cells, and macrophages, which mediates CD8 T cell exhaustion and innate functions of dendritic cells, macrophages and NK cells. TIM-3 expressed on DCs competes with the HMGB1 in binding with nucleic acids released from dying tumor cells, which effectively attenuates the activation of innate immune response. Binding on TIM-3 with galectin-9 induce apoptosis of Th1, which suppress the antitumor immune response. Binding of TIM-3 with phosphatidylserine (PtdSer) exposed on the surface of apoptotic cells promotes the cross-presentation of antigens by dendritic cells. Up-regulation of TIM-3 represents the exhaustion of CD8 T cells.

As used in herein, “T cell immunoglobulin and ITIM domain (TIGIT)” refers to a cell surface immune checkpoint receptor molecule on NK cells and exhausted T cells, which mediates CD8 T cell exhaustion and NK cell functions. The interaction of TIGIT with CD155 on antigen-presenting cells suppresses the activations of T cells and NK cells, while CD226 on T cells and NK cells binds to CD155 on antigen-presenting cells leads to the activation of T cells and NK cells.

As used herein, “immune checkpoint blockade” or “immune checkpoint blockade antibody” or “ICB” or “immune checkpoint inhibitor” refers to a monoclonal antibody that binds and modulates immune checkpoint including, but not limited to, CTLA-4, PD-1, PD-L1, LAG-3, TIM-3, and TIGIT, to agonistically or antagonistically. A “a” prefix to each immune checkpoint refers to the antibody of each immune checkpoint; aCTLA-4, aPD-1, aPD-L1, aLAG-3, aTIM-3, aTIGIT and so on.

As used herein, the term “chemotherapeutic drugs” or “anticancer chemotherapeutic drugs” refers to a chemical agent that stops the growth of cancer cells by killing the cells or by stopping the proliferation, division, and differentiation of the cells. Although the primary purpose of chemotherapeutic drugs is to kill the cancer cells and stop the proliferation, division, and differentiation of the cancer cells by directly affecting the cancer cells, they can also directly and indirectly modulate the immune response, which governs the tumor growth.

As used herein, “lymph node” (LN) refers to a bean shaped structure that house the body's immune system which are scattered throughout the body. LNs filter foreign substances that travel through the lymphatic fluid and contain various immune cells. LNs are where lymphocytes are activated against specific antigens. Tumor draining lymph nodes lie immediately downstream of tumors and undergo alterations in their structure and function, which is due to the drainage of tumor antigens and signaling molecules from the presence of the upstream tumor.

As used herein, a “micelle” refers to an aggregate of surfactant containing hydrophilic shell and hydrophobic core, which facilitates the drug delivery by physically encapsulating hydrophobic drugs into the hydrophobic core of the micelles, by physically absorbing hydrophilic drugs onto the hydrophilic surfaces, or by electrostatically absorbing or chemically conjugating any types of pharmaceutical drugs and diagnostic agents, such as hydrophobic chemical drugs, hydrophilic chemical drugs, ICBs, protein drugs, peptide drugs, NO-donors, nucleotides, and imaging agents.

As used herein, “in-situ micelles” refers to micelles formed in-situ from polymers forming the macroscopic biomedical devices or DDSs when released or degraded from the macroscopic biomedical devices or DDSs including microneedle, scaffold, and hydrogels.

As used herein, “lymphatics” or “lymph vessels” refer to a part of the lymphatic system that transport lymph in the body. Lymphatics are organized as one-way vessels that help absorb interstitial fluid known as lymph from tissues and transport it to lymph nodes.

As used herein, “immune response” refers to the action of the immune system including immune cells and macromolecules produced by these cells that leads selective targeting and destruction of pathogens or cancer cells and healthy cells in the case of autoimmunity.

As used herein, “checkpoint blockade therapy” refers to the inhibition of CTLA-4 and/or PD-1 and/or PD-L1 and/or LAG-3 and or TIM-3 and/or TIGIT as well as other immune checkpoint pathways.

As used herein, “local” refers to an administration that is in tumor and in the lymphatic tissue basin of which drains to the lymph node.

As used herein, “T cell” refers to a lymphocyte produced by the thymus gland that resides in lymph nodes. T cells play a major role in cell-mediated immunity which is mediated by their specificity toward antigens due to their T cell receptor (TCR) and cytotoxic mechanisms to eliminate infected or mutated cells. T cells play a major role in cancer immunotherapy and express CTLA-4, PD-1, LAG-3, TIGIT.

As used herein, “antigen-presenting cell (APC)” refers to cells to display antigens complexed with major histocompatibility complexes (MHCs) on their surfaces, which includes, but not limited to, dendritic cells, macrophages, and B cells.

As used herein, “dendritic cell (DC)” refers to a dendritic-shaped immune cell to be primarily responsible for the initiation of adaptive immune response by processing and presenting antigens to their surface to prime and instruct T cells. DCs are also responsible for an innate immune response via phagocytosis and cytokines release.

As used herein, “macrophage” refers to a specialized cell primarily for detection, phagocytosis and destruction of foreign materials. Macrophages also act as an APCs by priming and instructing T cells.

As used herein, “B cell” refers a type of white blood cell to be primarily responsible for the humoral immunity by producing antigen-specific antibodies. B cells are also responsible for an innate immune response via phagocytosis and cytokines release.

As used herein, “natural killer cell (NK cell)” refers a type of white blood cell to be primarily responsible for the innate immune response, which has small granules containing various enzymes to exert toxicity to the cancer cells, bacteria or virus.

As used herein, “natural killer T cell (NKT cell)” refers a type of T cells expressing both TCR and specific NK cell markers. Therefore, NKT shares functions of both T cells and NK cells.

As used herein, “myeloid-derived suppressor cells (MDSCs)” refers to heterogenous immune cells from myeloid lineage, which exert immunosuppressive activities to regulate T cells, DCs, macrophages and NK cells.

Image guided surgery is the surgical procedure where surgeons utilize intraoperative images during surgery in real-time by using intraoperative imaging machines sometimes supported with imaging agents.

Near infrared light (NIR) is defined to have wavelength from 700 nm to 2000 nm, which has lower absorption and scattering on tissues and biomolecules than visible light when it is irradiated to the animals and humans.

NIR imaging agent is defined as a fluorescent dye, nanoparticle, and polymer that absorb NIR light and then emit the NIR light. NIR imaging agents include not only indocyanin green and methylene blue that are approved by FDA and EMA, but also numerous organic and inorganic small molecules, nanoparticles, and polymers to absorb and emit NIR light.

A polypeptide refers to a polymer composed of amino acid monomers, linked together via amide bonds. The amino acids can be naturally occurring amino acids, unnaturally occurring amino acids, and combinations thereof. The amino acids can be a-amino acids, (β-amino acids, γ-amino acids, or δ-amino acids. Unless specified to the contrary, amino acids, both generally and in regard to specific amino acids, should be understood to have the (L) configuration as found in naturally occurring amino acids.

As used herein, “hydroxyproline” refers to the compound (2S,4R)-4-hydroxypyrrolidine-2-carboxylic acid.

Many of the compounds and compositions disclosed herein include ionizable atoms, e.g., basic nitrogen atom, carboxylic acid groups, etc. The skilled person understands that whether a particular group is ionized or not depends on the local chemical environment including pH. Unless specified explicitly to the contrary, the depiction of any atom or functional group in one ionized (or non-ionized) state includes all possible ionization states for said atom or functional group.

Unless specified otherwise, the term “patient” or “subject” refers to any mammalian animal, including but not limited to, humans.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. Pharmaceutically acceptable salts are salts that retain the desired biological activity of the parent compound and do not impart undesirable toxicological effects. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate. Pharmaceutically acceptable and non-pharmaceutically acceptable salts may be prepared using procedures well known in the art, for example, by reacting a sufficiently basic compound such as an amine with a suitable acid comprising a physiologically acceptable anion. Alkali metal (for example, sodium, potassium, or lithium) or alkaline earth metal (for example, calcium) salts of carboxylic acids can also be made. Lists of additional suitable salts may be found, e.g., in Remington's Pharmaceutical Sciences, 17th ed., Mack Publishing Company, Easton, Pa., p. 1418 (1985).

Disclosed herein are thermosensitive hydrogels. In some embodiments the thermosensitive hydrogels can be used in drug delivery systems. The thermosensitive hydrogels can be a LCST hydrogel, meaning they exist as a fluid or a sol in an aqueous dispersion medium at lower temperatures, but undergo a phase transition above the critical temperature, thereby forming a hydrogel. In some instances, the thermosensitive hydrogels can have a sol-to-gel transition temperature from about 10-40° C., from about 15-40° C., from about 15-37° C., from about 15-35° C., from about 15-30° C., from about 15-25° C., from about 20-37° C., from about 21-37° C., from about 22-37° C., from about 23-37° C., from about 24-36° C., from about 25-35° C., from about 26-34° C., from about 27-33° C., or from about 28-32° C. In some embodiments the thermosensitive hydrogels are fluid/sol at room temperature (−23° C.), and a gel above about 33° C. (for instance above about 37° C.), undergoes a phase transition into a hydrogel. In several aspects of the disclosure, the thermosensitive hydrogel is provided in a composition containing water, wherein the hydrogel has a certain concentration, usually expressed as a weight fraction. Unless specified explicitly to the contrary, the weight fraction can be used to describe both solution and gel forms of the hydrogel.

The thermosensitive hydrogel can include one or more thermosensitive polymers conjugated to one or more polypeptides, thermosensitive biopolymer, and/or thermosensitive polypeptide. The thermosensitive polymer can be a LCST (sol-to-gel) polymer, while the thermosensitive biopolymer or thermosensitive polypeptide can be a UCST (gel-to-sol) biopolymer or peptide. The thermosensitive polymer can be a linear polymer, e.g., a polymer having a first end, or first terminus, and a second end, or second terminus. In other embodiments, the thermosensitive polymer is a branched polymer, having more than two termini, e.g., three termini, four termini, five termini, and the like. In some embodiments of the thermosensitive hydrogel the thermosensitive polymers are covalently crosslinked, while in other embodiments the thermosensitive polymers are not covalently crosslinked. A covalently crosslinked hydrogel is one in which at least a portion of the thermosensitive polymers are covalently conjugated to the polypeptide through at least two termini of the thermosensitive polymer. A singly linked thermosensitive polymer is one in which one terminus of the polymer is covalently conjugated to a polypeptide while the other terminus (or termini) is not covalently conjugated to a polypeptide. In certain embodiments, the portion of thermosensitive polymers covalently conjugated to a polypeptide through at least two termini is at least 15%, at least 25%, at least 50%, at least 60%, at least 70%, at least 80%, at least 85%, at least 90%, or at least 95%, relative to the total amount of thermosensitive polymer in the hydrogel. In some embodiments, the portion of thermosensitive polymers covalently conjugated to a polypeptide through at least two termini is no more than 15%, no more than 25%, no more than 50%, no more than 60%, no more than 70%, no more than 80%, no more than 85%, no more than 90%, or no more than 95%, relative to the total amount of thermosensitive polymer in the hydrogel. In further embodiments, the portion of thermosensitive polymers covalently conjugated to a polypeptide through at least two termini is from 15-50%, from 25-75%, from 50-95%, from 20-60%, from 30-70%, from 40-80%, from 50-85%, from 60-90%, or from 75-95%, relative to the total amount of thermosensitive polymer in the hydrogel.

In some embodiments, the thermosensitive polymer is a linear block co-polymer at least one polyethylene glycol (“PEG”) block and at least one block of one other polymer, for instance polypropylene glycol (“PPG”), a polycarbonate, or a polyester like polylactic acid (“PLA”), poly(lactic-co-glycolic acid (“PLGA”), poly(3-hydroxybutyrate) (“PHB”), polycaprolactone (“PCL”). The block copolymer can be a di-block copolymer (i.e., two separate domains), or a tri-block copolymer (i.e., three separate domains). In some embodiments, the block copolymer can have 4, 5, 6, 7, or 8 separate domains. In some instances the block copolymer can be a triblock polymer that have a single block of polyethylene glycol and two blocks of a polypropylene glycol, polycarbonate or polyester ([PPG/polycarbonate/polyester]-[PEG]-[PPG/polycarbonate/polyester]). In other instances the block copolymer can be a triblock polymer that have two blocks of polyethylene glycol and a single block of a polypropylene glycol, polycarbonate or polyester ([PEG]-[PPG/polycarbonate/polyester]-[PEG]).

Exemplary thermosensitive polymers include polymers sold under the name Tetronic® (T304, T904, and T1307), or Pluronic® (F127, P85, and F68). In some embodiments the thermosensitive polymer has the general formula: PEG-PPG-PEG, PPG-PEG-PPG, PPG-PEG, PLGA-PEG-PLGA, PEG-PLGA-PEG, PEG-PLGA, PEG-PLA-PEG, PLA-PEG-PLA, PEG-PLA, PCL-PEG-PCL, PEG-PCL-PEG, PEG-PCL, PHB-PEG-PHB, PEG-PHB-PEG, or PEG-PHB.

Exemplary branched thermosensitive polymers include those having the general formula [Core]-[PPG-PEG]ₓ, [Core]-[PEG-PLGA]ₓ, [Core]-[PLGA-PEG]ₓ, [Core]-[PLA-PEG]ₓ, [Core][PEG-PLA]ₓ, [Core]-[PCL-PEG]ₓ, [Core][PEG-PHB]ₓ, [Core][PHB-PEG]ₓ, wherein x is 3, 4, 5, 6, 7, or 8, preferably 3 or 4, and [Core] has the formula:

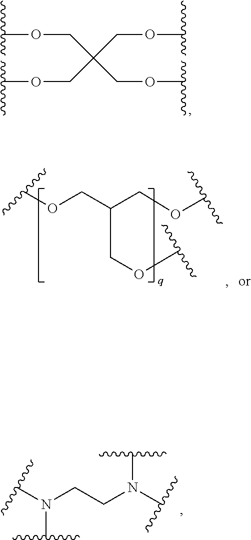

wherein q is 1, 2, 3, 4, 5, or 6, preferably 1 or 2, and each wavy line represents a bond to a thermosensitive polymer chain.

The thermosensitive block copolymer (whether linear or branched) can have an average molecular weight from 2,500-100,000, from 2,500-75,000, from 2,500-50,000, from 2,500-30,000, 2,500-25,000, from 2,500-20,000, from 2,500-15,000, from 2,500-10,000, from 2,500-5,000, from 50,000-100,00 from 50,000-75,000, from 25,000-75,000, from 25,000-50,000, from 10,000-50,000, from 10,000-25,000, from 5,000-25,000, from 5,000-20,000, from 5,000-15,000, or from 7,500-15,000.

In other embodiments, the thermosensitive polymer is a polyacrylamide, e.g., poly(N-isopropylacrylamide), poly(N-vinylisobutyramide), or poly(acrylamide), a cellulose like methylcellulose, hydroxypropyl methylcellulose, carboxymethylcellulose, or a naturally occurring polymer like chitosan, collagen, or hyaluronic acid.

In some instances, the thermosensitive hydrogel includes a thermosensitive biopolymer. The thermosensitive biopolymer can have an upper critical solution temperature, in which the aqueous compositions of the thermosensitive biopolymer are a sol or fluid at higher temperatures and a gel at lower temperatures. In some instances, thermosensitive biopolymer can undergo gel-to-sol transition at a temperature from about 30-50° C., from about 35-50° C., from about 40-50° C., from about 30-45° C., from about 30-40° C., or from about 35-45° C. In some embodiments, the thermosensitive biopolymer is a polypeptide. In certain embodiments, the polypeptide has an average molecular weight from 15,000-150,000, from 20,000-50,000, from 20,000-30,000, from 30,000-75,000, from 40,000-60,000, from 40,000-50,000, from 50,000-150,000, from 50,000-125,000, or from 50,000-100,000.

The thermosensitive polypeptide can include glycine, proline and hydroxyproline residues. In some embodiments, the polypeptide can include glycine in an amount from 20-40% relative to the total number of amino acids in the polypeptide, proline in an amount from 7.5-22.5% relative to the total number of amino acids in the polypeptide, and hydroxyproline in an amount from 5-20% relative to the total number of amino acids in the polypeptide. The polypeptide can be characterized by an abundance of glycine, proline, and hydroxyproline. For example, the total content of these three amino acids can be at least 25%, at least 30%, at least 35%, at least 40%, at least 45%, at least 55%, or at least 60% relative to the total number of amino acids in the polypeptide. Without wishing to be bound by theory, it is believed the abundance of these amino acid residues contribute to the gelation of the thermosensitive hydrogel. The polypeptide may also include arginine in an amount from 5-25 20%, from 5-15%, or from 5-10% relative to the total number of amino acids in the polypeptide. The polypeptide may include aspartic acid in an amount of from 3-12%, from 3-9%, or from 5-9% relative to the total number of amino acids in the polypeptide. The polypeptide may include glutamic acid in an amount from 5-20%, from 5-15%, or from 5-10% relative to the total number of amino acids in the polypeptide.

In some embodiments, the polypeptide includes a gelatin. In some instances, the polypeptide may include a mixture of gelatin and at least one other polypeptide, while in other embodiments, gelatin is the sole polypeptide in the thermosensitive hydrogel. Gelatin is a polypeptide containing an abundance of glycine, proline, and hydroxyproline residues. Gelatin is obtained by partially hydrolyzing collagen. The thermosensitive hydrogels can include gelatin having a variety of Bloom values. For example, the gelatin can be high Bloom (Bloom number from 225 to 325), medium Bloom (Bloom number from 175 to 225) or low Bloom (Bloom number 50 to 125). High Bloom gelatin generally contains higher molecular weight polypeptide chains relative to low Bloom gelatin. Bloom is determined by the mass (in grams) required to depress a plunger (diameter=0.5 inch) 4 mm into the surface of the gel. Low Bloom gelatin has an average molecular mass of 20,000-25,000. Medium Bloom gelatin has an average molecular mass of 40,000-50,000. High Bloom gelatin has an average molecular mass of 50,000-100,000. The gelatin can be type A, which is obtained by acid catalyzed hydrolysis, or the gelatin can be type B, which is obtained by base (i.e., lime) catalyzed hydrolysis.

The thermosensitive hydrogels may include one or more thermosensitive polymers conjugated to one or more polypeptides. Preferably, the conjugation may be through covalent bonds, although in certain embodiments, the thermosensitive polymers may be conjugated to the polypeptides via non-covalent bonds (e.g., ionic bonds, hydrogen bonds, Van der Waals interactions, and combinations thereof).

The thermosensitive hydrogels may be provided in the dehydrated state, for example including no more than 10%, no more than 7.5%, no more than 5%, no more than 4%, no more than 3%, no more than 1%, or no more than 1% of water by weight, relative to the entire weight of the dehydrated hydrogel. Dehydrated hydrogels may be rehydrated by contacting the dehydrated hydrogel with water, optionally in an aqueous solution containing one or more therapeutic agents or excipients as described herein.

When hydrated, the thermosensitive hydrogel compositions can contain from 70-99.99% by weight of water, relative to the weight of the total composition. In some embodiments, the hydrogel compositions can contain from 75-99.99%, from 80-99.99%, from 85-99.99%, from 90-99.99%, from 92.5-99.99%, from 95-99.99%, from 97.5-99.99%, from 85-98%, from 90-98%, from 92.5-98%, from 95-98%, from 97.5-98%, from 90-96%, from 92.5-96%, from 95-96%, from 92.5-97.5%, from 93-97%, or from 94-97% by weight of water, relative to the weight of the total composition.

The thermosensitive polymers (prior to conjugation to polypeptide) can be characterized by a reactive functional group at one or more termini. For example, native PEG and PPG have the same functional group (a hydroxyl) at each terminus, while polyesters like PLA, PLGA, PCL, and PHB are characterized by different functional groups at each terminus, i.e., a carboxylate at one terminus, and a hydroxyl group at the other. These different functional groups can be exploited to control the degree of crosslinking conjugation vs. single conjugation. Additionally, these polymers can be modified using conventional techniques such that both termini have the same functional group, for example esterification of a carboxylate with ethylene glycol, or acylation of a hydroxyl group with an anhydride or activated carboxylate such as succinic anhydride. In other embodiments, amino groups (including amino acids), thiols, Michael acceptors, and olefins may be installed at one or more termini of the thermosensitive polymer using known techniques. Exemplary Michael acceptors include maleimides, α,(β-unsaturated ketones, esters, and sulfones.

The thermosensitive hydrogels can be composed of the thermosensitive polymer and polypeptide in various weight ratios. In some embodiments the ratio of thermosensitive polymer:polypeptide in the thermosensitive hydrogel can be from 5:1 to 1:5 wt./wt., 4:1 to 1:4 wt./wt., 3:1 to 1:3 wt./wt., from 2:1 to 1:2 wt./wt., from 1.5:1 to 1:1.5 wt./wt., from 5:1 to 1:1 wt./wt., from 4:1 to 1:1 wt./wt., from 3:1 to 1:1 wt./wt., from 2:1 to 1:1 wt./wt., from 1.5:1 to 1:1 wt./wt., from 1:1 to 1:1.5 wt./wt., from 1:1 to 1:2 wt./wt., from 1:1 to 1:3 wt./wt., 1:1 to 1:4 wt./wt., or from 1:1 to 1:5 wt./wt. As used herein, a given weight ratio in a thermosensitive hydrogel is the same as the weight ratio of the thermosensitive polymer and polypeptide used to prepare the hydrogel. If either of the thermosensitive polymer or polypeptide is modified with an activating group prior to conjugation, the weight ratio is calculated based on the weight of the thermosensitive polymer or polypeptide prior to reaction with the activating group.

In certain embodiments, the thermosensitive hydrogel can include the polypeptide component in an amount from 15-85%, from 50-85%, from 25-85%, from 25-75%, from 35-75%, from 35-65%, from 40-60%,from 10-25%, 15-25%, from 15-35%, from 20-30%, from 20-40%, from 20-45%, from 25-40%, from 30-40%, from 30-45%, from 35-45%, or from 40-50% by weight, relative to the total weight of the polypeptide +thermosensitive polymer in thermosensitive hydrogel.

In some preferred embodiments, the thermosensitive polymer is a block copolymer having the formula PEG-PPG-PPG, and having an average molecular weight from 5,000-20,000, and the polypeptide is gelatin, having a Bloom number from 225 to 325. The polypeptide and gelatin may be combined such that the gelatin is present in an amount from 15-85%, from 50-85%, from 25-85%, from 25-75%, from 35-75%, from 35-65%, from 40-60%, from 10-25%, 15-25%, from 15-35%, from 20-30%, from 20-40%, from 20-45%, from 25-40%, from 30-40%, from 30-45%, from 35-45%, or from 40-50% by weight, relative to the total weight of the polypeptide+thermosensitive polymer in thermosensitive hydrogel. In some preferred embodiments, the gelatin is present in an amount from 25-50% by weight, relative to the total weight of the thermosensitive hydrogel.

Chemical conjugation between thermosensitive polymers and polypetides can be performed by directly conjugating amine groups of the polypeptide to the hydroxyl groups or carboxylic acid groups of thermosensitive polymers. Such conjugations may be through a urethane (or carbamate) bond. Typically, the thermosensitive polymer is first combined with an activating group, such as 4-nitrophenyl chloroformate, carbonyldiimidazole, N-hydroxysuccinimide, or carbodiimide. In other embodiments the thermosensitive polymer includes an amino group at one or more termini, which is directly conjugated to the hydroxyl groups or carboxylic acid groups in the polypeptide using similar chemistries. In other embodiments, amino groups in the thermosensitive polymer can be conjugated to amino groups in the polypeptide using genipin, glutaraldehyde, disuccinimidyl suberate (DSS) or bis(sulfosuccinimidyl) subberate (BS3). In other embodiments, thermosensitive polymers containing thiol groups can be conjugated to amine groups in the polypeptide using Michael acceptor groups, to polypeptides containing alkene groups via thiol-ene reactions. Thermosensitive polymers containing alkenes can be conjugated to polypeptides containing alkene groups via metathesis reactions. The thermosensitive polymer and polypeptide may be conjugated using click chemistries, e.g., the cycloaddition reaction between either 1,3 dipole (e.g., azide, nitrone) or 1,2,4,5-tetrazine (or more simply, tetrazine) and an alkyne or alkene, for instance a strained cyclooctyne or trans-cyclooctene. One or more termini in the thermosensitive polymer can be functionalized with one click component (e.g., either an azide/tetrazine or strained cyclooctyne/trans-cyclooctene).

In some embodiments, the thermosensitive polymer and polypeptide are conjugated using succinimidyl-4-(N-maleimidomethyl)cyclohexane-1-carboxy-(6-amidocaproate) (LC-SMCC), sulfosuccinimidyl 4-[N-maleimidomethyl]cyclohexane-1-carboxylate (Sulfo-SMCC), succinimidyl 6-[3(2-pyridyldithio)propionamido]hexanoate (LC-SPDP), sulfosuccinimidyl 6-[3′-(2-pyridyldithio)propionamido]hexanoate (sulfo-LC-SPDP), succinimidyl-6((β-maleimidopropionamido)hexanoate (SMPH), 3-(2-pyridyldithio) propionic acid N-hydroxysuccinimide ester, 3-(Maleimido)propionic acid N-hydroxysuccinimide ester, 4-maleimidobutyric acid N-hydroxysuccinimide ester, 6-maleimidohexanoic acid N-hydroxysuccinimide ester, PEG-SPDP, or heterobifunctional PEGs with maleimide and NHS groups. Exemplary click activating groups include N-[(1R,8S,9s)-Bicyclo[6.1.0]non-4-yn-9-ylmethyloxycarbonyl]-1,8-diamino-3,6-dioxaoctane, (1R,8S,9s)-bicyclo[6.1.0]non-4-yn-9-ylmethanol, (1R,8S,9s)-bicyclo[6.1.0]non-4-yn-9-ylmethyl N-succinimidyl carbonate, dibenzocyclooctyne-amine, dibenzocyclooctyne-N-hydroxysuccinimidyl ester, dibenzocyclooctyne-S-S-N-hydroxysuccinimidyl ester, dibenzocyclooctyne-maleimide, dibenzocyclooctyne-sulfo-N-hydroxysuccinimidyl ester, and trans-cycloocten-4-ol.

In certain embodiments, the thermosensitive polymer may be depicted as in FIG. 1, showing a polypeptide backbone having multiple COOH (for example from glutamic acid and aspartic acid residues) and NH₂ groups (from lysine or arginine residues, also included, but not depicted are, asparagine, glutamine, and histidine residues (having an NH instead of NH₂ group)), wherein the thermosensitive polymer PEG-PPG-PEG is first activated with a molar excess of carboxyl donor (e.g., 4-nitrophenyl chloroformate), and then reacted with the amine groups of gelatin to give thermosensitive hydrogel. The depicted hydrogel include a portion of crosslinked thermosensitive polymers and a portion of single linked thermosensitive polymers. In certain preferred embodiments, the thermosensitive polymer may be functionalized with a Michael acceptor (e.g., vinyl sulfone, maleimide, unsaturated ketone or ester) and conjugated to reactive amines/thiols in the polypeptide.

In the embodiments, the thermosensitive hydrogel network can include a portion of crosslinked thermosensitive polymers having the formula:

[PP]—X^{aa}—[L¹]—[CH₂]_{z}—[TP]—[CH₂]_{z}—[L²]—X^{aa}—[PP]

wherein:
TP represents a thermosensitive polymer as defined herein;
PP represents a polypeptide as defined herein, preferably gelatin, even more preferably gelatin having a Bloom number from 225 to 325;
X^{aa} is in case N, NH, S, O, C(O);

### Z is 0, 1, or 2

L¹ is a linker groups selected from the following:

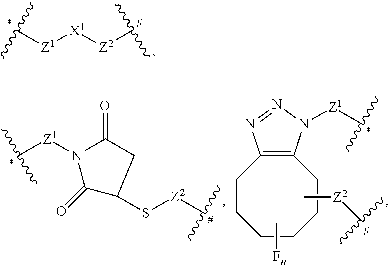

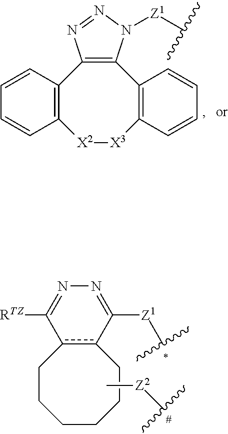

R^{TZ} is H, CH₃, or phenyl;
one of * or # represents the point of attachment to PP, and the other of * or # represents the point of attachment to —[CH₂]_{z}—;
X¹ is C═O, or SO₂,
one of X² and X³ is N—Z²—^{#} or —CH—Z²—^{#}; and the other of X² and X³ is CH₂ or C═O; or X² and X³ together represent a group having the formula:

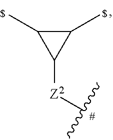

wherein each $ represents a point of attachment to the remainder of the cyclooctyne;
Z¹ is selected from null, —Z^{1a}—A^{1a}—Z^{1b}—A^{1c}—Z^{1d}—;
A^{1a} is selected from null, C₁₋₁₀alkylene, —(CH₂CH₂O)_{zaa}CH₂CH₂—, C₃₋₁₀cycloalkylene, C₆₋₁₀arylene, C₁₋₁₀aheterocyclylene, or C₁₋₁₀aheteroarylene; preferably A^{1a} is selected from null, C₁₋₁₀alkylene, or —(CH₂CH₂O)_{zaa}CH₂CH₂—;
A^{1b} is selected from null, C₁₋₁₀alkylene, —(CH₂CH₂O)_{zab}CH₂CH₂—,C₃₋₁₀cycloalkylene, C₆₋₁₀arylene, C₁₋₁₀aheterocyclylene, or C₁₋₁₀aheteroarylene; preferably A^{1b} is selected from null, C₁₋₁₀alkylene, or —(CH₂CH₂O)_{zab}CH₂CH₂—;
A^{1c} is selected from null, C₁₋₁₀alkylene, —(CH₂CH₂O)_{zac}CH₂CH₂—, C₃₋₁₀cycloalkylene, C₆₋₁₀arylene, C₁₋₁₀aheterocyclylene, or C₁₋₁₀aheteroarylene; preferably A^{1c} is selected from null, C₁₋₁₀alkylene, or —(CH₂CH₂O)_{zac}CH₂CH₂—;
Z^{1a} is selected from null, —NH—, —O—, —S—, —NH(CO), —C(O)NH—, —O(CO)—, —(CO)O—, —NH(CO)O—, —OC(O)NH—, —NH(CO)NH,
Z^{1b} is selected from null, —NH—, —O—, —S—, —NH(CO), —C(O)NH—, —O(CO)—, —(CO)O—, —NH(CO)O—, —OC(O)NH—, —NH(CO)NH,
Z^{1c} is selected from null, —NH—, —O—, —S—, —NH(CO), —C(O)NH—, —O(CO)—, —(CO)—, —NH(CO)O—, —OC(O)NH—, —NH(CO)NH,
Z^{1d} is selected from null, —NH—, —O—, —S—, —NH(CO), —C(O)NH—, —O(CO)—, —(CO)—, —NH(CO)O—, —OC(O)NH—, —NH(CO)NH,
zaa is 1, 2, 3, 4, 5, 6, 7, 8, 9, 10, 11, 12, 13, 14, 15, 16, 17, 18, 19, or 20;
zab is 1, 2, 3, 4, 5, 6, 7, 8, 9, 10, 11, 12, 13, 14, 15, 16, 17, 18, 19, or 20; and
zac is 1, 2, 3, 4, 5, 6, 7, 8, 9, 10, 11, 12, 13, 14, 15, 16, 17, 18, 19, or 20;
Z² is selected from null, —Z^{2a}—A^{2a}—Z^{2b}—A^{2b}—Z^{2c}—A^{2c}—Z^{2d}—,
A^{2a} is selected from null, C₁₋₁₀alkylene, —(CH₂CH₂O)_{zba}CH₂CH₂—, C₃₋₁₀cycloalkylene, C₆₋₁₀arylene, C₁₋₁₀aheterocyclylene, or C₁₋₁₀aheteroarylene; preferably A^{2a} is selected from null, C₁₋₁₀alkylene, or —(CH₂CH₂O)_{zba}CH₂CH₂—;
A^{2b} is selected from null, C₁₋₁₀alkylene, —(CH₂CH₂O)_{zbb}CH₂CH₂—,C₃₋₁₀cycloalkylene, C₆₋₁₀arylene, C₁₋₁₀aheterocyclylene, or C₁₋₁₀aheteroarylene; preferably A^{2b} is selected from null, C₁₋₁₀alkylene, or —(CH₂CH₂O)_{zbb}CH₂CH₂—;
A^{2c} is selected from null, C₁₋₁₀alkylene, —(CH₂CH₂O)_{zcc}CH₂CH₂—, C₃₋₁₀cycloalkylene, C₆₋₁₀arylene, C₁₋₁₀aheterocyclylene, or C₁₋₁₀aheteroarylene; preferably A^{2c} is selected from null, C₁₋₁₀alkylene, or —(CH₂CH₂O)_{zbc}CH₂CH₂—;
Z^{2a} is selected from null, —NH—, —O—, —S—, —NH(CO), —C(O)NH—, —O(CO)—, —(CO)O—, —NH(CO)O—, —OC(O)NH—, —NH(CO)NH,
Z^{2b} is selected from null, —NH—, —O—, —S—, —NH(CO), —C(O)NH—, —O(CO)—, —(CO)O—, —NH(CO)O—, —OC(O)NH—, —NH(CO)NH,
Z^{2c} is selected from null, —NH—, —O—, —S—, —NH(CO), —C(O)NH—, —O(CO)—, —(CO)—, —NH(CO)O—, —OC(O)NH—, —NH(CO)NH,
Z^{2d} is selected from null, —NH—, —O—, —S—, —NH(CO), —C(O)NH—, —O(CO)—, —(CO)—, —NH(CO)O—, —OC(O)NH—, —NH(CO)NH,
zba is 1, 2, 3, 4, 5, 6, 7, 8, 9, 10, 11, 12, 13, 14, 15, 16, 17, 18, 19, or 20;
zbb is 1, 2, 3, 4, 5, 6, 7, 8, 9, 10, 11, 12, 13, 14, 15, 16, 17, 18, 19, or 20; and
zbc is 1, 2, 3, 4, 5, 6, 7, 8, 9, 10, 11, 12, 13, 14, 15, 16, 17, 18, 19, or 20;
L² is a linker groups selected from the following:

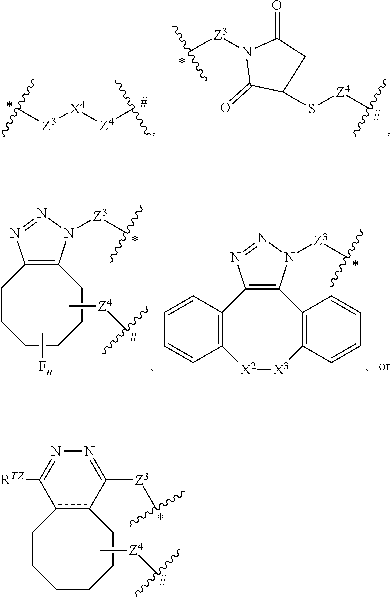

R^{TZ} is H, CH₃, or phenyl;
one of * or # represents the point of attachment to PP, and the other of * or # represents the point of attachment to —[CH₂]_{z}—;
one of X² and X³ is N—Z⁴—^{#} or —CH—Z⁴—^{#} ; and the other of X² and X³ is CH₂ or C═O; or X² and X³ together represent a group having the formula:

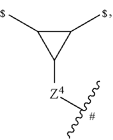

wherein each $ represents a point of attachment to the remainder of the cyclooctyne; X⁴ is C═O, or SO₂,
Z³ is selected from null, —Z^{3a}—A^{3a}—Z^{3b}—A^{3b}—Z^{3c}—A^{3c}—Z^{3d}—,
A^{3a} is selected from null, C₁₋₁₀alkylene, —(CH₂CH₂O)_{zca}CH₂CH₂—, C₃₋₁₀cycloalkylene, C₆₋₁₀arylene, C₁₋₁₀aheterocyclylene, or C₁₋₁₀aheteroarylene; preferably A^{3a} is selected from null, C₁₋₁₀alkylene, or —(CH₂CH₂O)_{zca}CH₂CH₂—;
A^{3b} is selected from null, C₁₋₁₀alkylene, —(CH₂CH₂O)_{zcb}CH₂CH₂—,C₃₋₁₀cycloalkylene, C₆₋₁₀arylene, C₁₋₁₀aheterocyclylene, or C₁₋₁₀aheteroarylene; preferably A^{3b} is selected from null, C₁₋₁₀alkylene, or —(CH₂CH₂O)_{zcb}CH₂CH₂—;
A^{3c} is selected from null, C₁₋₁₀alkylene, —(CH₂CH₂O)_{zcc}CH₂CH₂—, C₃₋₁₀cycloalkylene, C₆₋₁₀arylene, C₁₋₁₀aheterocyclylene, or C₁₋₁₀aheteroarylene; preferably A^{3c} is selected from null, C₁₋₁₀alkylene, or —(CH₂CH₂O)_{zcc}CH₂CH₂—;
Z^{3a} is selected from null, —NH—, —O—, —S—, —NH(CO), —C(O)NH—, —O(CO)—, —(CO)O—, —NH(CO)O—, —OC(O)NH—, —NH(CO)NH,
Z^{3b} is selected from null, —NH—, —O—, —S—, —NH(CO), —C(O)NH—, —O(CO)—, —(CO)O—, —NH(CO)O—, —OC(O)NH—, —NH(CO)NH,
Z^{3c} is selected from null, —NH—, —O—, —S—, —NH(CO), —C(O)NH—, —O(CO)—, —(CO)—, —NH(CO)O—, —OC(O)NH—, —NH(CO)NH,
Z^{3d} is selected from null, —NH—, —O—, —S—, —NH(CO), —C(O)NH—, —O(CO)—, —(CO)—, —NH(CO)O—, —OC(O)NH—, —NH(CO)NH,
zca is 1, 2, 3, 4, 5, 6, 7, 8, 9, 10, 11, 12, 13, 14, 15, 16, 17, 18, 19, or 20;
zcb is 1, 2, 3, 4, 5, 6, 7, 8, 9, 10, 11, 12, 13, 14, 15, 16, 17, 18, 19, or 20;
zcc is 1, 2, 3, 4, 5, 6, 7, 8, 9, 10, 11, 12, 13, 14, 15, 16, 17, 18, 19, or 20;
Z⁴ is selected from null, —Z^{4a}—A^{4a}—Z^{4b}—A^{4b}—Z^{4c}—A^{4c}—Z^{4d}—,
A^{4a} is selected from null, C₁₋₁₀alkylene, —(CH₂CH₂O)_{zda}CH₂CH₂—, C₃₋₁₀cycloalkylene, C₆₋₁₀arylene C₁₋₁₀aheterocyclylene, or C₁₋₁₀aheteroarylene preferably A^{4a} is selected from null, C₁₋₁₀alkylene, or —(CH₂CH₂O)_{zda}CH₂CH₂—;
A^{4b} is selected from null, C₁₋₁₀alkylene, —(CH₂CH₂O)_{zdb}CH₂CH₂—,C₃₋₁₀cycloalkylene, C₆₋₁₀arylene, C₁₋₁₀aheterocyclylene, or C₁₋₁₀aheteroarylene; preferably A^{4b} is selected from null, C₁₋₁₀alkylene, or —(CH₂CH₂O)_{zdb}CH₂CH₂—;
A^{4c} is selected from null, C₁₋₁₀alkylene, —(CH₂CH₂O)_{zdc}CH₂CH₂—, C₃₋₁₀cycloalkylene, C₆₋₁₀arylene, C₁₋₁₀aheterocyclylene, or C₁₋₁₀aheteroarylene; preferably A^{4c} is selected from null, C₁₋₁₀alkylene, or —(CH₂CH₂O)_{zdc}CH₂CH₂—;
Z^{4a} is selected from null, —NH—, —O—, —S—, —NH(CO), —C(O)NH—, —O(CO)—, —(CO)O—, —NH(CO)O—, —OC(O)NH—, —NH(CO)NH,
Z^{4b} is selected from null, —NH—, —O—, —S—, —NH(O), —C(O)NH—, —O(CO)—, —(CO)O—, —NH(CO)O—, —OC(O)NH—, —NH(CO)NH,
Z^{4c} is selected from null, —NH—, —O—, —S—, —NH(CO), —C(O)NH—, —O(CO)—, —(CO)O—, —NH(CO)O—, —OC(O)NH—, —NH(CO)NH,

Z^{4d} is selected from null, —NH—, —O—, —S—, —NH(CO), —C(O)NH—, —O(CO)—, —(CO)—, —NH(CO)O—, —OC(O)NH—, —NH(CO)NH,

zda is 1, 2, 3, 4, 5, 6, 7, 8, 9, 10, 11, 12, 13, 14, 15, 16, 17, 18, 19, or 20;
zdb is 1, 2, 3, 4, 5, 6, 7, 8, 9, 10, 11, 12, 13, 14, 15, 16, 17, 18, 19, or 20; and
zdc is 1, 2, 3, 4, 5, 6, 7, 8, 9, 10, 11, 12, 13, 14, 15, 16, 17, 18, 19, or 20.

The X^{aa} group (N, NH, S, O, and C(O)) is derived from the side chain in the polypeptide; i.e., when X^{aa} is NH, the linker is connected via a lysine or arginine residue, when X^{aa} is S, the linker is connected via cysteine residue, when X^{aa} is N, the linker is connected via histidine or tryptophan residue, when X^{aa} is O, the linker is connected via a serine or tyrosine residue, and when X^{aa} is C(O), the linker is connected via an aspartic acid or glutamic acid residue. The skilled person understands that prior to coupling and/or functionalization, the polypeptide may be depicted:

[PP]—X^{aa}—H.

In certain embodiments Z¹ may have the formula:

—Z^{1a}—(CH₂)_{za1}—Z^{1b}—(CH₂)_{zb1}—Z^{1c}—(CH₂)_{ze1}—-Z^{1d}—,

wherein
za1 is 0, 1, 2, 3, 4, 5, 6, 7, 8, 9, or 10; preferably 0 or 2;
zb1 is 0, 1, 2, 3, 4, 5, 6, 7, 8, 9, or 10; preferably 0 or 2; and
zc1 is 0, 1, 2, 3, 4, 5, 6, 7, 8, 9, or 10; preferably 0 or 2.

In certain embodiments, Z² may have the formula:

—Z^{2a}—(CH₂)_{za2}—Z^{2b}—(CH₂)_{zb2}—Z^{2c}—(CH₂)_{ze2}—-Z^{2d}—,

wherein
za2 is 0, 1, 2, 3, 4, 5, 6, 7, 8, 9, or 10; preferably 0 or 2;
zb2 is 0, 1, 2, 3, 4, 5, 6, 7, 8, 9, or 10; preferably 0 or 2; and
zc2 is 0, 1, 2, 3, 4, 5, 6, 7, 8, 9, or 10; preferably 0 or 2.

In certain embodiments, Z³ may have the formula:

—Z^{3a}—(CH₂)_{za3}—Z^{3b}—(CH₂)_{zb3}—Z^{3c}—(CH₂)_{ze3}—-Z^{3d}—,

wherein
za3 is 0, 1, 2, 3, 4, 5, 6, 7, 8, 9, or 10; preferably 0 or 2;
zb3 is 0, 1, 2, 3, 4, 5, 6, 7, 8, 9, or 10; preferably 0 or 2; and
zc3 is 0, 1, 2, 3, 4, 5, 6, 7, 8, 9, or 10; preferably 0 or 2.

In certain embodiments, Z⁴ may have the formula:

—Z^{4a}—(CH₂)_{za4}—Z^{4b}—(CH₂)_{zb4}—Z^{4c}—(CH₂)_{ze4}—-Z^{4d}—,

wherein
za4 is 0, 1, 2, 3, 4, 5, 6, 7, 8, 9, or 10; preferably 0 or 2;
zb4 is 0, 1, 2, 3, 4, 5, 6, 7, 8, 9, or 10; preferably 0 or 2; and
zc4 is 0, 1, 2, 3, 4, 5, 6, 7, 8, 9, or 10; preferably 0 or 2.

In certain embodiments, the thermosensitive polymer is a PEG-PPG-PEG block copolymer, z is in both cases, X^{aa} is in both cases NH, and L¹ and L² are both —-C(O)O—, i.e., a compound having the formula:

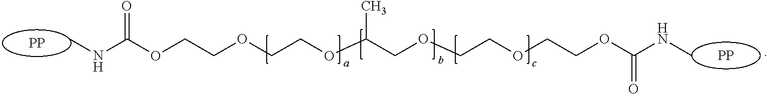

wherein a, b, and c are each independently selected from 1-1,000. In certain embodiments, b is selected from 25-100, from 25-75, from 50-100, from 25-50, from 50-75, from 40-60, or from 50-70, and a and c are each selected from 50-200, from 75-200, from 100-200, from 75-125, from 50-100, from 50-75, from 100-150, or from 90-110. Preferably, the polypeptide is gelatin, preferably a type A gelatin, and even more preferably having a Bloom number from 225 to 325.

The thermosensitive hydrogels disclosed herein may be combined with a number of different therapeutic agent, diagnostic and imaging agents, and other materials. Suitable materials include micelles, exosomes, liposome, polymersomes, inorganic self-assembled nanoparticles, metal nanoparticles, and combinations thereof. For example, the thermosensitive hydrogels may include metallic nanoparticles as antibiotics, for example gold nanoparticles, silver nanoparticles, copper nanoparticles, aluminum nanoparticles, zinc nanoparticles, and mixtures thereof. The metallic nanoparticles can be used for imaging, i.e., detectable upon exposure to irradiation. In one example, the imaging metallic nanoparticle is a Q-dots composed of cadmium, selenium, or indium. The metallic nanoparticles can be present in an amount, per gram of thermosensitive hydrogel, of at least 5 ng, at least 10 ng, at least 20 ng, at least 25 ng, at least 50 ng, at least 100 ng, at least 250 ng, at least 500 ng, at least 750 ng, at least 1,000 ng, at least 2,500 ng, at least 5,000 ng, at least 7,500 ng, or at least 10,000 ng. In some embodiments, the anti-microbial metallic nanoparticles are present in an amount, per gram of thermosensitive hydrogel, from 5-10,000 ng, from 5-5,000 nm, from 5-2,500 ng, from 5-1,000 ng, from 5-500 ng, from 5-250 ng, from 5-100 ng, from 5-50 ng, from 5-25 ng, from 100-2,500 ng, from 500-2,500 ng, from 1,000-2,500 ng, from 2,500-10,000 ng, or from 5,000-10,000 ng.

In some embodiments, the thermosensitive hydrogels may include one or more near-infrared (NIR) imaging agents. The near-infrared imaging agents may be used to diagnose various conditions, including solid tumors and other cancers. In one example, near-infrared (NIR) dyes include those showing light absorption from 700-2,000 nm. Exemplary near-infrared (NIR) organic dyes include squarylium, diimonium, and cyanine dyes. Exemplary dyes include:

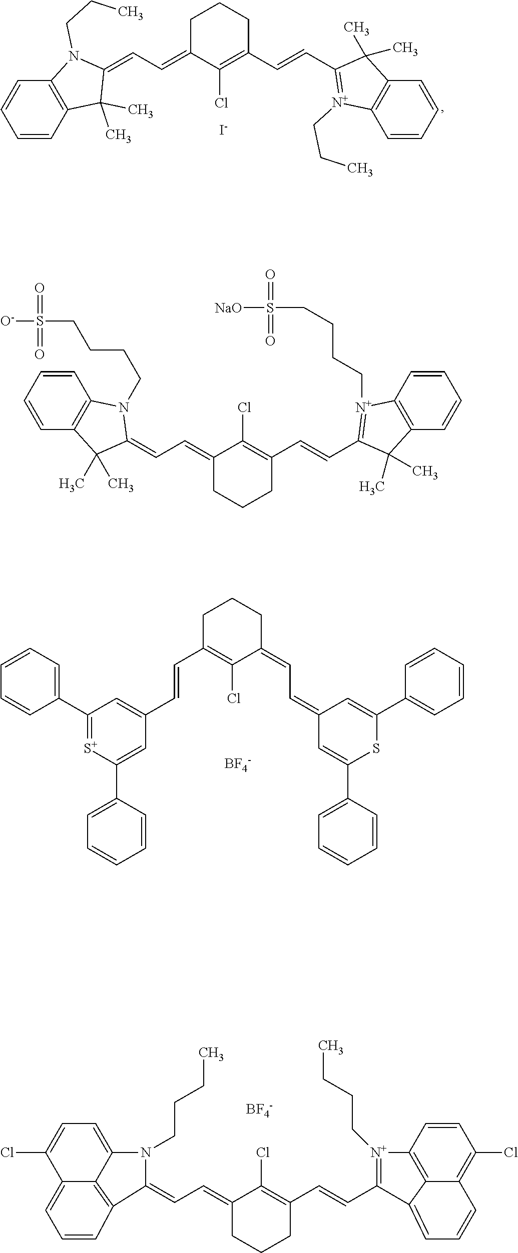

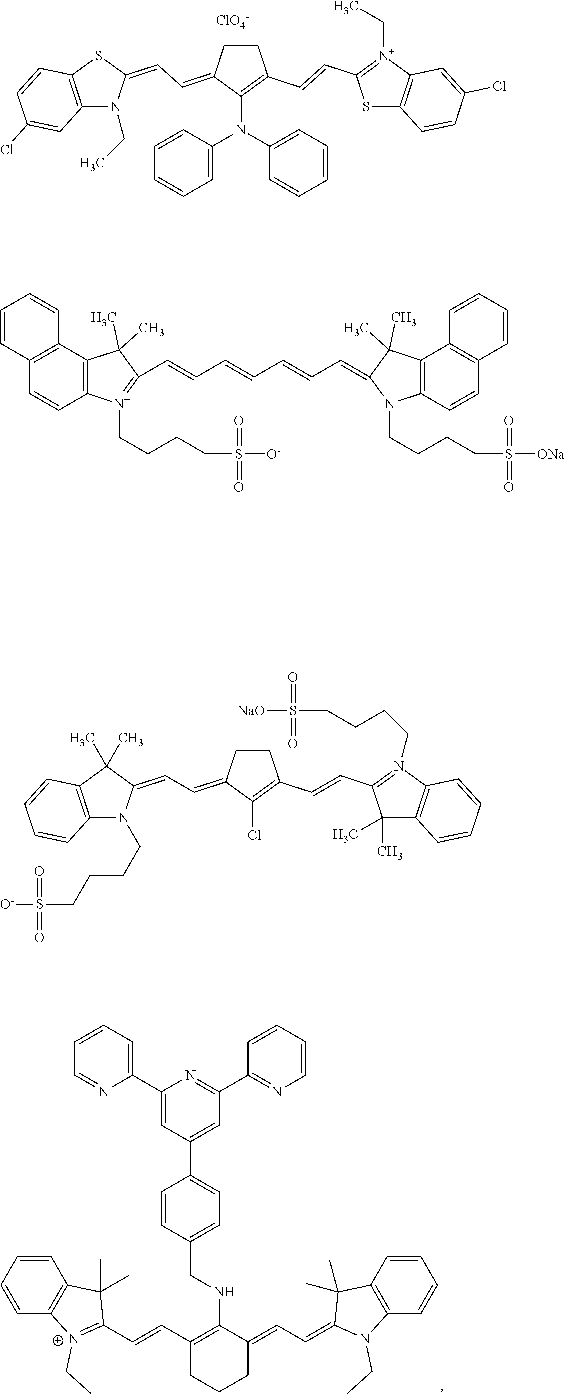

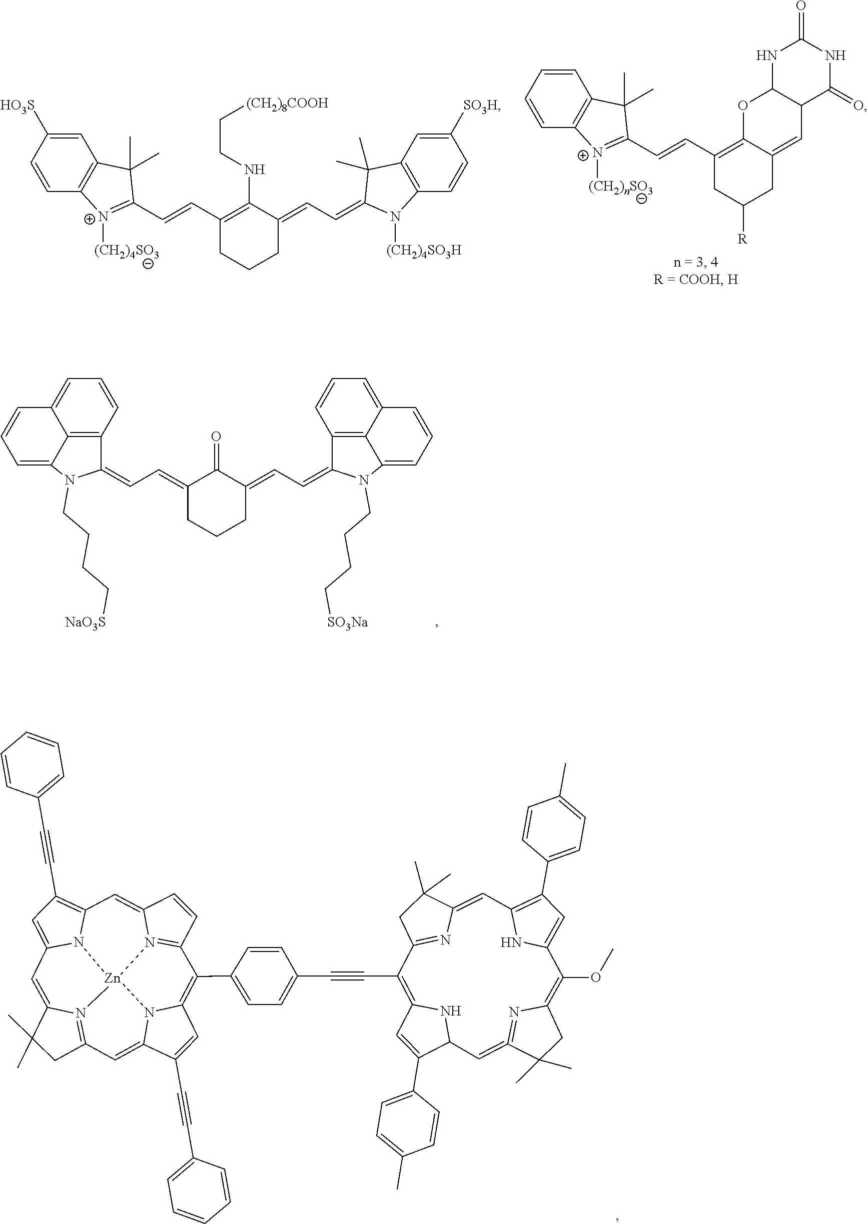

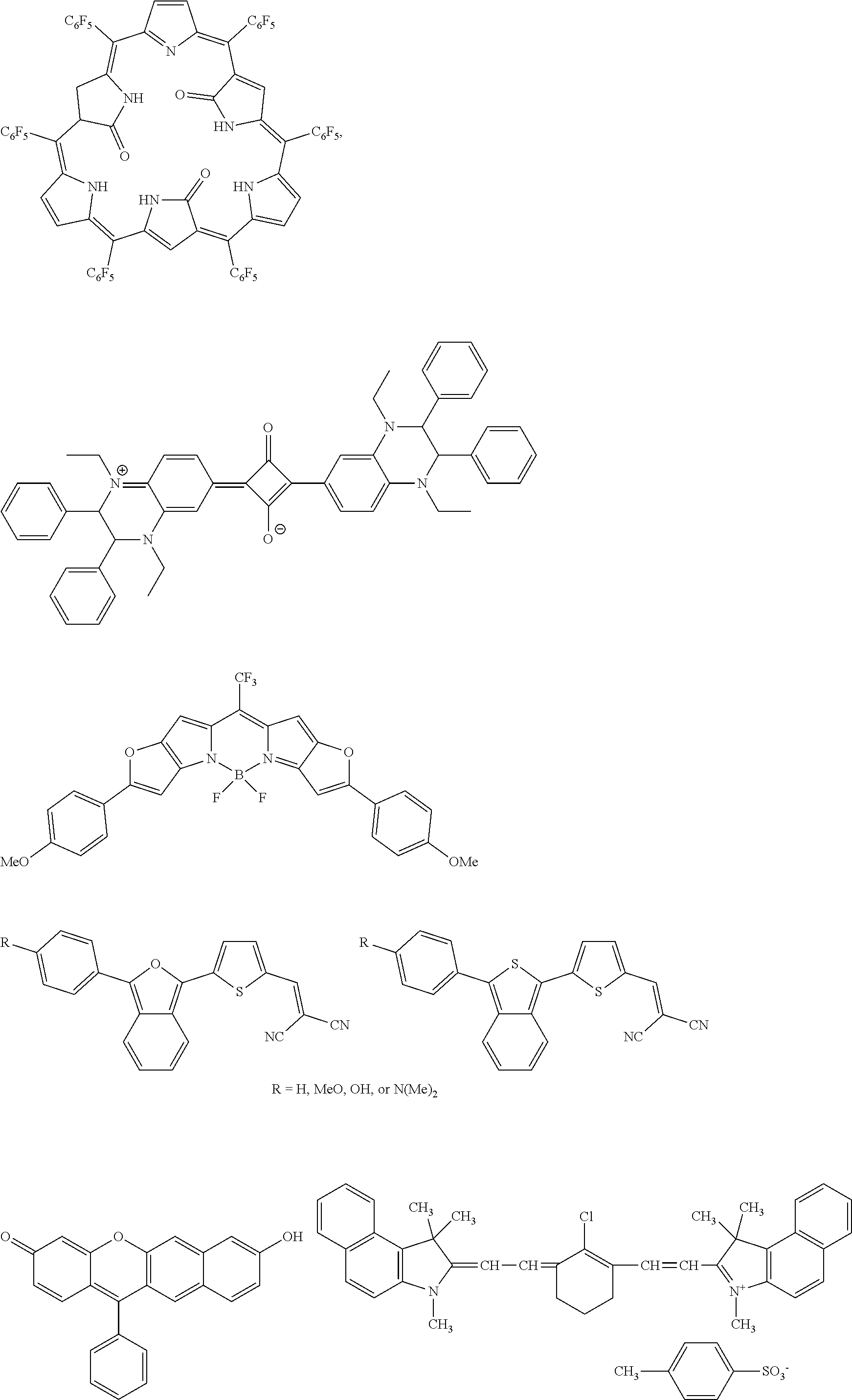

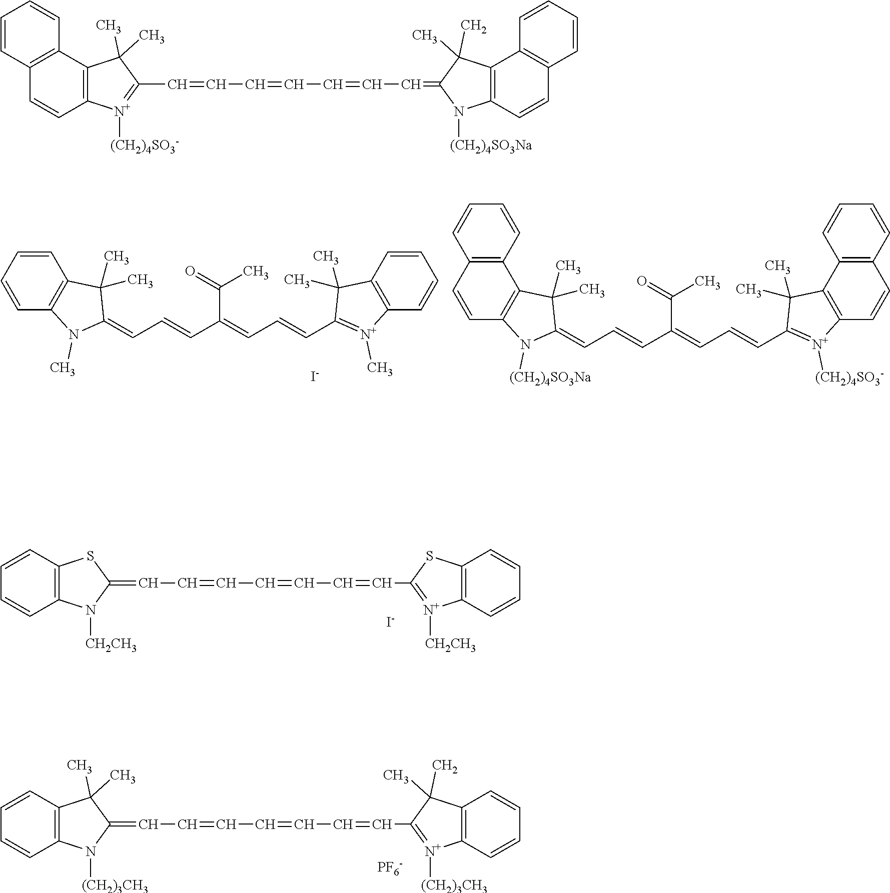

In certain embodiments, the thermosensitive hydrogels can include Q-dots, upconverting nanoparticles, and combinations thereof. Q-dots and upconverting nanoparticles absorb and emit radiation along wavelengths with useful clinical applications. In some embodiments, the Q-dots are nanoparticles having a semiconductor core (for example cadmium mixed with selenium or tellurium) and coated a different semiconductor (for example zinc sulfide). Upconverting nanoparticles are usually transition metals doped with one or more lanthanides or actinides.

The thermosensitive hydrogels disclosed herein may also be used for delivery of therapeutic agents. The hydrogels degrade into micelles with entrapped therapeutic agent, and these micelles are selectively taken up into the lymph system. As such, the thermosensitive hydrogels are especially useful in the context of drug delivery to the lymph.

In some embodiments, the thermosensitive hydrogels may include therapeutic agents such as NO-donors, immunomodulators, anti-cancer agents, and combinations thereof.

Exemplary immunomodulators include immune checkpoint blockade antibodies such as CTLA-4, PD-1, LAG-3, TIM-3, TIGIT, and combinations thereof. Exemplary immunomodulators may also include growth factors, cytokines, STING agonists, TLR agonists, and their combinations, but not limited.

STING agonists are compounds that agonize Stimulator of Interferon Genes, a transmembrane protein localized to the endoplasmic reticulum which functions as an adaptor protein in the WAS (cyclic GMP-AMP synthase)-STING pathway. Exemplary STING agonists include ADU-S100/MIW815, MK-1454, MK-2118, SB11285, GSK3745417, BMS-986301, BI-1387446, E7766, TAK-676, SNX281, SYN1981, JNJ-67544412, 3′3′-cyclic 3′3′-cAIMP, GSK432, GF3-002, TTI-10001, JNJ-′6196, CRD5500, CS-1018, CS-1020, CS-1010, MSA-1, ALG-031048, SR-8541A, SR-8314, ENPP1 inhibitors, and MV-616. In some embodiments, a thermosensitive hydrogel containing one of more STING agonists is administered to a patient in order to treat advanced/metastatic solid tumors or lymphomas, or unresectable, recurrent NHSCC.

Toll-like receptors (TLRs) are expressed primarily on monocytes, macrophages and DCs, which agonists promote inflammation. TLR agonists are under exploration for cancer immunotherapy as well as infectious diseases treatment, which include diacylated lipopeptides, triacylated lipoproteins, lipoteichoic acid, peptidoglycans, Zymosan, Poly I:C, Poly-ICLC (Hiltonol), LPS, HSPs, HMGB1, uric acid, snapin, tenascin C, Flagellin, CpG, CpG7909 (Promune), synthetic ssDNA (eg. DIMS0150, Kappaproct) Poly G10, Poly G3, fibronectin, PamCysPamSK4, Pam3CSK4, VSV, CFA, MALP2, Pam3Cys, FSL-1, Hib-OMPC, RC-529, MDF2beta, CFA, R848, Loxoribine, Imidazoquinolines, Imiquimod, Resiquimod, TMX-101, ssPolyU, 3M-012, Mycobacterium bovis (BCG), Mycobacterium indicus pranii (Cadi-05), Salmonella enterica flagellin (CBLB502, entolimod), Mycobacterium obuense (IMM-101), SD-101, VTX-2337 (motolimod), ISS1018, IMO-2055 (EMD1201081), Escherichia coli (OM-174, CRX-527), and Streptococcus pyogenes (Picibanil, OK-432). In some embodiments, a thermosensitive hydrogel containing one of more TLR agonists is administered to a patient in order to treat advanced/metastatic solid tumors or lymphomas, or unresectable, recurrent NHSCC.

Exemplary NO-donors include nitroglycerin, SIN-1, sodium nitroprusside, metal-nitrosyl complex including Mn—NO (Mn(PaPy₃)(NO)](ClO₄)) Cr(L)(ONO)₂⁺(L=cyclam=1,4,8,11-tetraazacyclotetradecane, CrONO, or L=mac=5,7-dimethyl-6-anthracenylcyclam, mac-CrONO), Roussin's Black Salt ([NH₄][Fe₄S₃(NO)₇]), and dinitrosyl iron complexes [Fe(μ-SEt)₂(NO)₄] (DNIC), S-nitrosothiols including S-nitrosoglutathione and S-nitroso-N-acetylpenicillamine (SNAP), diazen-1-ium-1,2-diolates (called as diazeniumdiolates or NONOates) including Spermine NONOate, Proline NONOate, MAHMA NONOate, DEA NONOate, DETA NONOate, DPTA NONOate, and PAPA NONOate, and O²-protected diazeniumdiolates including JS-K (O²-(2,4-Dinitrophenyl) 1[(4-Ethoxycarbonyl) piperazin-1-yl]diazen-1-ium-1,2-diolate), double JS-K (1,5-bis-{1-[(4-ethoxycarbonyl) piperazin-1-yl]diazen-1-ium-1,2-diol-2-ato}-2,4-dinitrobenzene), PABA/NO (O²-{2,4-dinitro-5-[4-(N-methylamino)benzoyloxy]phenyl} 1-(N,N-dimethylamino)diazen-1-ium-1,2-diolate), V-PYRRO/NO (O²-vinyl 1-(pyrrolidin-1-yl)diazen-1-ium-1,2-diolate), several V-IPA/NO (O²-Methyl 1-[4-(Ethoxycarbonyl)piperazin-1-yl]diazen-1-ium-1,2-diolate, O²-(2-Bromoethyl) 1-[4-(Ethoxycarbonyl)piperazin-1-yl]diazen-1-ium-1,2-diolate, O²-Acetoxymethyl 1-[4-(Ethyloxycarbonyl)piperazin-1-yl]diazen-1-ium-1,2-diolate, and O²-(3,4,6-Tri-O-acetyl-β-D-N-acetylglucosaminyl) 1- [4-(Ethoxycarbonyl)piperazin-1-yl]diazen-1-ium-1,2-diolate), O²-acetoxymethyl-protected diazeniumdiolate-based non-steroidal anti-inflammatory prodrugs (NONO-NSAIDS), P-NO (O²-(2,4-dinitrophenyl)-1-[N-methyl-(2,3-dihydroxypropyl) amino]-diazenium-1,2-diolate), and combinations thereof.

Exemplary anti-cancer agents include nucleoside analogues, antifolates, antimetabolites, topoisomerase I inhibitor, anthracyclines, podophyllotoxins, taxanes, vinca alkaloids, alkylating agents, platinum compounds, proteasome inhibitors, nitrogen mustards & oestrogen analogue, monoclonal antibodies, tyrosine kinase inhibitors, mTOR inhibitors, retinoids, immunomodulatory agents, histone deacetylase inhibitors, and combinations thereof.

In certain embodiments, the anti-cancer agent is selected from one or more of abiraterone acetate, methotrexate, paclitaxel albumin-stabilized nanoparticle, brentuximab vedotin, ado-trastuzumab emtansine, doxorubicin hydrochloride, afatinib dimaleate, everolimus, netupitant, palonosetron hydrochloride, imiquimod, aldesleukin, alectinib, alemtuzumab, melphalan hydrochloride, melphalan, pemetrexed disodium, chlorambucil, aminolevulinic acid, anastrozole, aprepitant, pamidronate disodium, exemestane, nelarabine, arsenic trioxide, ofatumumab, asparaginase erwinia chrysanthemi, atezolizumab, bevacizumab, axitinib, azacitidine, carmustine, belinostat, bendamustine hydrochloride, bevacizumab, bexarotene, tositumomab, bicalutamide, bleomycin, blinatumomab, blinatumomab, bortezomib, bosutinib, busulfan, cabazitaxel, cabozantinib, alemtuzumab, irinotecan hydrochloride, capecitabine, fluorouracil, carboplatin, carfilzomib, bicalutamide, lomustine, ceritinib, daunorubicin hydrochloride, cetuximab, chlorambucil, cyclophosphamide, clofarabine, cobimetinib, dactinomycin, cobimetinib, crizotinib, ifosfamide, ramucirumab, cytarabine, dabrafenib, dacarbazine, decitabine, daratumumab, dasatinib, daunorubicin hydrochloride, decitabine, efibrotide sodium, defibrotide sodium, degarelix, denileukin diftitox, denosumab, dexamethasone, dexrazoxane hydrochloride, dinutuximab, docetaxel, doxorubicin hydrochloride, dacarbazine, rasburicase, epirubicin hydrochloride, elotuzumab, oxaliplatin, eltrombopag olamine, aprepitant, elotuzumab, enzalutamide, epirubicin hydrochloride, cetuximab, eribulin mesylate, vismodegib, erlotinib hydrochloride, etoposide, raloxifene hydrochloride, melphalan hydrochloride, toremifene, panobinostat, fulvestrant, letrozole, filgrastim, fludarabine phosphate, flutamide, methotrexate, pralatrexate, recombinant hpv quadrivalent vaccine, recombinant hpv nonavalent vaccine, obinutuzumab, gefitinib, gemcitabine hydrochloride, gemtuzumab ozogamicin, afatinib dimaleate, imatinib mesylate, glucarpidase, goserelin acetate, eribulin mesylate, trastuzumab, topotecan hydrochloride, palbociclib, ibritumomab tiuxetan, ibrutinib, ponatinib hydrochloride, idarubicin hydrochloride, idelalisib, imiquimod, axitinib, recombinant interferon alfa-2b, tositumomab, ipilimumab, gefitinib, romidepsin, ixabepilone, ixazomib citrate, ruxolitinib phosphate, cabazitaxel, ado-trastuzumab emtansine, palifermin, pembrolizumab, lanreotide acetate, lapatinib ditosylate, lenalidomide lenvatinib mesylate, leuprolide acetate, olaparib, vincristine sulfate, procarbazine hydrochloride, mechlorethamine hydrochloride, megestrol acetate, trametinib, mercaptopurine, temozolomide, mitoxantrone hydrochloride, plerixafor, busulfan, azacitidine, gemtuzumab ozogamicin, vinorelbine tartrate, necitumumab, nelarabine, sorafenib tosylate, nilotinib, ixazomib citrate, nivolumab, romiplostim, obinutuzumab, ofatumumab, olaparib, omacetaxine mepesuccinate, pegaspargase, ondansetron hydrochloride, osimertinib, panitumumab, panobinostat, peginterferon alfa-2b, pembrolizumab, pertuzumab, plerixafor, pomalidomide, ponatinib hydrochloride, necitumumab, pralatrexate, procarbazine hydrochloride, aldesleukin, denosumab, ramucirumab, rasburicase, regorafenib, lenalidomide, rituximab, rolapitant hydrochloride, romidepsin, ruxolitinib phosphate, siltuximab, dasatinib, sunitinib malate, thalidomide, dabrafenib, osimertinib, talimogene, atezolizumab, temsirolimus, thalidomide, dexrazoxane hydrochloride, trabectedin, trametinib, trastuzumab, lapatinib ditosylate, dinutuximab, vandetanib, rolapitant hydrochloride, bortezomib, venetoclax, crizotinib, enzalutamide, ipilimumab, trabectedin, ziv-aflibercept, idelalisib, ceritinib, and pharmaceutically acceptable salts thereof.

In some embodiments, the therapeutic agent is a monoclonal antibody. The monoclonal antibody may be a cancer therapeutic such as described above including immune checkpoint inhibitors, or may have a different intended use. For example, the monoclonal antibody may operate blocking and/or depleting T cells, B cells, or both. The monoclonal antibody may inhibit of the interaction between T cells and antigen-presenting cells, block T- and B-cell recruitment, block T-cell differentiation or activation, and/or block pro-inflammatory cytokines. Exemplary monoclonal antibodies exhibiting these features include adalimumab, alemtuzumab, belimumab, benralizumab, brodalumab, canakinumab, certolizumab pegol, golimumab, guselkumab, infliximab, itolizumab, ixekizumab, mepolizumab, natalizumab, ocrelizumab, omalizumab, reslizumab, risankizumab, rituximab, ruplizumab, sarilumab, secukinumab, tildrakizumab, tocilizumab, ustekinumab, and vedolizumab.

In some embodiments, the monoclonal antibody can be intended for the treatment of infectious diseases, such those caused by coronavirus, cytomegalovirus, hepatitis A and hepatitis B viruses. Exemplary antibodies include bezlotoxumab, ibalizumab, oblitoxaximab, palivizumab, raxibacumab, and Rmab.

In other embodiments, the monoclonal antibody can be abciximab (antiplatelet therapy), erenumab, fremanezumab, and galcanezumab (each of which can be used for migraines).

In some embodiments, the monoclonal antibody can be a PCSK9 inhibitor, such as evolocumab, bococizumab, alirocumab, 1D05-IgG2 (Merck), RG-7652 and LY3015014.

In some embodiments the monoclonal antibody can be indicated for the treatment of hematologic malignancies, solid tumors, autoimmune disorders (including disorders with an immune component), hypercholesterolemia, asthma, osteoporosis, neurological disorders, allograft or transplant rejection, or infectious organisms.

In particular embodiments, the monoclonal antibody can be used for one or more of the following indications: soft tissue sarcoma, prevention of Clostridium difficile infection recurrence, prevention of inhalational anthrax, lymphoblastic leukemia, atopic dermatitis, rheumatoid arthritis, Merkel cell carcinoma, multiple sclerosis, hemophilia A, asthma, myeloid leukemia, bladder cancer, X-linked hypophosphatemia, hereditary angioedema attacks, mycosis fungoides, Sézary syndrome, migraine prevention, plaque psoriasis, cutaneous squamous cell carcinoma, primary hemophagocytic lymphohistiocytosis, migraine prevention, HIV infection, hairy cell leukemia, paroxysmal nocturnal hemoglobinuria, acquired thrombotic thrombocytopenic purpura, osteoporosis, plaque psoriasis, diffuse large B-cell lymphoma, macular degeneration, and sickle cell disease. Thus, disclosed herein is a method of treating one or more of the indication listed above, by providing to a subject in need thereof, a thermosensitive hydrogel loaded with an effective amount of an appropriate monoclonal antibody.

The monoclonal antibodies disclosed herein can be conjugated to a drug or other therapeutic agent, while in some embodiments the monoclonal antibody is not conjugated to a further therapeutic agent. Exemplary antibody drug conjugates include moxetumomab pasudotox, polatuzumab vedotin, and brentuximab vedotin Such conjugates are design, for example an anticancer drug.

In some embodiments, the therapeutic hydrogels may include multiple drugs including combinations of immune checkpoint inhibitors, NO-donors, monoclonal antibodies, and/or chemotherapeutic drugs. Single immune checkpoint blockade antibody, single NO-donor, single chemotherapeutic drug, combined monoclonal antibodies, combined immune checkpoint blockade antibodies, combined NO-donors, combined chemotherapeutic drugs, combined immune checkpoint blockade antibodies and NO-donors, combined immune checkpoint blockade antibodies and chemotherapeutic drugs, combined chemotherapeutic drugs and NO-donors, or combined immune checkpoint blockade antibodies, NO-donors, and chemotherapeutic drugs can included in the therapeutic hydrogels with their free forms or with other DDSs chemically loading immune checkpoint blockade antibodies, NO-donors, and/or chemotherapeutic drugs.

In certain embodiments, the active agent can be one or more growth factors, e.g., protein growth factors. A growth factor (either naturally or non-naturally occurring) stimulates cell proliferation, would healing, and/or cellular differentiation. Exemplary growth factors that can be included in the thermosensitive hydrogels include bone morphogenic protein, adrenomedullin, autocrine motility factor, ciliary neurotropic factor, leukemia inhibitory factor, interleukin-6, epidermal growth factor, ephrins (A1, A2, A3, A4, A5, B1, B2, B3, and combinations thereof), insulin, neurturin epoetin, darbepoetin alfa, luspatercept, granulocyte colony stimulating factor (G-CSF, filgrastim, or Neupogen), granulocyte macrophage-colony stimulating factor (GM-CSF, sargramostim, or Leukine), pegfilgrastim, romiplostim, eltrombopag, oprelvekin, ancestim, Aranesp, benegrastim, eflapegrastim, Epogen, Eprex, erythropoietin, filgrastim, filgrastim-aafi, filgrastim-sndz, Fulphila, Granix, interleukin 11, Mercera, Neumega, Nivestym, Nplate, Nyvepria, oprelevkin, Procrit, Promacta, Retacrit, Rolontis, Rysneuta, sargramostim, Stemgen, Udenyca, Zarxio, Ziextenzo, and combinations thereof.

Particularly preferred combinations include aPD-1 and vemurafenib.

Particularly preferred combinations include aCTLA-4 and GSNO.

The therapeutic or diagnostic agent may be conjugated to the thermosensitive hydrogel, for instance using similar conjugation strategies described above for conjugation of the thermosensitive polymer to the polypeptide. In some embodiments, a first terminus of the linear thermosensitive polymer may be covalently conjugated to the therapeutic or diagnostic agent, and the remaining terminus conjugated to the polypeptide. Depending on the desired dosage level of the therapeutics or diagnostics, a portion of the thermosensitive polymers may be functionalized this way, and then combined second portion of thermosensitive polymers not conjugated to a therapeutic or diagnostic agent, followed by conjugation to the polypeptide. In such embodiments, thermosensitive polymers having different functional groups at each terminus (e.g., polyesters) may be advantageously used. For embodiments in which the thermosensitive polymer is a branched polymer, one or more termini may be covalently conjugated to the therapeutic or diagnostic agent. For example, a three-armed thermosensitive polymer may be conjugated to a therapeutic or diagnostic agent at one or two termini, a four-armed thermosensitive polymer may be conjugated at one, two, or three termini, a five-armed thermosensitive polymer may be conjugated at one, two, three, or four termini. The therapeutic or diagnostic agent-thermosensitive polymer conjugate may be from 0.01-5%, from 0.01-2.5%, from 0.01-1.0%, from 0.01-0.5%, from 0.01-0.1%, from 0.1-5%, from 0.5-2.5%, or 0.5-1.5% by weight, relative to the total weight of the thermosensitive polymer. In some embodiments, the amount of thermo sensitive polymer conjugated to a therapeutic or diagnostic agent is present in a non-zero amount, but no more than 50%, no more than 40%, no more than 30%, no more than 20%, no more than 10%, no more than 5%, no more than 2.5%, no more than 1%, or no more than 0.5% by weight, relative to the total weight of the thermosensitive polymer.

In certain embodiments, the therapeutic or diagnostic agent may be present in the hydrogel (whether chemical (i.e., covalently) conjugated or simply dispersed) in an amount from 0.01-10%, from 1-10%, from 2-10%, from 5-10%, from 2-5%, from 2-7%, from 3-6%, from 0.01-5%, from 0.01-2.5%, from 0.01-1.0%, from 0.01-0.5%, from 0.01-0.1%, from 0.1-5%, from 0.5-2.5%, or 0.5-1.5% by weight, relative to the total weight of the thermosensitive hydrogel. In some embodiments, the therapeutic or diagnostic agent is present in a non-zero amount, but no more than 10%, no more than 5%, no more than 2.5%, no more than 1%, no more than 0.5%, no more than 0.25%, no more than 0.1%, no more than 0.05%, or no more than 0.01% by weight, relative to the total weight of the thermosensitive hydrogel.

In other embodiments, the therapeutic or diagnostic agent may be loaded into the thermosensitive hydrogel by passive diffusion, for instance, by hydrating a dried hydrogel in an aqueous solution containing the therapeutic or diagnostic agent, or by equilibrating a hydrated hydrogel in a solution of therapeutic or diagnostic agent such that the concentration of the therapeutic or diagnostic agent is the same in the solution and within the hydrogel. Such solutions may contain further excipients as described herein.

In addition to the therapeutic or diagnostic agent, the hydrating and equilibrating solutions may further include a solvent or dispersion medium containing, for example, water, ethanol, one or more polyols (e.g., glycerol, propylene glycol, 1,4-butanediol, and liquid polyethylene glycols), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, glycerin, sugars like mannitol or sorbitol, sodium chloride as well as dispersants, emulsifiers, pH modifying agents, and the like.

Suitable surfactants include anionic, cationic, amphoteric or nonionic surface-active agents. Suitable anionic surfactants include, but are not limited to, those containing carboxylate, sulfonate and sulfate ions. Suitable anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Suitable cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Suitable nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

The thermosensitive hydrogels can include one or more preservatives to prevent the growth of microorganisms. Suitable preservatives include, but are not limited to, polyhexamethylenebiguanide (PHMB), benzalkonium chloride (BAK), stabilized oxychloro complexes (otherwise known as Purite®), phenol, phenylmercuric acetate, chlorobutanol, sorbic acid, chlorhexidine, chlorobutanol, benzyl alcohol, parabens, thimerosal, and mixtures thereof.

The thermosensitive hydrogel can be buffered to a pH of 3-8, from 4-8, from 5-8, from 6-8, from 7-8, from 3-7, from 3-6, from 3-5, from 3-4, from 4-7, from 4-6, from 4-5, from 5-7, or from 6-7, or from 5-6. Suitable buffers include, but are not limited to, phosphate buffers, acetate buffers, borate buffers, and citrate buffers.

The thermosensitive hydrogels can include one or more water-soluble polymers, including, but are not limited to, polyvinylpyrrolidone, dextran, carboxymethylcellulose, albumin, chitosan, gelatin, hyaluronic acid, and polyethylene glycol.

The thermosensitive hydrogels, either with or without one or more therapeutic or diagnostic agents and/or excipients, may be provided in the form or lyophilized powder. In some embodiments the therapeutic or diagnostic agent is preloaded into the hydrogel, and the composition then hydrolyzed. In other embodiments the lyophilized thermosensitive hydrogel is hydrated with a solution containing the therapeutic or diagnostic agent.

Depending on the clinical application, the composition of the thermosensitive hydrogel containing one or more therapeutic or diagnostic agents may be delivered via various administration routes. For example, the composition may be injected intradermally (i.d.) ipsilaterally (i.l.) to a tumor. Intradermally (i.d.) administration, including to the tissue contralateral (c.l) to the tumor, intratumorally (i.t.), intravenously (i.v.), and intraperitoneally (i.p.) administrations are also available with thermosensitive hydrogels and micelles. In other embodiments, the compositions may be applied topically, for instance to the surface of the eye, to a wound and/or lesion, or to other affected tissue.

### EXAMPLES

The following examples are for the purpose of illustration of the invention only and are not intended to limit the scope of the present invention in any manner whatsoever. Unless indicated otherwise, parts are parts by weight, temperature is in ° C. or is at ambient temperature, and pressure is at or near atmospheric.

### Example 1: Synthesis and Characterization of a Thermosensitive Hydrogel

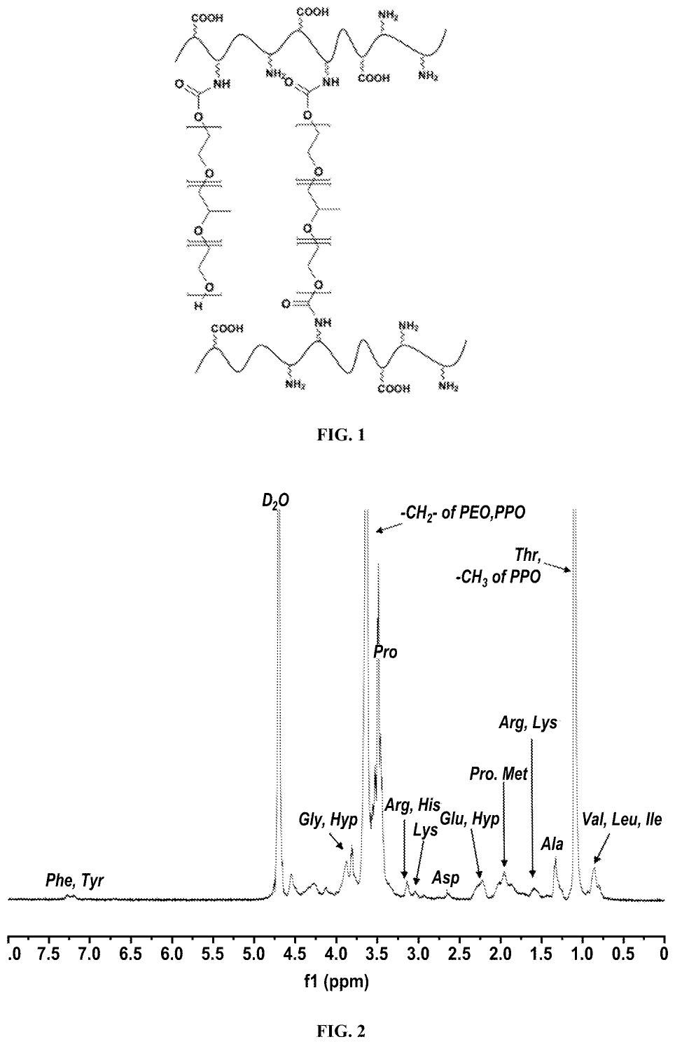

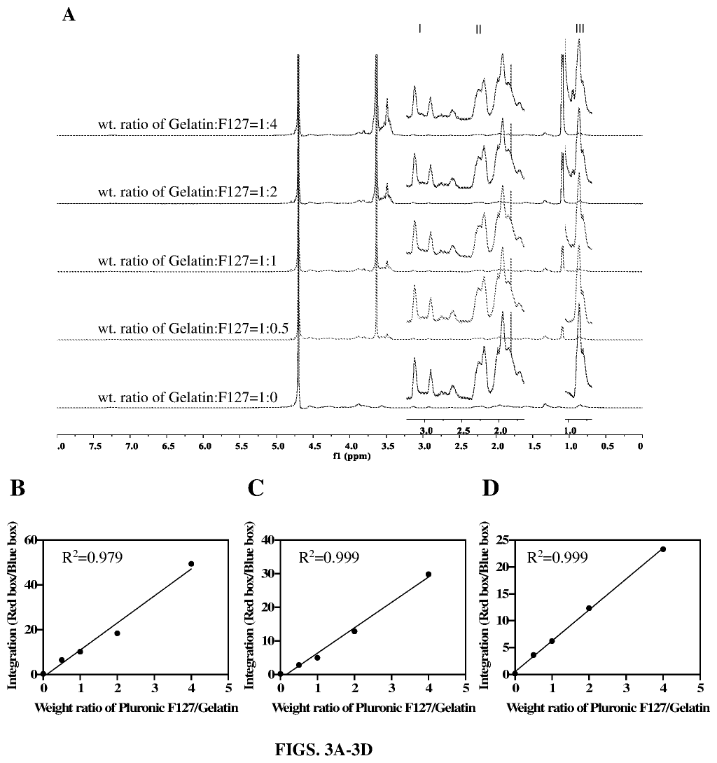

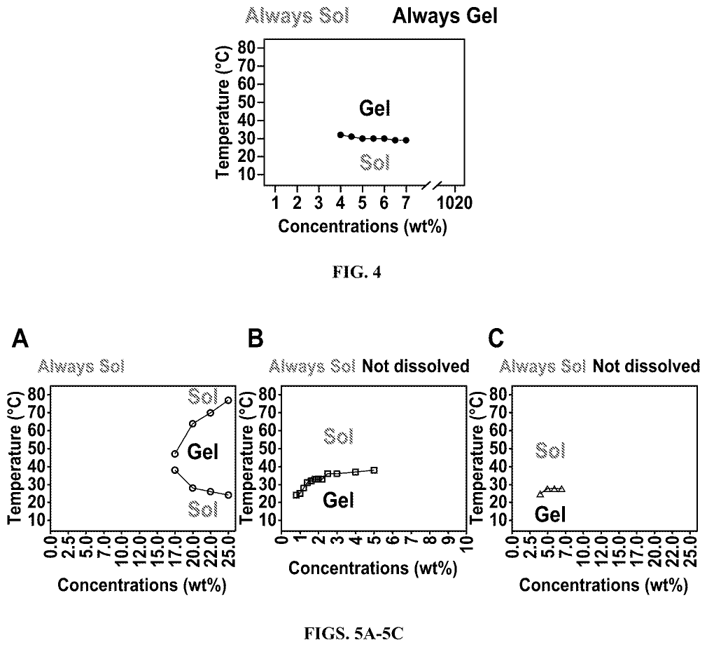

20 g of Pluronic® F127 (Sigma Aldrich, F127) in 50 mL dichloromethane (Sigma Aldrich, DCM) was reacted with 3.2 g of 4-nitrophenyl chloroformate (Sigma Aldrich, p-NPC) in 50 mL DCM with vigorous stirring for overnight, followed by precipitation in 2750 mL cold diethyl ether (Sigma Aldrich) and vacuum filter. The resultant p-NPC activated F127 in 150 mL of 33.3% ethanol was vigorously mixed with 10 g of gelatin type A (Sigma Aldrich, 300 g bloom) in 1,000 mL deionized water containing 15 mL triethylamine (Sigma Aldrich) for overnight. 1.5 day of Dialysis against deinoized water (Spectrum Industries, MWCO 100 KDa) and 3 days of freezing drying resulted in F127-g-Gelatin. ¹H nuclear magnetic resonance spectroscopy (¹H NMR) with Bruker Advance 400 MHz FT-NMR confirmed the successful synthesis and chemical compositions of F127-g-Gelatin (FIGS. 2 and 3); 35.1 wt. % Gelatin and 64.9±1.97 wt. % F127.

The thermosensitivity of F127-g-Gelatin was investigated by vial tilting method. The F127-g-Gelatin formed thermosensitive hydrogels at very low concentrations (4.0-7.0 wt. %) end though gelatin along behaves upper critical solution temperature (UCST) that transit from gel to sol at temperature higher than critical temperature and 37° C.(FIG. 5). However, the simple admixture of F127 and gelatin did not show the enhanced thermosensitive behavior (FIG. 5). Data are presented in Tables 1 and 2.

TABLE 1
Summary of sol-gel and gel-sol transition of
F127 ® and F127-g-Gelatin.

| | Sol→Gel T (° C.)/Gel→Sol T (° C.) | | F127 wt % in |
|---|---|---|---|
| wt % | F127 | F127-g-Gelatin | F127-g-Gelatin |
| 0.5 | Always sol | Always sol | 0.32 |
| 1 | (4-85° C.) | (4-85° C.) | 0.65 |
| 1.5 | | | 0.97 |
| 2 | | | 1.29 |
| 2.5 | | | 1.62 |
| 3 | | | 1.94 |
| 3.5 | | | 2.26 |
| 4 | | 32° C./Not Available | 2.59 |
| | | (N.A.) | |
| 4.5 | | 31° C./N.A. | 2.91 |
| 5 | | 30° C./N.A. | 3.24 |
| 5.5 | | 30° C./N.A | 3.56 |
| 6 | | 30° C./N.A | 3.88 |
| 6.5 | | 29° C./N.A | 4.21 |
| 7 | | 29° C./N.A | 4.53 |
| 7.5 | | Always gel | 4.85 |
| 10 | | (4-85° C.) | 6.47 |
| 15 | | | 9.71 |
| 17.5 | 38° C./47° C. | | 11.32 |
| 20 | 28° C./64° C. | | 12.94 |
| 22.5 | 26° C./70° C. | | 14.56 |
| 25 | 22° C./77° C. | | 16.18 |

TABLE 2
Summary of sol-gel and gel-sol transition of Gelatin and the mixture
of Gelatin and F127 (Gelatin:F127 = 35.1 wt. %:64.9 wt. %).

| | | | F127/Gelatin mixture |
|---|---|---|---|
| | Gelatin | | (64.9%:35.1%) |
| | Gel→Sol T (° C.)/ | | Gel→Sol T (° C.)/ |
| wt % | Sol→Gel T (° C.) | wt % | Sol→Gel T (° C.) |
| 0.2 | Always sol | 1 | Always sol |
| 0.4 | (4-85° C.) | 2 | (4-85° C.) |
| 0.6 | | 4 | 25° C./N.A. |
| 0.8 | 24° C./N.A. | 5 | 28° C./N.A. |
| 1 | 25° C./N.A. | 6 | 28° C./N.A. |
| 1.2 | 28° C./N.A. | 7 | 28° C./N.A. |
| 1.4 | 31° C./N.A. | 8 | Not dissolved |
| 1.6 | 32° C./N.A. | 10 | |
| 1.8 | 33° C./N.A. | 12.5 | |
| 2 | 33° C./N.A. | 15 | |
| 2.2 | 33° C./N.A. | >17.5 | |
| 2.5 | 36° C./N.A. | | |
| 3 | 36° C./N.A. | | |
| 4 | 37° C./N.A. | | |
| 5 | 38° C./N.A. | | |
| >5.5 | Not dissolved in 25° C. | | |

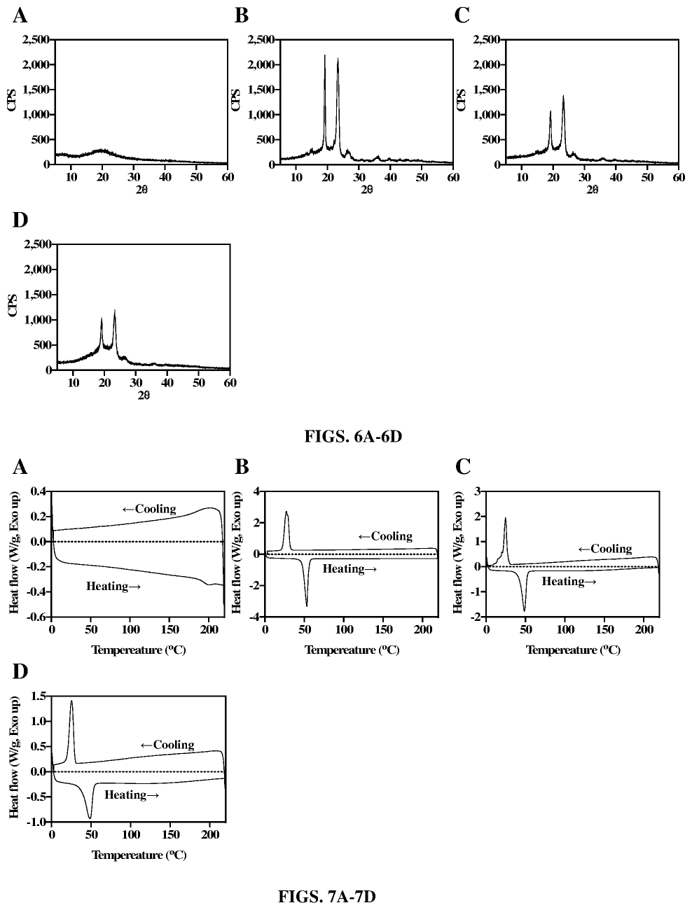

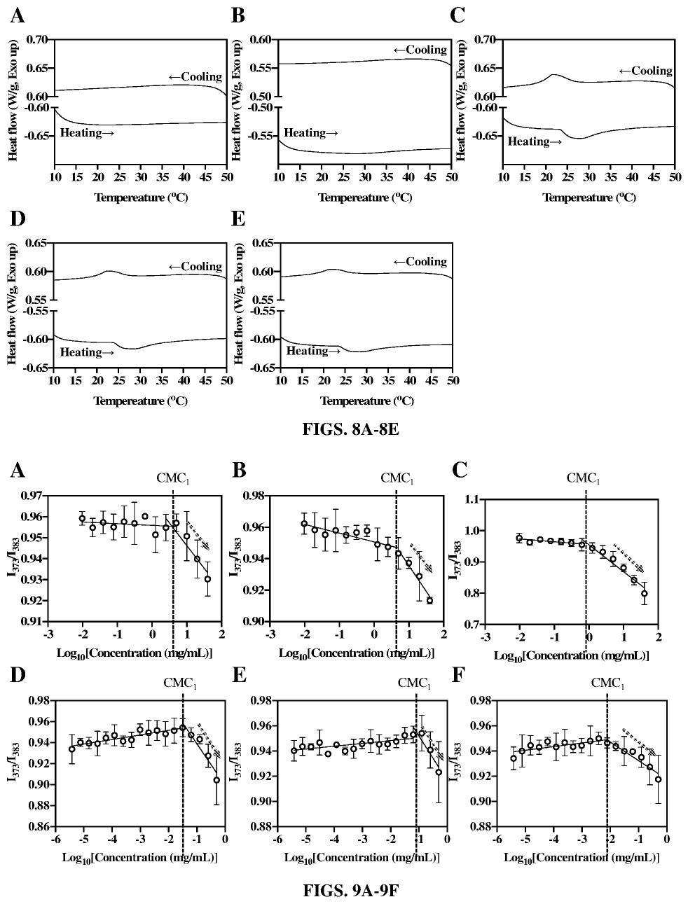

Powder crystallinity of lyophilized 4 wt. % hydrogels was investigated by using differential scanning calorimeter (TA Instruments Q200, DSC) and X-ray diffraction (Malvern PANalytical Empyrean, XRD). Solution crystallinity of polymers were evaluated in 2 wt. % polymer solutions with DSC. In XRD, crystalline peak for triple-helix (°2θ=8.4) was reduced and peak for amorphous phase (°2θ=21.1) was increased in F127-g-gelatin, compared to gelatin (FIG. 6). In addition, F127-g-gelatin showed the reduced crystalline peak for F127 (°2θ=19.2 and 23.4), compared to F127 (FIG. 6). Furthermore, there were no additional crystalline peaks for F127-g-Gelatin in DSC, compared to gelatin and F127 (FIGS. 7 and 8).

Ratiometric quantification of emitted fluorescence (373 nm and 383 nm) of pyrenes at excitation wavelength of 336 nm allowed the determination of critical micellar concentrations (CMC). Accordingly, 50 μL, of different concentrations of polymers was incubated with 50 μL of 1.2 μM pyrene for 1 day. In F127-g-Gelatin, CMC was significantly decreased with the increase of temperature-dependency in CMC, compared to F127 and the mixture of F127 and gelatin (FIG. 9, Table 3). Therefore, it can be concluded that the improved thermosensitivity of F127-g-Gelatin is attributed to the enhanced amorphous hydrophobic interactions.

TABLE 3
CMC of F127, mixture of Gelatin and F127 (Gelatin:F127 = 35.1
wt. %:64.9 wt. %) and F127-g-Gelatin at RT and 37° C.

| | | F127 | Mixture | F127-g-Gelatin | Fold (F127/F127-g-Gelatin) |
|---|---|---|---|---|---|
| CMC₁ | 25° C. | 3.55 ± 1.00 | 4.45 ± 0.54 | 0.98 ± 0.24 | 3.62 |
| (mg mL⁻¹) | 37° C. | 0.041 ± 0.013 | 0.11 ± 0.011 | 0.0076 ± 0.0026 | 5.39 |

| | | | |
|---|---|---|---|
| Fold (25° C./37° C.) | 87.18 | 40.9 | 129.06 |

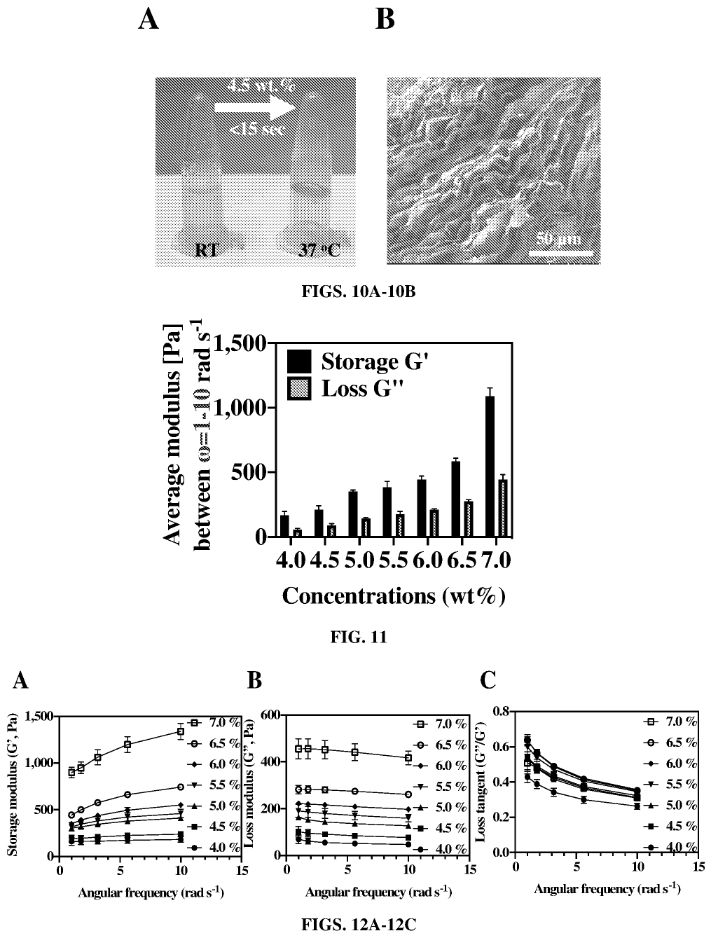

The lyophilized 4.5 wt. % hydrogel was imaged with scanning electron microscopy (SEM) equipped with Hitachi SU-8230 at accelerating voltage 1 kV and 10 μA emission current, which exhibited sheet-like microstructures (FIG. 10).

The rheology of F127-g-Gelatin hydrogels was evaluated with dynamic oscillatory strain and frequency sweeps on a Discovery HR-2 rheometer (TA Instruments) with an 8 mm diameter, flat geometry at an angular frequency (ω) of 1-10 rad s⁻¹ (Plate SST 8 mm Smart-Swap, TA Instruments), which exhibited concentration-dependent rheology at 37° C. (FIG. 11,12).

### Example #2—In Vitro Residence Stability and Release of GSNO and aCTLA-4 from Hydrogel

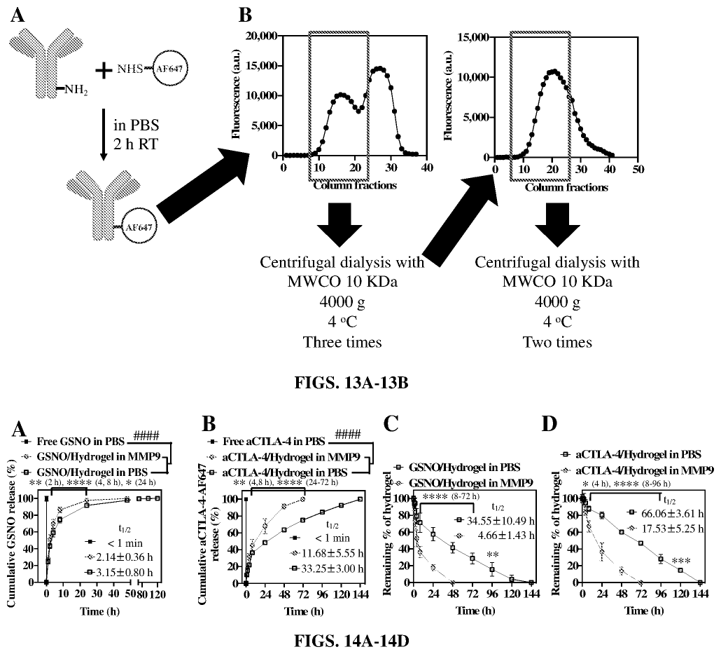

In order to prepare Alexa Fluor™ 647 labeled aCTLA-4 (aCTLA-4-AF647) to be used for drug release test (FIG. 13), 5.4 mg of aCTLA-4 (BioXCell, clone: 9H10) in 600 μL PBS was mixed with 35 μL of 10 mM Alexa Fluor™ 647 NHS Ester (AF647-NHS) (Invitrogen™) in DMSO under mild stirring at room temperature for 2 h. CL-6B Sepharose® column (GE Healthcare) and spin filter with Amicon® Ultra centrifugal filter (Millipore, MWCO 30 kDa) at 4000 g and 4° C. for 20 min allowed to purify aCTLA-4-AF647.

In order to investigate the in vitro drug release behaviors of F127-g-gelatin hydrogels, GSNO (Sigma Aldrich) (final GSNO concentrations equivalent to 0.45 mg mL⁻¹) or aCTLA-4-AF647 (final aCTLA-4 concentrations equivalent to 0.542 mg mL⁻¹) were loaded into 300 μL of F127-g-gelatin 4.5 wt. % hydrogel in 1.5 mL e-tube in 37° C. water incubator and then incubated with additional 300 μL of PBS or 2.5 U mL⁻¹ MMP-9 (Gibco™, collagenase IV). Supernatants was sampled at predetermined time intervals. Fresh 300 μL of PBS or 2.5 U mL⁻¹ MMP-9 was added to the e-tube after recording the remaining mass of the hydrogels to investigate the residence stability of the hydrogels. Typical Griess/Saville assay on the supernatant samples yielded at predetermined time intervals was employed to evaluate GSNO release. aCTLA-4 release was quantified by recording AF647 fluorescence (650 nm excitation, 670 nm emission) of supernatant samples yielded at predetermined time intervals with Synergy H4 microplate. GSNO and aCTLA-4-AF647 in 4.5 wt. % F127-g-gelatin hydrogels were released in a sustained manner (FIG. 14), but their release could be accelerated by enzymatic degradation with MMP-9 (FIG. 14) normally overexpressed by melanoma. Interestingly, F127-g-Gelatin hydrogels containing aCTLA-4-AF647 showed longer residence time and release half-life in vitro than F127-g-Gelatin hydrogels containing GSNO (FIG. 14A-D). These results imply the involvement of aCTLA-4 in the process of F127-g-gelatin hydrogel formation.

### Example #3—Confirmation and Characterizations of In-Situ F127-g-Gelain Micelles Containing aCTLA-4

In order to investigate whether aCTLA-4 is associated or interacts with F127-g-gelatin, size and zeta potential of in situ micelles released from F127-g-gelatin hydrogels with or without aCTLA-4 were assessed by dynamic light scattering (DLS) and Zetasizer Nano ZS (Malvern Instruments). The final concentrations of F127-g-gelatin and aCTLA-4 after totally released from 4.5 wt. % F127-g-gelatin hydrogel were equivalent to 0.9 wt. % and 0.542 mg mL⁻¹, respectively. Indeed, the solution containing F127-g-gelatin and aCTLA-4 obtained after total disruption of the hydrogel did not show any size relevant to aCTLA-4 (d=9.3±0.6 nm) in dynamic light scattering (DLS) (FIG. 15). Interestingly, aCTLA-4 loaded F127-g-gelatin hydrogels released in situ micelles (d=30.0±1.8 nm), however, which were significantly larger than the in situ micelles from bare F127-g-gelatin hydrogels (d=26.8±3.1 nm) (FIG. 15). These results show that aCTLA-4 can be loaded on in situ F127-g-gelatin micelles. aCTLA-4 loaded F127-g-Gelatin in situ micelles exhibited the size appropriate for efficient lymphatic delivery (10-100 nm), indicating the potential in efficient aCTLA-4 functions in dLN as well as tumor microenvironment.

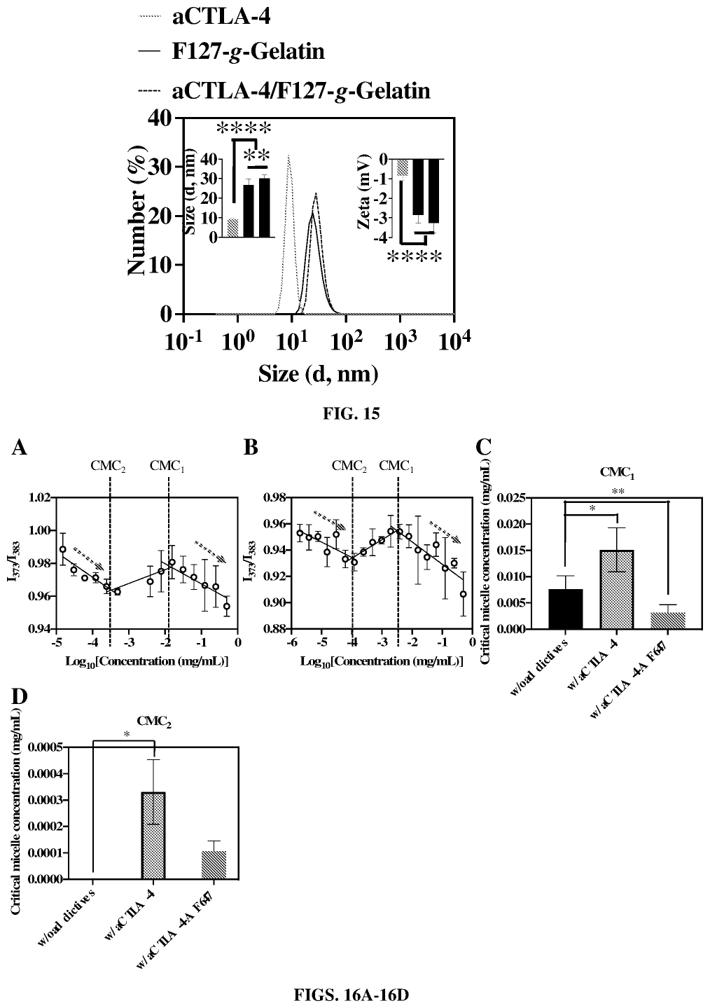

The interactions of aCTLA-4 with F127-g-gelatin in situ micelles were further verified with CMC evaluation. Additional CMC (CMC2) was appeared in F127-g-Gelatin solutions containing aCTLA-4 (FIG. 16, Table 4).

TABLE 4
CMC₁ and CMC₂ of F127-g-Gelatin with or without
aCTLA-4 and aCTLA-4-AF647 at 37° C.

| | | | w/aCTLA-4- |
|---|---|---|---|
| At 37° C. | w/o addictive | w/aCTLA-4 | AF647 |
| CMC₁ (mg mL⁻¹) | 0.0076 ± | 0.015 ± | 0.0031 ± |
| | 0.0026 | 0.0042 | 0.0015 |
| CMC₂ (mg mL⁻¹) | None | 0.00033 ± | 0.00011 ± |
| | | 0.00012 | 0.000039 |

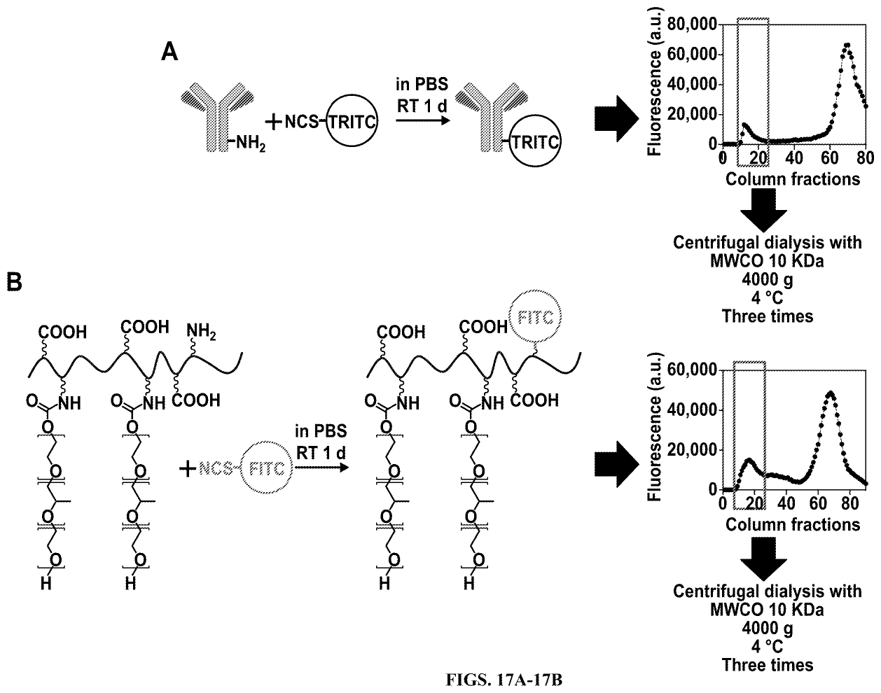

Fluorescence resonance energy transfer (FRET) analysis also confirmed the interactions of aCTLA-4 with F127-g-gelatin in situ micelles. In order to FRET analysis, TRITC labeled aCTLA-4 (aCTLA-4-TRITC) and FITC labeled F127-g-gelatin were synthesized. In brief, 1.8 mg of aCTLA-4 in 200 μL PBS and 20 μL of 1 mg mL⁻¹ TRITC (Thermo Scientific™) in PBS were mixed at room temperature overnight. 8 mg of F127-g-Gelatin in 1 mL PBS and 160 μL of 1 mg mL⁻¹ FITC (Thermo Scientific™) in PBS were mixed at room temperature overnight. CL-6B Sepharose® column (GE Healthcare) and Amicon® Ultra centrifugal filter (Millipore, MWCO 30 kDa) at 4000 g and 4° C. for 20 min were exploited to purify TRITC-labeled aCTLA-4 (aCTLA-4-TRITC) and FITC-labeled F127-g-Gelatin (F127-g-Gelatin-AF647) (FIG. 17). Fluorescence resonance energy transfer (FRET) assay was performed at FITC excitation (495 nm) and TRITC emission (572 nm), which was recorded by Synergy H4 microplate reader (BioTek). aCTLA-4-TRITC itself showed negligible fluorescence. Mixture of aCTLA-4-TRITC and F127-g-gelatin-FITC exhibited the significantly increased fluorescence signal in an aCTLA-4-TRITC concentration-dependent way, compared to F127-g-gelatin-FITC (FIG. 18).

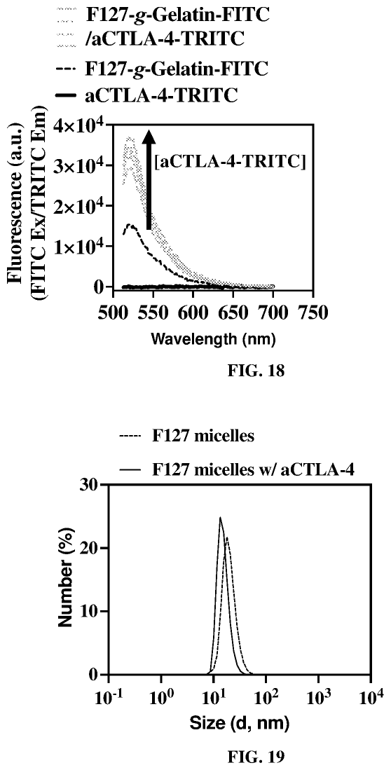

F127 has been reported to interact with proteins including human serum albumin via hydrogen bonding and hydrophobic interactions. Likewise, aCTLA-4 was revealed to bind and incorporate F127 micelles in DLS (FIG. 19). Therefore, F127 blocks in F127-g-gelatin would play an important role in the formation of aCTLA-4 loaded in situ F127-g-gelatin micelles.

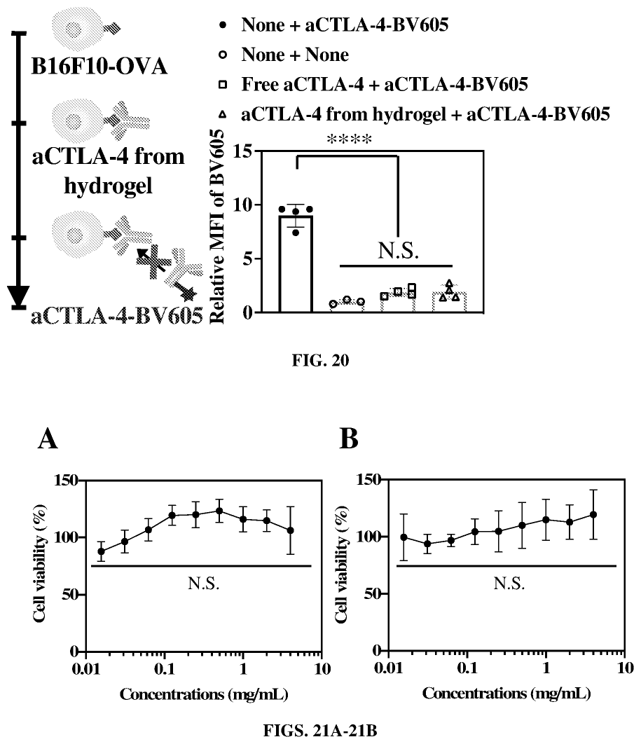

A competitive assay was designed to investigate whether the activity of aCTLA-4 is retained even after released from F127-g-gelatin hydrogel. In brief, aCTLA-4 and 4.5 wt. % F127-g-gelatin containing aCTLA-4 (final aCTLA-4 concentrations equivalent to 0.88 mg mL⁻¹) were prepared in Dulbecco's Modified Eagle Medium (Gibco™, DMEM) containing 10% Fetal Bovine Serum (Gibco™, FBS) and 1× Antibiotic-Antimycotic (Gibco™). They were incubated in 37° C. water incubator until the gels are completely disrupted (4 days). 5×10³ cells well⁻¹ B16F10-OVA were incubated in 96 well U-bottom non-cell culture plates (Falcon®), followed by staining with 2.4G2 (Tonbo bioscience) on ice for 5 min and Zombie Aqua fixable viability dye (Biolegend) at room temperature for 30 min. As-prepared free aCTLA-4 solutions, or aCTLA-4 and F127-g-gelatin hydrogel solutions were treated to the cells for 30 min on ice, followed by incubation with flow cytometry staining buffer (10 mg ml⁻¹ bovine serum albumin (Sigma Aldrich) in PBS, FACS buffer) or aCTLA-4-BV605 (Biolegend, clone: UC10-4B9) in FACS buffer for 30 min on ice. Finally, the cells were fixed with 2% paraformaldehyde in PBS (Alfa Aesar) on ice for 15 min. Cells were washed with PBS or FACS buffers after each step. LSR Fortessa flow cytometry (BD Biosciences) and flowJo (FlowJo LLC) were employed to analyze and profiles the stained cells. Staining of aCTLA-4-BV605 on B16F10-OVA cells was blocked with the pre-treatment of supernatant released from aCTLA-4 loaded F127-g-Gelatin hydrogels at the same level with free aCTLA-4 (FIG. 20). These results demonstrated that the activity of aCTLA-4 was not hampered by F127-g-gelatin in situ micelles despite the interactions between aCTLA-4 and F127-g-gelatin.

### Example #4—In Vitro and In Vivo Biocompatibility Test of F127-g-Gelatin Hydrogels

In vitro biocompatibility of F127-g-gelatin was evaluated. In brief, B 16F10-OVA mouse melanoma and NIH3T3 mouse fibroblast cells were cultured in DMEM containing 10% FBS and 1× Antibiotic-Antimycotic. 10⁴ B16F10-OVA or NIH3T3 (90 μL) seeded in the 96 well cell culture plates were incubated in 37° C. CO₂ incubator overnight. After treating 10 μL of various concentrations of F127-g-gelatin, the cells were incubated in 37° C. CO₂ incubator during 2 days. The cells were incubated with 5 μL of alamarBlue™ cell viability reagent (Invitrogen™) in 37° C. CO₂ incubator for 1 hr, followed by recording fluorescence (560 nm excitation, 590 nm emission) with Synergy H4 microplate reader. F127-g-gelatin did not induce any cytotoxicity on the B16F10-OVA murine melanoma cells and NIH3T3 mouse fibroblast in vitro (FIG. 21).

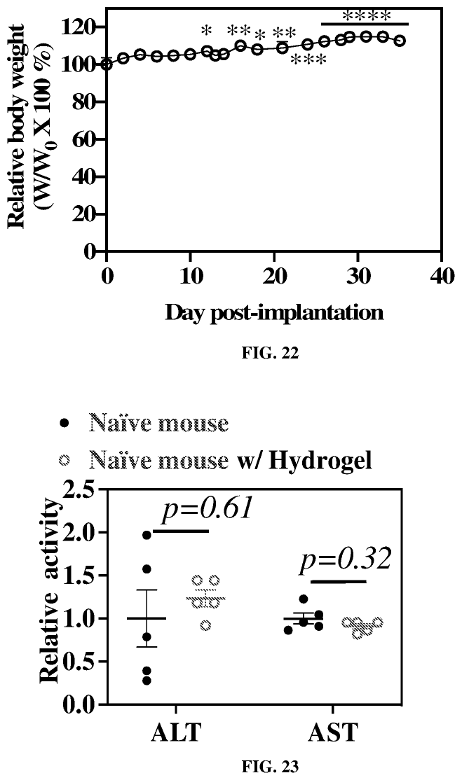

Systemic toxicity was also investigated. F127-g-gelatin hydrogel showed biosafety in terms of body weight changes (FIG. 22). In order to investigate the systemic liver toxicities, Alanine aminotransferase (ALT) activity colorimetry/fluorometry assay (Biovision) and aspartate aminotransferase (AST) activity colorimetric assay (Biovision) were performed on the plasma yielded by two times centrifugation (2100 g, 4° C., 10 min) of blood collected from facial vein 2 day after subcutaneous injection of 4.5 wt. % F127-g-gelatin hydrogels on mice. Any systemic liver toxicity including alanine aminotransferase (ALT) and aspartate aminotransferase (AST) activities was not observed (FIG. 23) in vivo.

### Example #5—In Vivo Biodistribution of aCTLA-4 with aCTLA-4 loaded F127-g-Gelain Micelles and Hydrogel

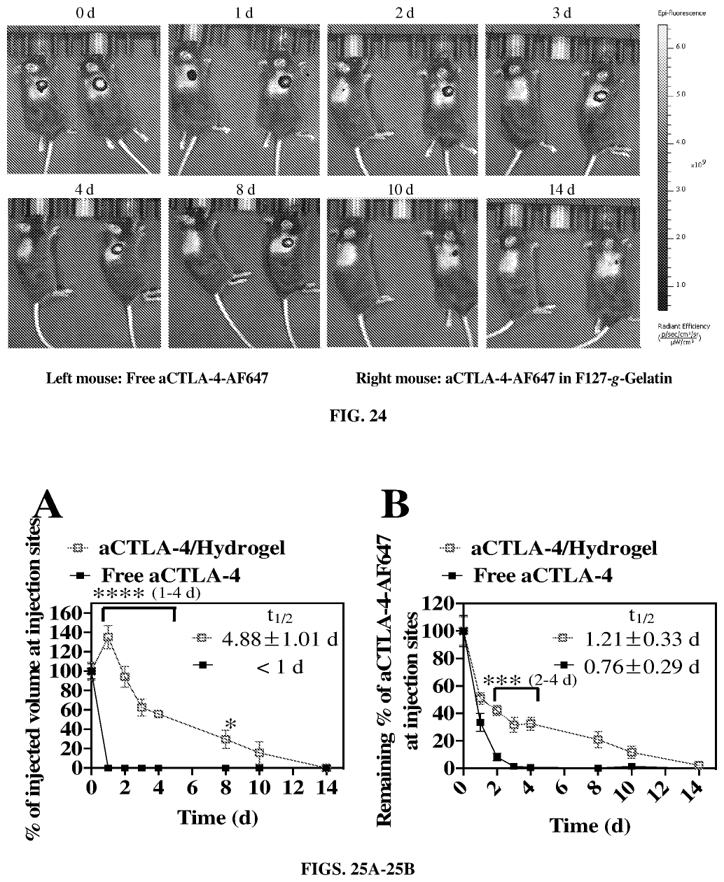

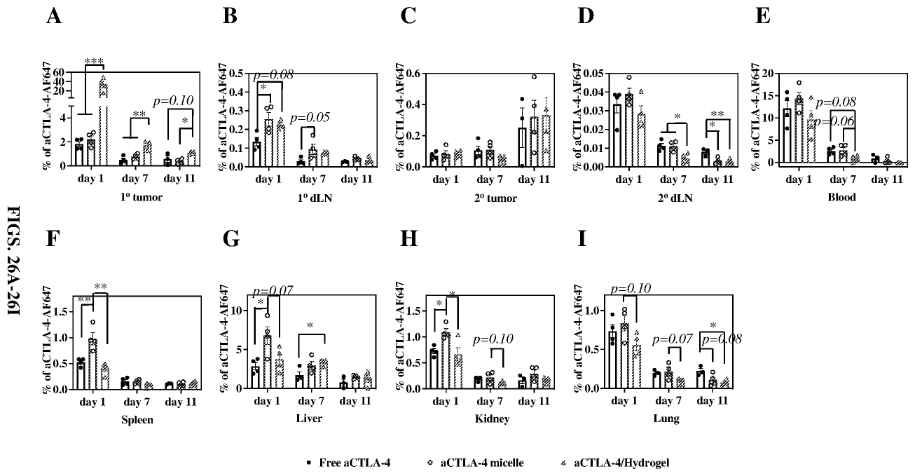

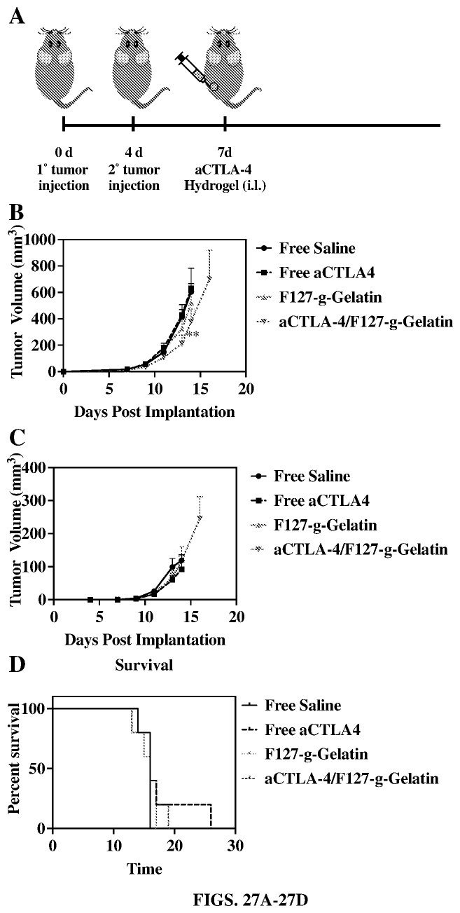

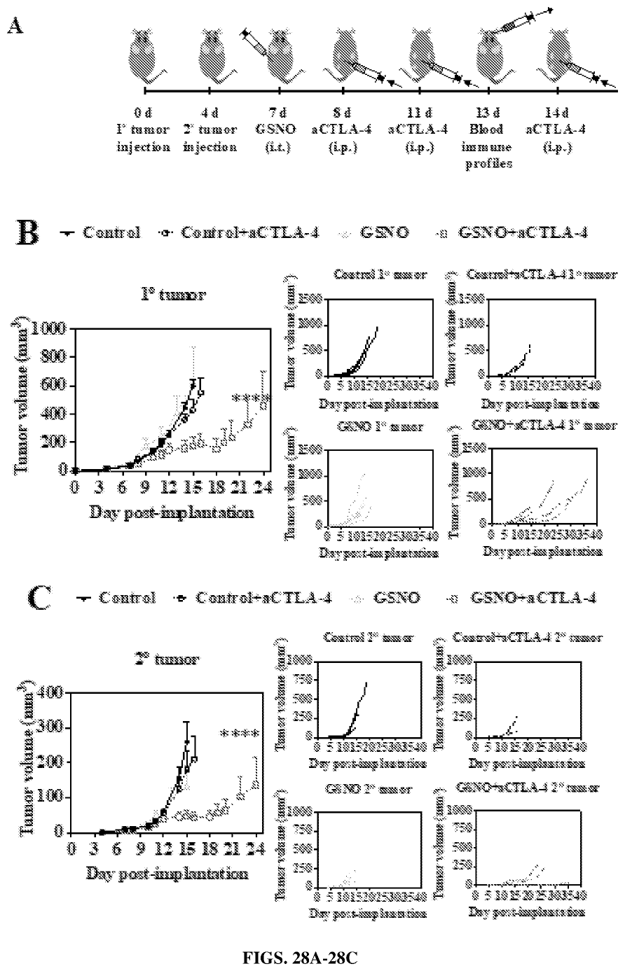

In vivo stability of hydrogel and release of aCTLA-4 were investigated. In brief, the size of free aCTLA-4-AF647 or 4.5 wt. % F127-g-gelatin hydrogels containing aCTLA-4-AF647 (aCTLA-4 dose equivalent to 26.6 μg mouse⁻¹) administered to the left dorsal skin of mice was calculated by a cuboid tube formulation (abc, where a is the length, b is the width, and c is height, respectively). IVIS® Spectrum (Perkin Elmer) was utilized to quantify fluorescence of aCTLA-4-AF647 at the injection site (FIG. 24). The fluorescence represented aCTLA-4-AF647 not released from hydrogel. As observed in the volume and fluorescence signal of free aCTLA-4 and aCTLA-4-AF647 loaded F127-g-gelatin hydrogels, F127-g-gelatin hydrogel facilitates the significantly longer residence time and more sustained release of aCTLA-4 in vivo, compared to bolus delivery (FIG. 25).

The effects of sustained release of aCTLA-4 from hydrogel and the in situ release of micelles containing aCTLA-4 on the biodistribution was investigated. In brief, 10⁵ B16F10-OVA (30 μL) was inoculated in left dorsal skin on day 0 and in right dorsal skin on day 4 to establish dual tumor model. 30 μL of free aCTLA-4-AF647, F127-g-gelatin micelles (0.45 wt %) containing aCTLA-4-AF647, or 4.5 wt. % F127-g-gelatin hydrogels containing aCTLA-4-AF647 (aCTLA-4 dose equivalent to 162 μg mouse⁻¹) was administered to the left tumor on day 7, followed by sacrificing the mice on day 8, 14 and 18 (equivalent to day 1, 7, and 11 after treatment). 1.4 mm zirconium bead filled tubes (OPS Diagnostics) with FastPrep-24 (MP Biomedicals) were used to homogenize the harvested tissues, and the resultant homogenized tissues' fluorescence (650 nm excitation, 670 nm emission) was recorded by Synergy H4 microplate reader. Fluorescence of different concentrations of aCTLA-4-AF647 in the homogenized tissues harvested from untreated tumor-bearing mice was used to establish standard curves of aCTLA-4-AF647 for each tissue. Owing to the sustained release of aCTLA-4 from F127-g-gelatin hydrogel, intratumoral administrations of aCTLA-4-AF647 loaded F127-g-Gelatin hydrogels facilitated significantly prolonged and higher tumoral accumulations of aCTLA-4 (FIG. 26A) compared to free aCTLA-4-AF647 and aCTLA-4-AF647 loaded F127-g-gelatin micelles. Like F127-g-gelatin micelles, higher dLN accumulation of aCTLA-4 was observed with the F127-g-gelatin hydrogels compared to bolus delivery (FIG. 26B), which is attributed to the in situ release of aCTLA-4 loaded F127-g-gelatin micelles appropriate for the dLN delivery. These results indicated that F127-g-gelatin hydrogels not only facilitate the sustained intratumoral accumulations of aCTLA-4, but also enable the efficient delivery of aCTLA-4 into dLN. In addition, F127-g-gelatin hydrogel reduced systemic exposure of aCTLA-4 to other tissues (FIG. 26C-I) compared to free aCTLA-4 or aCTLA-4 loaded F127-g-gelatin micelles, implying the potential ability of F127-g-Gelatin hydrogel in reducing the ICBs-associated systemic immune-related adverse events.

### Example #6—In Vivo Therapeutic Effects with F127-g-Gelatin Hydrogel in Single aCTLA-4 Therapy

Melanoma including B16F10-OVA is well-known to be resistant to single aCTLA-4 therapy. Therefore, it was investigated whether F127-g-gelatin hydrogel improves the therapeutic effects of aCTLA. In brief, 30 μL of 10⁵ B16F10-OVA was inoculated in left dorsal skin on day 0 and in right dorsal skin on day 4 to establish dual tumor model, and then 30 μL of saline, free aCTLA-4, 4.5 wt. % F127-g-gelatin hydrogel, or aCTLA-4 loaded 4.5 wt. % F127-g-gelatin hydrogels (aCTLA-4 dose equivalent to 300 μg mouse⁻¹) was administered via intradermal (i.d.) injection to the tissue ipsilateral (i.l.) to the tumor on day 7 (FIG. 27). Although free delivery of aCTLA-4 did not show any therapeutic effects compared to saline groups, aCTLA-4 with F127-g-gelatin hydrogel (aCTLA-4/F127-g-gelatin) led to significantly enhanced therapeutic effects (FIG. 27B). This intradermal (i.d.) injection to the tissue ipsilateral (i.l.) to the 1º tumor strategy did not lead to systemic therapeutic effects as shown in 2º tumor with negligible antitumor effects on all groups (FIG. 27C), indicating that the intradermal (i.d.) injection of immune checkpoint blockade antibodies to the tissue ipsilateral (i.l.) to the tumor strategy with F127-g-Gelatin hydrogel facilitates the enhancement of local immunotherapy.

### Example #7—In Vivo Therapeutic Effects with F127-g-Gelatin Hydrogel in aCTLA-4+GSNO Therapy

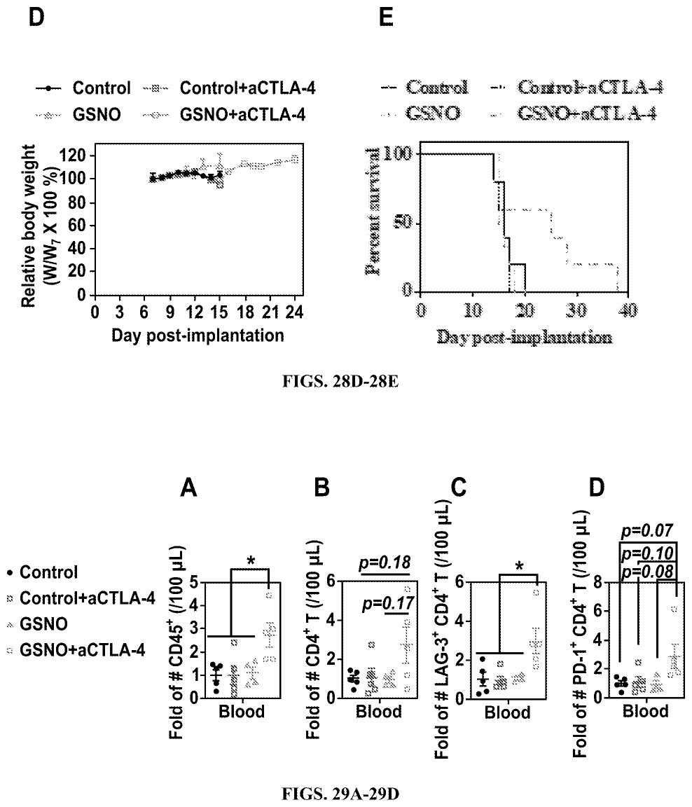

It was investigated in a dual B16F10-OVA tumor model whether the delivery of each GSNO, aCTLA-4, and GSNO+aCTLA-4 leads to anticancer effects without F127-g-gelatin hydrogel (FIG. 28). In brief, 10⁵ B16F10-OVA (30 μL) was inoculated in left dorsal skin on day 0 and in right dorsal skin on day 4 to establish dual tumor model. 30 μL of saline or GSNO (480 μg kg ⁻¹) was administered to the left tumor on day 7 and 30 μL of aCTLA-4 (100 μg mouse⁻¹) was administered intraperitoneally on day 8, 11, 14. The tumor size was calculated by a cuboid tube formulation (abc, where a is the length, b is the width, and c is height, respectively). Each GSNO and aCTLA-4 did not lead to any therapeutic effects compared to saline (FIG. 28). Surprisingly, the combinational use of GSNO and aCTLA-4 not only significantly reduced the growth of both 1º and 2º tumor, but also prolonged the animal survival with negligible weight changes (FIG. 28).

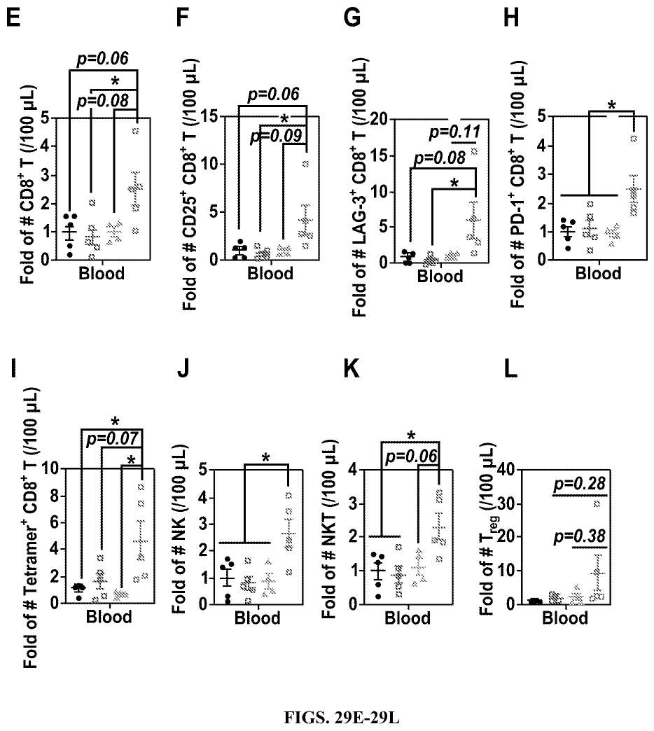

Immune cells in blood were collected and profiled to delineate the systemic immunity in the combinational use of GSNO and aCTLA-4 (FIG. 29). In brief, ACK lysis buffer was treated to the blood taken on day 9 to harvest immune cells in blood by removing red blood cells. All cells were stored on ice<2 h prior to use. Cells for flow cytometry were prepared by six steps with PBS, FACS buffer, or permeabilization buffer (eBioscience™ Foxp3/Transcription Factor Staining Buffer Set, Invitrogen™) wash; 2.4G2 staining on ice for 5 min, Zombie Aqua fixable viability dye staining at room temperature for 30 min, SIINFEKL-MHCI-PE tetramer (NIH Tetramer Core Facility, Atlanta, Georgia) staining on ice for 15 min, antibody mixtures staining on ice for 30 min, fixing and permeabilizing with Foxp3 Fixation/Permeabilization working solution (eBioscience™ Foxp3/Transcription Factor Staining Buffer Set, Invitrogen™) on ice for 60 min, and FoxP3 staining on ice for 75 min. LSR Fortessa flow cytometry and flowJo were employed to analyze and profiles the stained cells. The antibody information is listed in the Table 5. GSNO and aCTLA-4 failed to induce systemic activation and expansion of T cell, NK and NKT response. However, Significant expansion of CD4⁺T, CD8⁺T, CD3⁻NK1.1⁺ (NK), and CD3⁺NK1.1⁺ (NK T cells, NKT) was observed in blood, indicating that the systemic synergistic anticancer effects of GSNO and aCTLA-4 are attributed to the robust systemic T cell, NK and NKT response (FIG. 29). In particular, the population of LAG-3⁺CD4⁺T, PD-1⁺CD4⁺T, CD25⁺CD8⁺T, LAG-3⁺CD8⁺T, and PD-1⁺CD8⁺T was significantly expanded with Tetramer+CD8⁺T. Considering that CD25 and PD-1 on T cells are representative makers for antigen-experienced T cells and LAG-3 is expressed on activated T cells, these results clearly demonstrated that the combinational use of GSNO and aCTLA-4 facilitates the robust improved antigen-specific T cell prime by Dendritic cells (DCs).

TABLE 5
Antibody list for immune cell profiles in blood.

| Color | Antibody | Clone | Company |
|---|---|---|---|
| FITC | CD4 | GK1.5 | Biolegend |
| PerCP | CD45 | 30-F11 | Biolegend |
| PE | SIINFEKL-MHCI-PE | | NIH Tetramer |
| | | | Core Facility |
| APC | LAG-3 | C9B7W | Biolegend |
| AF700 | CD25 | PC61 | Biolegend |
| APC/Cy7 | CD8 | 53-6.7 | Biolegend |
| BV421 | FoxP3 | MF-14 | Biolegend |
| BV605 | NK1.1 | PK136 | Biolegend |
| BV711 | CD3 | 145-2C11 | Biolegend |
| BV785 | PD-1 | 29F.1A12 | Biolegend |
| Zombie Aqua | | | Biolegend |

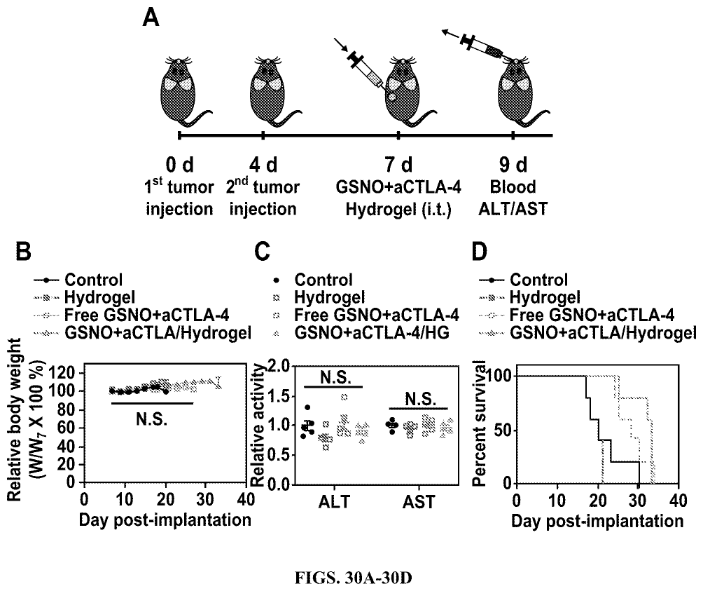

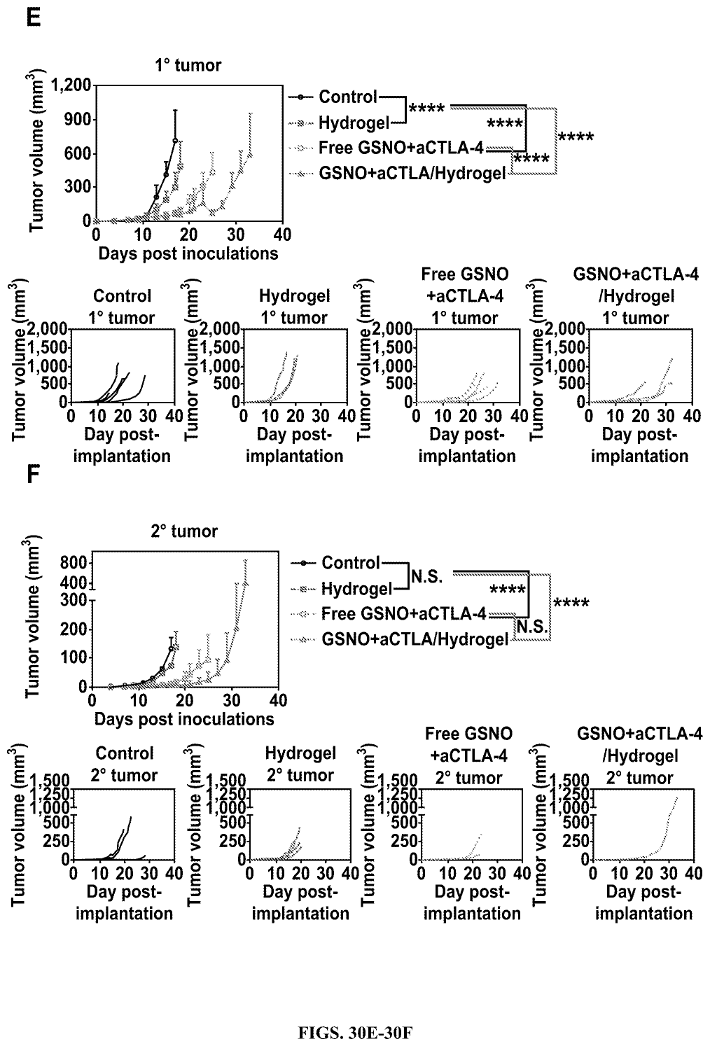

It was investigated in a dual B16F10-OVA tumor model whether the delivery of GSNO and aCTLA-4 with F127-g-gelatin hydrogel (GSNO+aCTLA-4/Hydrogel) leads to more efficient and durable systemic therapeutic effects than bolus delivery (FIG. 30A). In brief, 10⁵ B16F10-OVA (30 μL) was inoculated in left dorsal skin on day 0 and in right dorsal skin on day 4 to establish dual tumor model. 30 μL of 4.5 wt. % F127-g-gelatin hydrogel containing GSNO (570 μg kg⁻¹) and aCTLA-4 (100 μg mouse⁻¹), or 30 μL of GSNO (570 μg kg⁻¹) and aCTLA-4 (100 μg mouse⁻¹) in saline was administered to the left tumor on day 7. Blood was collected from facial vein on day 9 for blood ALT/AST assay. The intratumoral administrations of GSNO+aCTLA-4/Hydrogel showed negligible changes of body weights and ALT/AST activities (FIG. 30B,C), demonstrating its systemic biosafety. Intratumoral GSNO+aCTLA-4/Hydrogel not only prolonged the animal survival, but also led to significantly more durable and efficient antitumor effects in both 1º and 2º tumor, compared to intratumoral bolus delivery as well as saline and bare F127-g-gelatin hydrogels (FIG. 30D-F). The improved therapeutic index with F127-g-gelatin hydrogel would be due to the durable actions of GSNO and aCTLA-4 in tumor microenvironment as well as dLN.

### Example #8—In Vitro Residence Stability and Release of Vemurafenib (Vem) and aPD-1 from Hydrogel

TRITC labeled aPD-1 (aPD-1-TRITC) was prepared for the drug release test. In brief, 3.36 mg of aPD-1 (BioXCell, clone: RMP1-14) in 400 μL PBS was reacted with 66 μL of 1 mg mL⁻¹ TRITC in DMSO at room temperature for 2 h. TRITC-labeled aPD-1 (aPD-1-AF647), was purified using Zeba desalting column (Thermo Scientific™, MWCO 7K) 3 times with manufacturer's instruction.

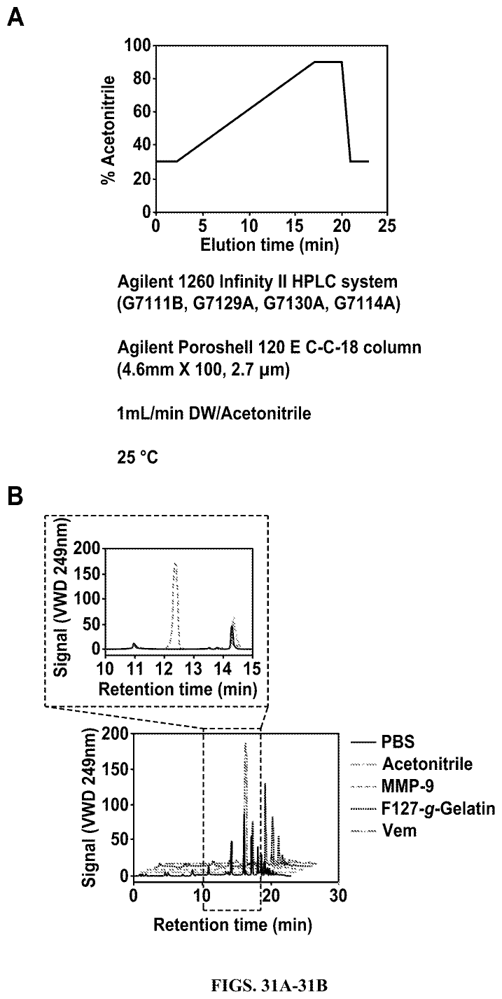

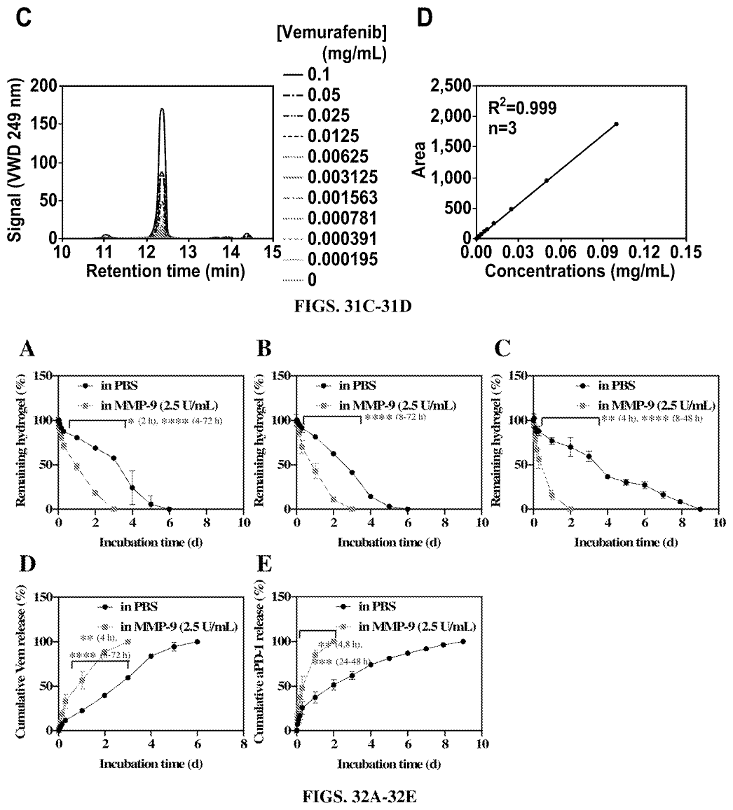

300 μL of F127-g-Gelatin 4.5 wt. % hydrogels containing Vem (LC laboratories) (final Vem concentrations equivalent to 0.67 mg mL⁻¹) or aPD-1-TRITC (final aPD-1 concentrations equivalent to 0.672 mg mL⁻¹) were prepared in 1.5 mL e-tube in 37° C. water incubator and then incubated with additional 300 μL of PBS or 2.5 U mL⁻¹ MMP-9 (Gibco™ collagenase IV). After sampling the supernatants at predetermined time intervals, the remaining mass of the hydrogels were recorded to investigate the residence stability of the hydrogels, and then fresh 300 μL of PBS or 2.5 U mL⁻¹ MMP-9 was added to the e-tube. Vem release was evaluated using gradient reverse phase high-pressure liquid chromatography (HPLC) of the supernatant samples yielded at predetermined time intervals. The detailed conditions for HPLC are described in FIG. 31. aPD-1 release was quantified by recording TRITC fluorescence (547 nm excitation, 579 nm emission) of supernatant samples yielded at predetermined time intervals with Synergy H4 microplate.

F127-g-gelatin hydrogels containing aPD-1-TRITC exhibited the prolonged residence time in vitro compared to bare F127-g-gelatin hydrogels and F127-g-gelatin hydrogels containing Vem (FIG. 32A-C), implicating the association of aPD-1 in the formation of F127-g-gelatin hydrogels. As a result, F127-g-gelatin hydrogels containing aPD-1-TRITC exhibited the prolonged release half-life in vitro compared to bare F127-g-gelatin hydrogels and F127-g-gelatin hydrogels containing Vem (FIG. 32D-E). Indeed, the addition of Vem did not affect the CMC measured by pyrene method, whereas aPD-1-TRITC depicted F127-g-gelatin micelle showed the concentration-dependent V-shape graph associated with self-quenching of TRITC in the nanoparticles (FIG. 33A-C).

TABLE 6
Summary of CMC of F127-g-gelatin with or without
aCTLA-4 and aCTLA-4-AF647 at 37° C.

| | CMC (mg mL⁻¹) | CMC (mg mL⁻¹) |
|---|---|---|
| | at RT | at 37° C. |
| Pyrene/F127-g-gelatin | 0.20 ± 0.04 | 0.028 ± 0.009 |
| Pyrene + Vem/F127-g-gelatin | 0.22 ± 0.08 | 0.021 ± 0.007 |

### Example #9—In Vivo Biodistribution of aPD-1 with aPD-1 and Vem Loaded F127-g-Gelatin Hydrogel

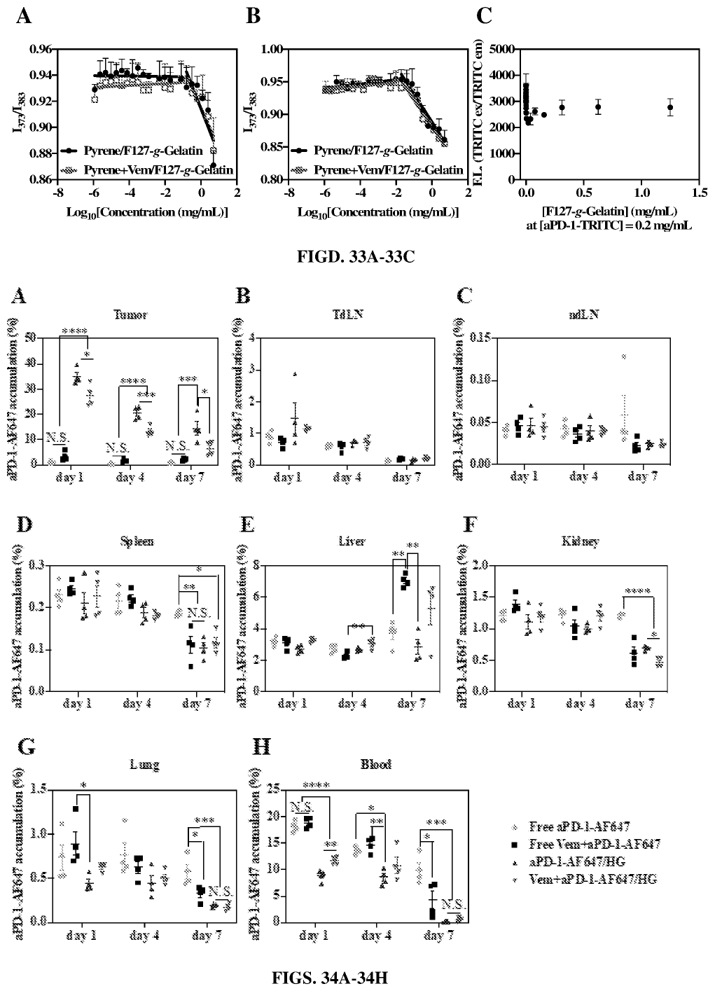

The effects of sustained release of aPD-1 from hydrogel and co-delivery of Vem in aPD-1 releasing hydrogel on the biodistribution of aPD-1 was investigated. In brief, aPD-1-AF647 was prepared by reacting 15.7 mg of aPD-1 (BioXCell, clone: RMP1-14) in 1.2 mL PBS with 30 μL of 5 mg mL⁻¹ Alexa Fluor™ 647 NHS Ester (AF647-NHS) (Invitrogen™) in DMSO at room temperature for 2 h, followed by purification with Zeba desalting column (Thermo Scientific™, MWCO 7K) 5 times. 30 μL of 5×10⁵ D4M, a BRAF-mutated melanoma cell line, was inoculated in left dorsal skin on day 0 to establish tumor model, and then 30 μL of free aPD-1-AF647, free Vem+aPD-1-AF647, aPD-1-AF647 with 4.5 wt. % F127-g-gelatin hydrogel, and Vem+aPD-1-AF647 with 4.5 wt. % F127-g-gelatin hydrogel (aPD-1 and Vem dose equivalent to 100 μg mouse⁻¹, and 10 mg kg⁻¹, respectively) was administered to the tumor on day 7. Mice were sacrificed on day 8, 11 and 14 (equivalent to day 1, 4, and 7 after treatment). Harvested tissues were homogenized in 1.4 mm zirconium bead filled tubes (OPS Diagnostics) with FastPrep-24 (MP Biomedicals), and the fluorescence (650 nm excitation, 670 nm emission) was recorded by Synergy H4 microplate reader. Standard curves of aPD-1-AF647 for each tissue were established by recording fluorescence of different concentrations of aPD-1-AF647 added homogenized tissues harvested from untreated tumor-bearing mice (FIG. 34). Owing to the sustained release of aPD-1 from F127-g-gelatin hydrogel, intratumoral administrations of aPD-1-AF647 loaded F127-g-gelatin hydrogels facilitated significantly prolonged and higher tumoral accumulations of aPD-1 (FIG. 34A) compared to free aPD-1-AF647. As a result, systemic exposure of aPD-1 was significantly reduced, as shown in spleen (FIG. 34D), kidney (FIG. 34F), Lung (FIG. 34G), and Blood (FIG. 34H). As contrasted with the aCTLA-4, slight increase of aPD-1 accumulations in tumor draining lymph nodes (TdLN) was observed (FIG. 34B) when directly comparing Vem+aPD-1-AF647/HG with Free Vem+aPD-1-AF647 or comparing aPD-1-AF647/HG with Free aPD-1-AF647 with student's t-test. Interestingly, Vem reduced the accumulations of aPD-1 in the tumor, which is attributed to the improved efflux by Vem-mediated enhanced perfused functions of blood vessel, as reported previously. As a result, Vem increased the accumulations of aPD-1 in liver in both bolus and hydrogel delivery.

### Example #10—In Vivo Therapeutic Effects with F127-g-Gelatin Hydrogel in Vem+aPD-1 Therapy

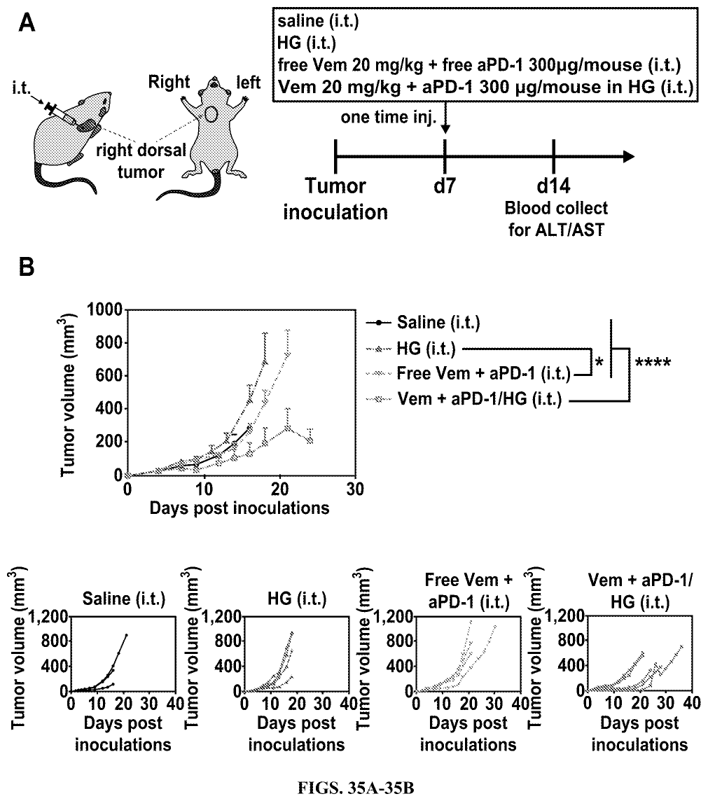

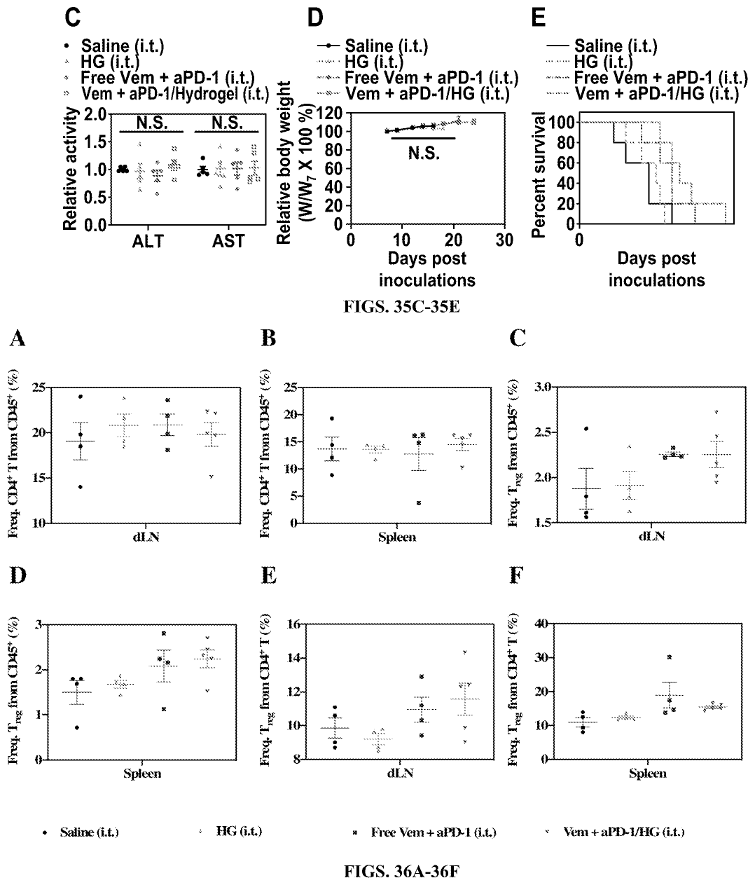

It was investigated in a D4M tumor model whether the delivery of Vem and aPD-1 with F127-g-gelatin hydrogel (Vem+aPD-1/Hydrogel) leads to more efficient therapeutic effects than bolus delivery (FIG. 35A). In brief, 30 μL of 5×10⁵ DM4 was inoculated in left dorsal skin on day 0, and then 30 μL of 4.5 wt. % F127-g-gelatin hydrogel containing Vem (20 mg kg⁻¹) and aPD-1 (300 μg mouse⁻¹) or 30 μL of Vem (20 mg kg⁻¹) and aPD-1 (300 μg mouse⁻¹) in 20% DMSO in saline was administered to the left tumor on day 7. Blood was collected from facial vein on day 14 for blood ALT/AST assay. Intratumoral Vem+aPD-1/Hydrogel not only prolonged the animal survival, but also led to significantly more durable and efficient antitumor effects on tumor, compared to intratumoral bolus delivery as well as saline and bare F127-g-Gelatin hydrogels (FIG. 35B). The intratumoral administrations of Vem+aPD-1/Hydrogel showed negligible changes of ALT/AST activities and body weights and (FIG. 35C,D), demonstrating its systemic biosafety. As a result, enhanced survival was observed in Vem+aPD-1/HG, compared to intratumoral bolus delivery as well as saline and bare F127-g-Gelatin hydrogels (FIG. 35E).

### Example 11—In Vivo Immune Phenotyping of Mice Treated with Vem+aPD-1 Containing F127-g-Gelatin Hydrogel

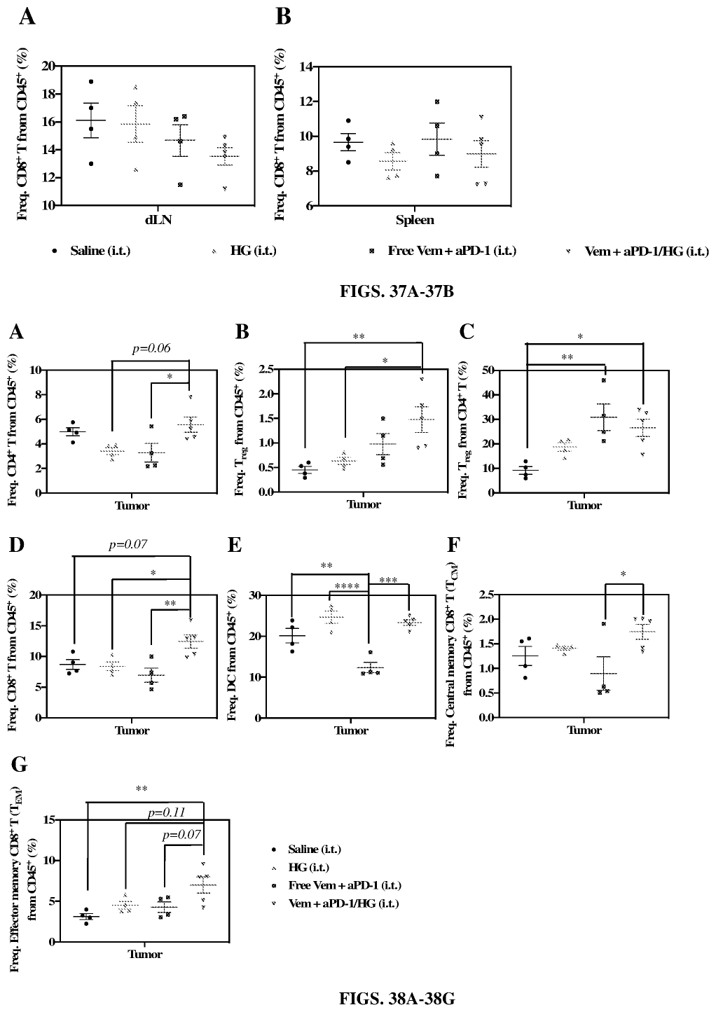

Immune cells in tumor draining lymph node (dLN), spleen, and tumor were profiled to delineate the immunity in Vem+aPD-1/HG (FIG. 36). In brief, 30 μL of 5×10⁵ DM4 was inoculated in left dorsal skin on day 0, and then 30 μL of 4.5 wt. % F127-g-gelatin hydrogel containing Vem (20 mg kg⁻¹) and aPD-1 (300 μg mouse⁻¹) or 30 μL of Vem (20 mg kg⁻¹) and aPD-1 (300 μg mouse⁻¹) in 20% DMSO in saline was administered to the left tumor on day 7. Mice were sacrificed and tumor draining lymph nodes, spleen, and tumor were collected on day 14 for immune cell profiles. Immune cells were harvested by incubating in gelatinase D (45 min for lymph node, and 3 hr for tumor) or by removing red blood cells (for spleen) with ACK lysis buffer. All cells were stored on ice<2 h prior to use. Cells for flow cytometry were prepared by staining with 2.4G2 on ice for 5 min, staining with Zombie Aqua fixable viability dye at room temperature for 30 min, staining with antibody mixtures on ice for 30 min, fixing and permeabilizing with Foxp3 Fixation/Permeabilization working solution (eBioscience™ Foxp3/Transcription Factor Staining Buffer Set, Invitrogen™) on ice for 60 min, and staining FoxP3 and Tcf1 on ice for 75 min. Cells were washed with PBS, FACS buffer, or permeabilization buffer (eBioscience™ Foxp3/Transcription Factor Staining Buffer Set, Invitrogen™) after each step. LSR Fortessa flow cytometry and flowJo were employed to analyze and profiles the stained cells (FIG. 36-41). The antibody information is listed in the Table 7.

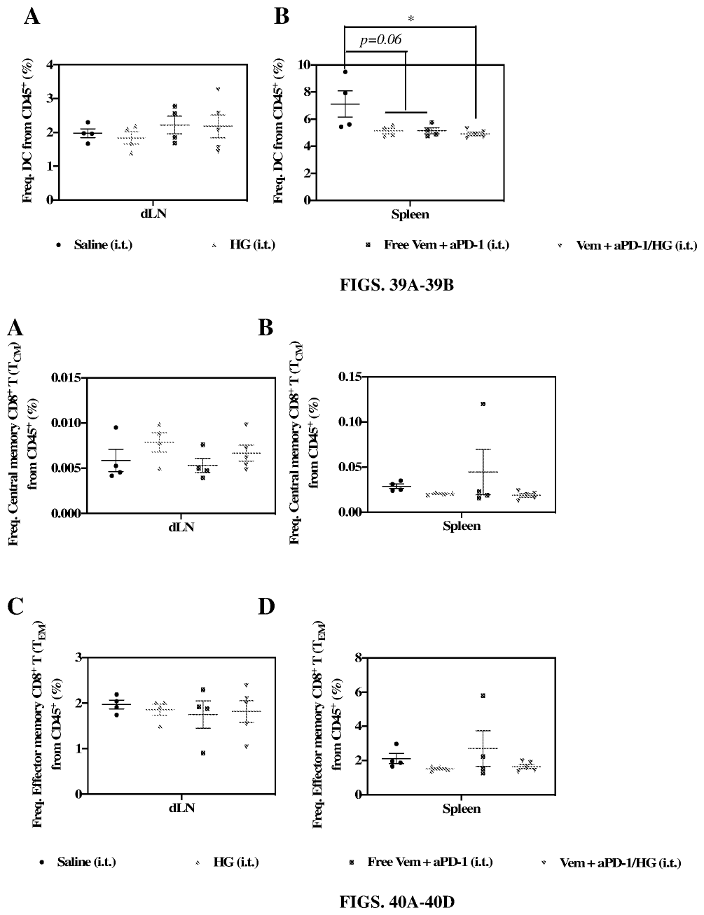

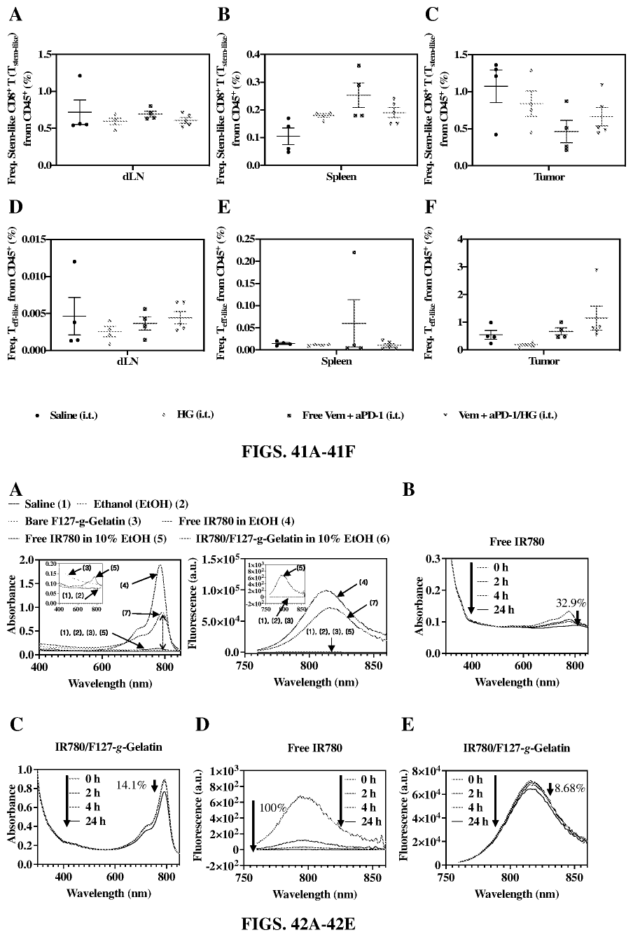

There were no statistical differences of CD4⁺T, Foxp3⁺CD4⁺ regulatory T cell (CD45⁺CD4⁺Foxp3⁺, T_{reg}), and CD8⁺T in dLN and spleen (FIG. 36,37). However, the population of CD4⁺T in Vem+aPD-1/HG was significantly increased, compared to bare HG and free Vem+aPD-1 (FIG. 38A). T_{reg} was significantly increased in Vem+aPD-1/HG, compared to Saline and bare HG, but the difference of T_{reg} populations between Free Vem+aPD-1 and Vem+aPD-1/HG was negligible (FIG. 38B,C). In particular, the population of CD8⁺T was significantly higher in Vem+aPD-1/HG than in saline, bare HG, and Free Vem+aPD-1 (FIG. 38D). Interestingly, populations of dendritic cells in Free Vem+aPD-1 were significantly reduced compared to saline, bare HG, and Vem+aPD-1/HG, although there were no statistical differences between saline, bare HG, and Vem+aPD-1/HG (FIG. 38E). Although the population of DC in dLN were not different among samples (FIG. 39A), it was decreased in bare HG, Free Vem+aPD-1, and Vem+aPD-1/HG, compared to saline groups (FIG. 39B). Accordingly, it was speculated that there are other mechanisms to augment CD8⁺T expansion and infiltration in the tumor, such as proliferations of memory T cells or stem-like T cells. Surprisingly, central memory CD8⁺T (T_{CM}) and effector memory CD8⁺T (T_{EM}) were significantly expanded in the tumor (FIG. 38F,G), while there are no changes in the dLN and spleen (FIG. 40). However, stem-like CD8⁺T (Tₛₜₑₘ₋ₗᵢₖₑ), and effector-like CD8⁺T (T_{eff-like}) among samples were not changed in dLN, spleen, and tumor (FIG. 41).

In conclusion, compared to bolus delivery, F127-g-gelatin thermosensitive HG system led to the enhanced expansion and infiltration of CD8⁺T cells in the tumor by preventing DC depletion and invigorating Tcm and TEM in the tumor.

TABLE 7
Antibody list for immune cell profiles.

| Color | Antibody | Clone | Company |
|---|---|---|---|
| FITC | CD4 | GK1.5 | Biolegend |
| PerCP | CD45 | 30-F11 | Biolegend |
| PE | Tcf1 | S33-966 | BD Science |
| PE/Cy7 | CD11c | N418 | Biolegend |
| APC | PD-1 | 29F.1A12 | Biolegend |
| AF700 | CD62L | MEL-14 | Biolegend |
| APC/Cy7 | CD8 | 53-6.7 | Biolegend |
| BV421 | Foxp3 | MF-14 | Biolegend |
| BV605 | Tim-3 | RMT3-23 | Biolegend |
| BV711 | CD3 | 145-2C11 | Biolegend |
| BV785 | CD44 | IM7 | Biolegend |

### Example 12—In Vivo Image Guided Surgery with IR780 Dyes Loaded F127-g-Gelatin Hydrogel

4.5 wt. % F127-g-Gelatin was solubilized with IR780 iodide in 10% ethanol/saline solution. The absorption and fluorescence spectra (740 nm excitation) of IR780 solutions and IR780/F127-g-Gelatin solutions were measured using a Synergy H4 microplate reader (BioTek) after being exposed to daylight. IR780 dyes in 10% ethanol showed the lower absorbance and fluorescence intensity than those in 100% ethanol because hydrophobic IR780 is poorly solubilized, aggregated and self-quenched in 10% ethanol (FIG. 42A). However, F127-g-Gelatin hydrogel recovered the absorbance and fluorescence of IR780 solubilized in 10% ethanol by improving the solubility of IR780 (FIG. 42A). While free IR780 in 10% ethanol was completely photobleached within 24 h, F127-g-Gelatin hydrogels significantly reduced the photobleaching of IR780 by preventing dyes from being directly exposed to the light (FIG. 42B-E).

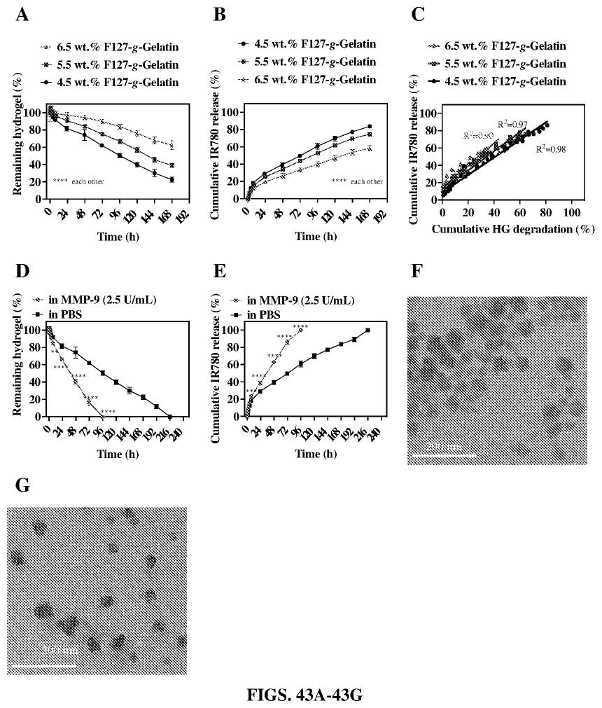

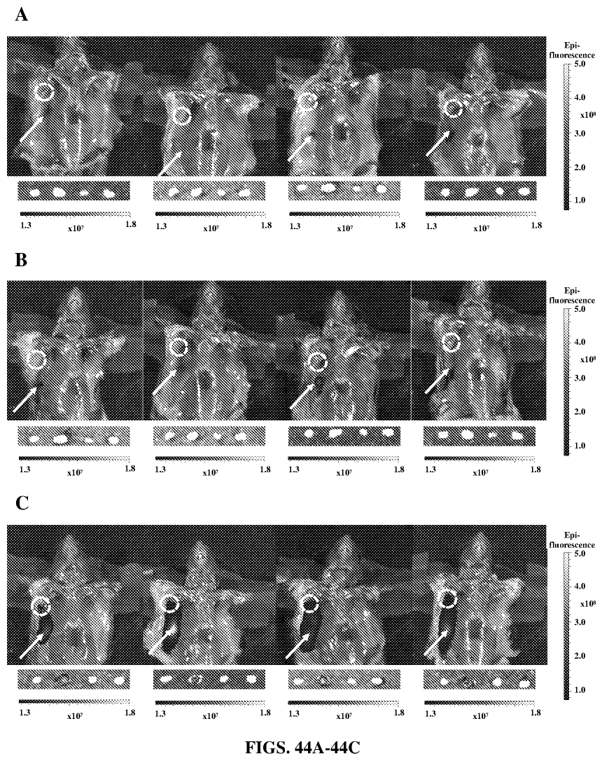

300 μL of F127-g-Gelatin 4.5, 5.5, and 6.5 wt. % hydrogel containing IR780 (final IR780 concentrations equivalent to 0.02 mg mL⁻¹ in 10% ethanol) was prepared in 1.5 mL e-tube in 37° C. water incubator and then incubated with additional 300 μL of PBS or 2.5 U mL-1 MMP-9 (Gibco™, collagenase IV). After sampling the supernatants at predetermined time intervals, the remaining mass of the hydrogels were recorded to investigate the residence stability of the hydrogels, and then fresh 300 μL of PBS or 2.5 U mL-1 MMP-9 was added to the e-tube. IR780 release was evaluated using a Synergy H4 microplate reader to measure IR780 (740 nm excitation and 760 nm emission) of the supernatant samples yielded at predetermined time intervals. The degradation of IR780 dyes loaded F127-g-Gelatin hydrogels was dependent on the concentrations of the hydrogels (FIG. 43A). F127-g-Gelatin facilitated the sustained release of IR780 dyes (FIG. 43B). In particular, IR780 release profiles were correlated with the degradation of F127-g-Gelatin hydrogels (FIG. 43C). Due to the MMP-9 sensitive degradation of F127-g-Gelatin hydrogels (FIG. 43D), IR780 release also depends on the MMP-9 (FIG. 43E). In addition, F127-g-Gelatin hydrogels released in situ micelles loading IR780 (FIG. 43F,G).

It was investigated in a B16F10-OVA tumor model whether F127-g-Gelatin hydrogels improved the detection of TdLNs and tumors (FIG. 44). C57B1/6 mice were obtained from Jackson Laboratories. 10⁵ B16F10-OVA cells in 30 μL of saline were implanted in the right dorsal skin of mice on day 0, IR780 (15 μL of 0.016 mg mL⁻1) with or without F127-g-Gelatin hydrogel (4.5 wt. %) was injected to the tumor on day 7, and then IVIS images was obtained 24 h after injection (FIG. 44). While free formulations of IR780 cleared quickly from the injected tumor tissues and exhibited negligible signals in the TdLNs (FIG. 44B), F127-g-Gelatin hydrogels facilitated the detection of the injected tumor tissues as well as TdLNs (FIG. 44C). These results would be attributed to the size of nanoparticles. 20-200 nm nanoparticles exhibited lower extravasation to the tumor through blood vessels than small molecules (<10 nm). However small molecules (<10 nm) are more easily cleared from tumor tissues via tumor vascular perfusion than nanoparticles (20-200 nm). Accordingly, size should be optimized to be 20-200 nm in efficient tumoral accumulations. On the other hands, size thresholds of extracellular matrix and size-dependent diffusion and bulk fluid flow in lymphatic systems allow 10-100 nm nanoparticles (especially, 30 nm organic nanoparticles) to be most efficient in lymphatic accumulations. As the size of in situ micelles released from F127-g-Gelatin is about 30 nm that is the appropriate size in both tumoral and lymphatic accumulations, F127-g-Gelatin allowed the efficient detection of tumor and lymph nodes simultaneously. Indeed, F127-g-Gelatin hydrogel facilitated the dissected brachial TdLNs that is primary TdLNs of this tumor model to exhibit significantly higher IR780 signals than free formulation, clearly suggesting the potential usage of F127-g-Gelatin hydrogel platforms in image-guided surgery.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A thermosensitive hydrogel comprising at least one thermosensitive polymer and at least one polypeptide, wherein the thermosensitive polymer has a first portion that is covalently crosslinked to at least one polypeptide, wherein the ratio of thermosensitive polymer:polypeptide is from 5:1 to 1:1, wt./wt., wherein the thermosensitive polymer comprises a block copolymer comprising at least one block of polyethylene glycol, and at least one block of a polypropylene glycol or polyester; and
the polypeptide comprises gelatin.

2-3. (canceled)

4. The thermosensitive hydrogel according to claim 1, wherein the thermosensitive polymer comprises a second portion wherein the thermosensitive polymer is singly linked to the polypeptide, wherein the first portion of the thermosensitive polymer that is covalently crosslinked to the polypeptide is present in an amount at least 90%, relative to the total amount of thermosensitive polymer in the hydrogel.

5-12. (canceled)

13. The thermosensitive hydrogel according to claim 1, wherein the thermosensitive polymer comprises a branched block copolymer having the formula:
[core]-[polypropylene glycol-polyethylene glycol]ₓ, [core]-[polyethylene glycol-poly (lactic-co-glycolic acid]ₓ, [core]-[poly(lactic-co-glycolic acid-polyethylene glycol]ₓ, [core]-[polylactic acid-polyethylene glycol]ₓ, [core]-[polyethylene glycol-polylactic acid]ₓ, [core]-[polycaprolactone-polyethylene glycol]ₓ, [core]-[polyethylene glycol-poly (3-hydroxybutyrate]ₓ, [core]-[poly(3-hydroxybutyrate-polyethylene glycol]ₓ, wherein x is 3, 4, 5, 6, 7, or 8, and [core] has the formula: 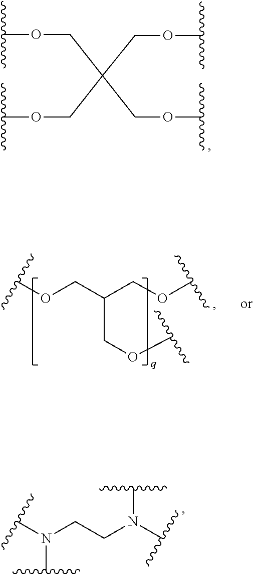
wherein q is 1, 2, 3, 4, 5, or 6, and each wavy line represents a bond to a thermosensitive polymer chain.

14-32. (canceled)

33. The thermosensitive polymer according to claim 1, wherein the first portion of the thermosensitive polymer has the structure: 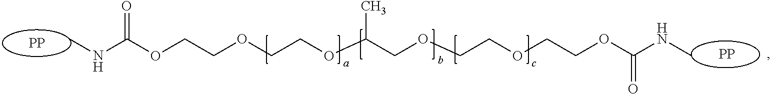
wherein a, b, and c are each independently selected from 1-1,000.

34. (canceled)

35. The thermosensitive hydrogel according to claim 1, further comprising at least one therapeutic or diagnostic agent.

36. The thermosensitive hydrogel according to claim 1, further comprising metallic nanoparticles.

37-39. (canceled)

40. The thermosensitive hydrogel according to claim 35, comprising a NO-donor, immunomodulator, anti-cancer agent, or a combination thereof.

41. (canceled)

42. The thermosensitive hydrogel according to claim 35, comprising a nucleoside analogue, antifolate, antimetabolite, topoisomerase I inhibitor, anthracycline, podophyllotoxin, taxanes, vinca alkaloid, alkylating agent, platinum compound, proteasome inhibitor, nitrogen mustard, oestrogen analogue, monoclonal antibody, tyrosine kinase inhibitor, mTOR inhibitor, retinoid, immunomodulatory agent, histone deacetylase inhibitor, or a combination thereof.

43-45. (canceled)

46. The thermosensitive hydrogel according to claim 35, wherein the at least one therapeutic or diagnostic agent is not covalently conjugated to the thermosensitive polymer or polypeptide.

47. (canceled)

48. The thermosensitive hydrogel according to claim 35, wherein the thermosensitive polymer comprises a third portion that is covalently conjugated to the at least one therapeutic or diagnostic agent.

49. (canceled)

50. The thermosensitive hydrogel according to claim 48, wherein the third portion of the thermosensitive polymer comprises a linear polymer having one terminus covalently conjugated to the polypeptide, and a second terminus covalently conjugated to the therapeutic or diagnostic agent.

51. The thermosensitive hydrogel according to claim 48, wherein the third portion of the thermosensitive polymer comprises a branched polymer having two termini covalently conjugated to the polypeptide, and at least one terminus covalently conjugated to the therapeutic or diagnostic agent.

52-105. (canceled)

106. A method of making the thermosensitive hydrogel according to claim 1, comprising covalently conjugating the thermosensitive polymer to the polypeptide.

107. The method according to claim 106, comprising the step:
a) providing an activated thermosensitive polymer, and then reacting the activated thermosensitive polymer with the polypeptide;
b) providing an activated polypeptide, and then reacting the activated polypeptide with the thermosensitive polymer;
c) providing an activated thermosensitive polymer, and providing an activated polypeptide, and then reacting the activated thermosensitive polymer with the activated polypeptide; or
d) combining the thermosensitive polymer and polypeptide to give a mixture, and then adding an activating agent to the mixture.

108. The method according to claim 107, wherein the activated polymer has the structure:
Y¹—[CH₂]_{z}—[TP]—([CH₂]_{z}—Y²)_{y};
wherein
y is 1, 2, 3, 4, 5, 6 or 7,
TP represents a linear thermosensitive polymer when y is 1, and a branched thermosensitive polymer when y is 2, 3, 4, 5, 6, or 7;
Y¹ is
a) a therapeutic or diagnostic agent
b) a group having the formula: 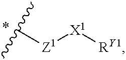
wherein X¹-R^{Y1} taken together represent OH, SH, NH₂, or —CH═CH₂; or
X¹ is C═O or SO₂, wherein R^{Y1} is selected from F, Cl, Br, I, OC_{1-8alkyl}, OC_{1-8aryl}, or a heterocycle having at least nitrogen atom, said nitrogen atom bonded to X¹, wherein each OC₁₋₈alkyl and OC₁₋₈aryl groups may be substituted one or more times by F, Cl, Br, I, NO₂
c) a maleimide having the formula: 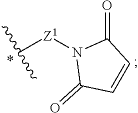
d) an 1,3-dipole or tetrazine; or
e) a cyclooctyne or trans-cyclooctene, optionally having the formula: 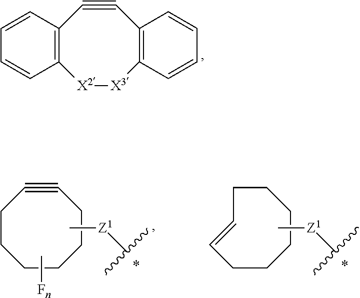
wherein one of X^{2′} and X^{3′} is N—Z¹—* or —CH—Z¹—*; and the other of X^{2′} and X^{3′} is CH₂ or C═O; or X² and X³ together represent a group having the formula: 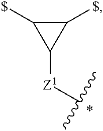
wherein each $ represents a point of attachment to the remainder of the cyclooctyne;
Y² is
a) a group having the formula: 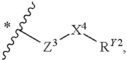
wherein X⁴-R^{Y2} taken together represent OH, SH, NH₂, or —CH═CH₂; or
R^{Y2} is selected from F, Cl, Br, I, OC₁₋₈alkyl, OC₁₋₈aryl, or a heterocycle having at least nitrogen atom, said nitrogen atom bonded to X⁴, wherein each OC₁₋₈alkyl and OC₁₋₈aryl groups may be substituted one or more times by F, Cl, Br, I, NO₂
b) a maleimide having the formula: 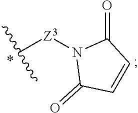
c) an 1,3-dipole or tetrazine; or
d) a cyclooctyne or trans-cyclooctene, optionally having the formula: 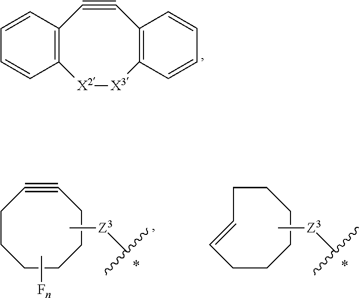
wherein one of X^{2′} and X^{3′} is N—Z³—* or —CH—Z³—*; and the other of X^{2′} and X^{3′} is CH₂ or C═O; or X² and X³ together represent a group having the formula: 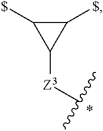
wherein each $ represents a point of attachment to the remainder of the cyclooctyne;
wherein * represents the point of attachment to the polymer; and
provided that both Y¹ and Y² are not H, and both Y¹ and Y² are not a therapeutic or diagnostic agent.

109. The method according to claim 107, wherein the activated polypeptide has the structure:
Y³—X^{aa}—[PP],
wherein
PP represents the polypeptide;
X^{aa} is selected from N, NH, S, O, C(O);
Y³ is selected from:
a) H;
b) OH;
c) a group having the formula: 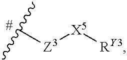
X³ is C═O or SO₂, wherein R^{Y3} is selected from F, Cl, Br, I, OC₁₋₈alkyl, OC₁₋₈aryl, or a heterocycle having at least nitrogen atom, said nitrogen atom bonded to X¹, wherein each OC₁₋₈alkyl and OC₁₋₈aryl groups may be substituted one or more times by F, Cl, Br, I, NO₂
d) a maleimide having the formula: 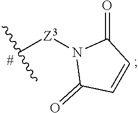
e) an 1,3-dipole or tetrazine; or
f) a cyclooctyne or trans-cyclooctene, optionally having the formula: 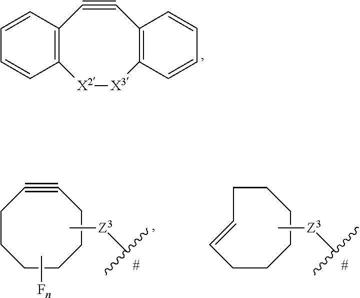
wherein one of X^{2′} and X^{3′} is N—Z³—^{#} or —CH—Z^{#—}; and the other of X^{2′} and X^{3′} is CH₂ or C═O; or X² and X³ together represent a group having the formula: 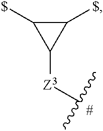
wherein each $ represents a point of attachment to the remainder of the cyclooctyne; and wherein # represents the point of attachment to X^{aa}.

110-112. (canceled)

113. A method of delivering a therapeutic or diagnostic agent to a subject in need thereof, comprising administering to the subject the thermosensitive hydrogel according to claim 35.

114-116. (canceled)

117. The method according to claim 113, wherein the thermosensitive hydrogel is administered as a sol, and undergoes phase transition to gel subsequent to administration.

118. (canceled)

119. A method of treating cancer in a patient in need thereof,
comprising administering to the patient the thermosensitive hydrogel according to claim 40.

120-131. (canceled)

132. A method of making a pharmaceutical dosage form, comprising rehydrate the thermosensitive hydrogel according to claim 1, wherein the thermosensitive hydrogel is in lyophilized form, in an aqueous solution, wherein the aqueous solution comprises at least one therapeutic or diagnostic agent.

133. (canceled)
